(19) United States
(12) Patent Application Publication
(10) Pub. No.: US 2023/0103731 A1
(43) Pub. Date: Apr. 6, 2023

(54) GENE VECTOR CONTROL BY CARDIOMYOCYTE-EXPRESSED MICRORNAS

(71) Applicant: Tenaya Therapeutics, Inc., South san Francisco, CA (US)

(72) Inventors: Laura LOMBARDI, South San Francisco, CA (US); Elena C. LEON, South San Francisco, CA (US); Tawny Neal QURESHI, South San Francisco, CA (US); Kathryn N. IVEY, South San Francisco, CA (US)

(21) Appl. No.: 17/908,458

(22) PCT Filed: Feb. 26, 2021
(86) PCT No.: PCT/US2021/020032

§ 371 Date: Aug. 31, 2022

Related U.S. Application Data
(60) Provisional application No. 62/984,183, filed on Mar. 2, 2020.

(51) Int. Cl. C12N 15/86; A61P 9/04; C07K 14/47

(52) U.S. Cl. CPC C12N 15/86; A61P 9/04; C07K 14/4705; C12N 2310/141; C12N 2750/14143; C12N 2750/14171; A61K 38/00

(57) ABSTRACT

The present disclosure provides vectors and method of use thereof, for cell-type specific repression of expression of transgenes (e.g., cardiomyocyte reprogramming factors) using microRNA binding sites.

### CROSS REFERENCE TO RELATED APPLICATIONS

This application claims the benefit of priority to U.S. Provisional Patent Application Ser. No. 62/984,183 filed on Mar. 2, 2020, the contents of which are hereby incorporated by reference in their entireties.

### REFERENCE TO SEQUENCE LISTING

This application is being filed electronically via EFS-Web and includes an electronically submitted sequence listing in .txt format. The .txt file contains a sequence listing entitled “TENA_017_01WO_SeqList_ST25.txt” created on Feb. 25, 2021 and having a size of 230 kilobytes. The sequence listing contained in this .txt file is part of the specification and is incorporated herein by reference in its entirety.

### FIELD OF THE INVENTION

The present disclosure relates generally to gene therapy vectors.

### BACKGROUND

Direct cardiac reprogramming has emerged as a strategy to create new cardiomyocytes, leading to improved heart function in patients diagnosed with or at risk for cardiomyopathy, heart failure, or other cardiac pathologies. Various combinations of genetic and chemical reprogramming factors have been shown to prompt reprogramming of other cells (such as fibroblasts) into cardiac cells (particularly cardiomyocytes). For example, a combination of three cardiac developmental transcription factors—GATA4, MEF2C, and TBXS (GMT)—can be used to reprogram dermal or cardiac fibroblasts to induced cardiomyocyte (iCM)-like cells in mice. GATA4, MEF2C, TBXS, MESP1, and MYOCD (GMTMM) when expressed together as a cocktail of factors change cell morphology from a spindle-like shape to a rod-like shape and causes cells to exhibit spontaneous Ca²⁺ oscillation. HAND2, NKX2.5, the microRNAs miR-1 and miR-133, JAK or TGF-β have been shown to enhance such reprogramming. In humans, supplementation of GMT with ETS2 and MESP1 induces cardiac-specific gene expression and sarcomere formation. Other combinations of factors for direct reprogramming have been described in the art, as reviewed in Srivastava and DeWitt. Cell 166:1386-96 (2016).

There remains, however, a need in the art for alternative and improved reprogramming methods as well as means for implementing those methods, such as vectors or vector systems. The present disclosure that addresses this unmet need.

### SUMMARY OF THE DISCLOSURE

The invention relates generally to vectors and method of use thereof, for cell-type specific repression of expression of transgenes (e.g., cardiomyocyte reprogramming factors). The disclosure provides vectors comprising microRNA binding site(s) configured to promote specific repression of expression of transgene (e.g., cardiomyocyte reprogramming factors) in cardiomyocyte and cardiomyocyte progenitor cells, compared to in cardiac fibroblasts. In some embodiments, the microRNA is selected for specific expression in induced cardiomyocytes by treating cardiac fibroblasts with an effective amount of a composition that induces reprogramming of cardiac fibroblasts to cardiomyocytes, and measuring the expression of one or more microRNAs in the cardiac fibroblasts to identified microRNAs that are expressed during the programming process and optionally late in the reprogramming process. In some embodiments, the selected microRNA is expressed in the cardiac fibroblasts only after a predetermined time. In some embodiments, tissue- and cell-type specific repression of expression permits expression in a target cell type (e.g., cardiac fibroblast) while repressing expression in a non-target cell type (e.g., cardiomyocyte). In some embodiments, the microRNA binding site(s) permit expression in the target cell for a sufficient time to cause effective conversion of the target cell into the non-target cell.

### BRIEF DESCRIPTION OF THE DRAWINGS

FIG. 1 shows a vector map according to an embodiment.

FIG. 2A shows repression of GFP transgene expression assessed by flow cytometry analysis four days after iPSC-CM transduction with AAV at MOI 160k.

FIG. 2B shows lack of repression of GFP transgene expression assessed by flow cytometry analysis four days after hCF transduction with AAV at MOI 160k.

FIG. 3 shows repression of GFP transgene expression assessed by flow cytometry analysis four days after iPSC-CMs transduction with AAV at MOI 500k.

FIG. 4 shows levels of microRNA expression determined by qPCR after treatment with My^{Δ3} A reprogramming cocktail, compared to the levels in human iPSC-CMs.

FIG. 5A shows flow cytometry analysis of iPSC-CMs four days after transduction with AAV (MOI 500k).

FIG. 5B shows flow cytometry analysis of hCFs two days after transduction with AAV (MOI 160k).

FIG. 5C shows immunofluorescence analysis after three weeks of reprogramming with AAV at MOI 640k.

FIG. 6A shows My^{Δ3} A with the miR-208 targeted cassette is efficacious against myocardial infarction by kinetic echocardiography data of % ejection fraction throughout the course of the study.

FIG. 6B shows echocardiography data from the close of the in vivo study at eight weeks post-injection indicating a significant increase in all groups relative to the GFP-encoding negative control (n=10-13 mice/group).

FIG. 6C shows an analysis of fibrosis based on trichrome staining in which muscle stains red and fibrotic tissue stains blue. Quantification of the % of the cardiac cross-section that is fibrotic across all groups indicated a significant reduction in all treatment groups (n=6-8 hearts/group, five sections/heart).

FIG. 6D shows representative trichrome-stained cardiac cross-sections from the negative control and AAV5z: My^{Δ3} A_208_4.

FIG. 7 shows a graph of the change in percent ejection fraction of treated and untreated pigs with ischemic injury, up to nine weeks post-injection

FIG. 8 shows flow cytometry analysis of iPSC-CMs four days after transduction with AAV (MOI 500k)

FIG. 9 shows a graph of percent ejection fraction in a rat study of ischemic injury, four weeks post-injection

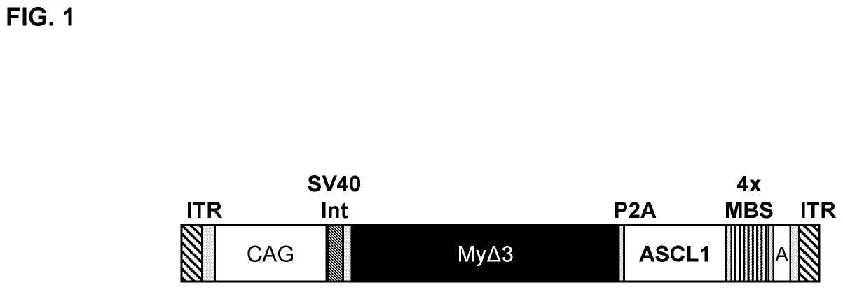

### DETAILED DESCRIPTION

The present inventors have recognized that inclusion of selected microRNA binding sites in gene-therapy vectors can be used to increase cell-type specificity of the vectors. The vectors of the disclosure employ binding sites for microRNAs that are expressed in cardiomyocytes or cardiomyocyte progenitors to repress expression of transgenes in those cell types. These vectors may increase efficacy and/or safety of in vivo gene therapy. In some embodiments, expression is repressed in other cell types (e.g., skeletal muscle) while being permitted and/or maintained in a target cell type. In some embodiments, the target cell type is a cardiac fibroblast. In some embodiments, the target cell type is a cell capable of being reprogramed into a cardiomyocyte (e.g., for in vivo cellular reprogramming). In some embodiments, the target cell type is a cell having a functional defect due to a loss-of-function mutation in a gene (e.g., for gene replacement therapy). Some embodiments of the vectors of the disclosure employ microRNA binding sites for microRNAs that are expressed late in the reprogramming process. In such embodiments, selection of the microRNA binding site permits expression of cardiomyocyte reprogramming factors from the vector without premature repression of expression.

The present disclosure further provides compositions and methods for generating cardiomyocytes from non-cardiomyocyte cells, for example, by direct reprogramming of cells into cardiomyocytes. The ability of selected microRNAs with limited or no capacity to reprogram cells into cardiomyocytes is increased when the selected microRNA is expressed with MYOCD and ASCL1, or with MYOCD alone. Additionally, the ability of MYOCD, ASCL1, or MYOCD and ASCL1 to reprogram cells into cardiomyocytes is increased by expression of a selected microRNA. Thus, the disclosure provides compositions capable of expressing MYOCD and a microRNA, or of expressing MYOCD, ASCL1, and a microRNA, and methods of use thereof. Advantageously, reprogramming differentiated cells (e.g., fibroblasts) into cardiomyocytes is enhanced compared to expression of these factors alone.

The disclosure provides compositions, such as vectors, comprising one or more polynucleotides collectively encoding a microRNA, a MYOCD protein, and optionally an ASCL1 protein. When a single vector is used, the coding polynucleotides can be provided in the vector in any 5′ to 3′ order and on the same or different polynucleotide strands within the vector. The disclosure further provides vector systems made up of more than one vector. Some vectors are polycistronic vectors—for example, 2A-linked polycistronic vectors, such as, without limitation, vectors comprising MYOCD-2A-ASCL1 or ASCL1-2A-MYPOCD polynucleotides.

The vectors include viral and non-viral vectors, such as, without limitation, a lipid nanoparticle, a transposon, an adeno-associated virus (AAV) vector, an adenovirus, a retrovirus, an integrating lentiviral vector (LVV), and a non-integrating LVV. Each of the polynucleotides optionally share sequence identity to a native, human polynucleotide sequence for the corresponding gene, or have a heterologous sequence encoding a protein identical to or sharing sequence identity to the corresponding native, human protein. In some embodiments, the MYOCD protein encoded by the MYOCD polynucleotide is an engineered myocardin. For example, MYOCD may be engineered to include an internal deletion that reduces its size but preserves its function.

The disclosure further provides methods of using the foregoing vectors and vector systems. Methods of use include methods of inducing a cardiomyocyte phenotype in differentiated cells (in vivo or in vitro) and methods of treating a heart condition in a subject suffering from, or at risk for, a heart condition. The disclosure further provides kits comprising vectors and vector systems with instructions for use in treating a heart condition.

The present disclosure provides methods and compositions relating to the generation of iCM cells (in vivo, in vitro, or ex vivo) by reprogramming other cell types. In particular, the present inventors have discovered that differentiated cells, for example, fibroblasts, can be reprogrammed into cardiomyocytes by expression of a microRNA and MYOCD and/or ASCL1.

A microRNA (“miRNA”) is a small non-coding RNA molecule that functions in RNA silencing and post-transcriptional regulation of gene expression via base pairing with complementary sequences within messenger RNA (mRNA) molecules. Under the standard nomenclature system, the prefix “miR” is followed by a dash and a number. “miR-” refers to the mature form of the miRNA; “mir-” refers to the primary mRNA (pri-miRNA) or the precursor miRNA (pre-miRNA); and “MIR” refers to the gene that encodes them. miRNAs with nearly identical sequences are annotated with an additional lower case letter. Species of origin is designated with a three-letter prefix, e.g., “hsa-” for human. miRNA genes that lead to identical mature miRNAs, but are located at different places in the genome, are indicated with an additional dash-number suffix, e.g. miR-194-1 and mir-194-2. Native human miRNAs are typically transcribed as the >100 nucleotide pri-miRNA, which is processed to form the pre-miRNA, which is further processed to form the mature miRNA.

miRNAs can be expressed from a vector, for example a viral vector, by operatively linking a sequence encoding the pre-miRNA to a promoter active in the host cell. For example, Cell Biolabs' pMXs retroviral expression vector is designed to clone and express an individual pri-miRNA while preserving putative hairpin structures to ensure biologically relevant interactions with endogenous processing machinery and regulatory partners, leading to properly cleaved microRNAs. The pri-miRNA may comprise the pre-miRNA plus about 150 bp of its own flanking sequence in each 5′ or 3′ side, or different flanking sequences can be used to produce the same mature miRNA according to methods known in the art. Exemplary microRNAs of interest include miR-133a-2, miR-133a-1, miR-19b-2, miR-19b-1, miR-326, miR-1-1, miR-1298, miR-133b, miR-1-2, miR-92a-2, miR-20b, miR-20a, miR-141, miR-155, miR-17, hsa-let-7c, miR-202, miR-200a, miR-206, miR-509-1, miR-509-2, miR-124-3, miR-124-2, miR-378a, miR-378e, miR-378h, miR-378i, miR-137, miR-671, miR-24-1, miR-182, miR-302d, miR-96, miR-30c-2, and miR-146b. The pri-miRNA sequences used to express the mature miRNAs are provided in Table 1, with the mature miRNA sequences in capitals.

TABLE 1

| | | | SEQ ID NO |
|---|---|---|---|
| | | SEQ ID NO | mature |
| microRNA | Sequence | pri-miRNA | miRNA |
| miR-133a-1 | acaatgctttgctagagctggtaaaatggaa | 65 | 100 |
| | ccaaatcgcctcttcaatggaTTTGGTCCCC | | |
| | TTCAACCAGCTGtagctatgcattga | | |
| miR-133a-2 | gggagccaaatgctttgctagagctggtaaa | 66 | 101 |
| | atggaaccaaatcgactgtccaatggaTTTG | | |
| | GTCCCCTTCAACCAGCTGtagctgtgcattg | | |
| | atggcgccg | | |
| miR-133b | cctcagaagaaagatgccccctgctctggct | 67 | 102 |
| | ggtcaaacggaaccaagtccgtcttcctgag | | |
| | aggTTTGGTCCCCTTCAACCAGCTAcagcag | | |
| | ggctggcaatgcccagtccttggaga | | |
| miR-19b-1 | cactgttctatggttagttttgcaggtttgc | 68 | 103 |
| | atccagctgtgtgatattctgcTGTGCAAAT | | |
| | CCATGCAAAACTGActgtggtagtg | | |
| miR-19b-2 | acattgctacttacaattagttttgcaggtt | 69 | 104 |
| | tgcatttcagcgtatatatgtatatgtggcT | | |
| | GTGCAAATCCATGCAAAACTGAttgtgataa | | |
| | tgt | | |
| miR-326 | ctcatctgtctgttgggctggaggcagggcc | 70 | 105 |
| | tttgtgaaggcgggtggtgctcagatcgCCT | | |
| | CTGGGCCCTTCCTCCAGccccgaggcggatt | | |
| | ca | | |
| miR-1-1 | tgggaaacatacttctttatatgcccatatg | 71 | 106 |
| | gacctgctaagctaTGGAATGTAAAGAAGTA | | |
| | TGTATctca | | |
| miR-1-2 | acctactcagagtacatacttctttatgtac | 72 | 107 |
| | ccatatgaacatacaatgctaTGGAATGTAA | | |
| | AGAAGTATGTATttttggtaggc | | |
| miR-1298 | agacgaggagttaagagTTCATTCGGCTGTC | 73 | 108 |
| | CAGATGTAtccaagtaccctgtgttatttgg | | |
| | caataaatacatctgggcaactgactgaact | | |
| | tttcacttttcatgactca | | |
| miR-92a-2 | tcatccctgggtggggatttgttgcattact | 74 | 109 |
| | tgtgttctatataaagTATTGCACTTGTCCC | | |
| | GGCCTGTggaaga | | |
| miR-20a | gtagcacTAAAGTGCTTATAGTGCAGGTAGt | 75 | 110 |
| | gtttagttatctactgcattatgagcactta | | |
| | aagtactgc | | |
| miR-20b | agtacCAAAGTGCTCATAGTGCAGGTAGttt | 76 | ill |
| | tggcatgactctactgtagtatgggcacttc | | |
| | cagtact | | |
| miR-141 | cggccggccctgggtccatcttccagtacag | 77 | 112 |
| | tgttggatggtctaattgtgaagctccTAAC | | |
| | ACTGTCTGGTAAAGATGGctcccgggtgggt | | |
| | tc | | |
| miR-155 | ctgTTAATGCTAATCGTGATAGGGGTTtttg | 78 | 113 |
| | cctccaactgactcctacatattagcattaa | | |
| | cag | | |
| miR-17 | gtcagaataatgtCAAAGTGCTTACAGTGCA | 79 | 114 |
| | GGTAGtgatatgtgcatctactgcagtgaag | | |
| | gcacttgtagcattatggtgac | | |
| hsa-let-7c | gcatccgggtTGAGGTAGTAGGTTGTATGGT | 80 | 115 |
| | Ttagagttacaccctgggagttaactgtaca | | |
| | accttctagctttccttggagc | | |
| miR-202 | cgcctcagagccgcccgccgttcctttTTCC | 81 | 116 |
| | TATGCATATACTTCTTTGaggatctggccta | | |
| | aagaggtatagggcatgggaaaacggggcgg | | |
| | tcgggtcctccccagcg | | |
| miR-200a | ccgggcccctgtgagcatcttaccggacagt | 82 | 117 |
| | gctggatttcccagcttgactcTAACACTGT | | |
| | CTGGTAACGATGTtcaaaggtgacccgc | | |
| miR-206 | tgcttcccgaggccacatgcttctttatatc | 83 | 118 |
| | cccatatggattactttgctaTGGAATGTAA | | |
| | GGAAGTGTGTGGtttcggcaagtg | | |
| miR-509-1 | catgctgtgtgtggtaccctactgcagacag | 84 | 119 |
| | tggcaatcatgtataattaaaaaTGATTGGT | | |
| | ACGTCTGTGGGTAGagtactgcatgacacat | | |
| | g | | |
| miR-509-2 | catgctgtgtgtggtaccctactgcagacag | 85 | 120 |
| | tggcaatcatgtataattaaaaaTGATTGGT | | |
| | ACGTCTGTGGGTAGagtactgcatgacac | | |
| miR-124-2 | atcaagattagaggctctgctctccgtgttc | 86 | 121 |
| | acagcggaccttgatttaatgtcatacaatT | | |
| | AAGGCACGCGGTGAATGCCAAgagcggagcc | | |
| | tacggctgcacttgaa | | |
| miR-124-3 | tgagggcccctctgcgtgttcacagcggacc | 87 | 122 |
| | ttgatttaatgtctatacaatTAAGGCACGC | | |
| | GGTGAATGCCAAgagaggcgcctcc | | |
| miR-378a | agggctcctgactccaggtcctgtgtgttac | 88 | 123 |
| | ctagaaatagcACTGGACTTGGAGTCAGAAG | | |
| | GCct | | |
| miR-378e | ctgactccagtgtccaggccaggggcagaca | 89 | 124 |
| | gtggacagagaacagtgcccaagaccACTGG | | |
| | ACTTGGAGTCAGGAcat | | |
| miR-378h | acaggaacACTGGACTTGGTGTCAGATGGga | 90 | 125 |
| | tgagccctggctctgtttcctagcagcaatc | | |
| | tgatcttgagctagtcactgg | | |
| miR-378i | gggagcACTGGACTAGGAGTCAGAAGGtgga | 91 | 126 |
| | gttctgggtgctgttttcccactcttgggcc | | |
| | ctgggcatgttctg | | |
| miR-137 | ggtcctctgactctcttcggtgacgggtatt | 92 | 127 |
| | cttgggtggataatacggattacgttgTTAT | | |
| | TGCTTAAGAATACGCGTAGtcgaggagagta | | |
| | ccagcggca | | |
| miR-671 | gcaggtgaactggcaggccaggaagaggAGG | 93 | 128 |
| | AAGCCCTGGAGGGGCTGGAGgtgatggatgt | | |
| | tttcctccggttctcagggctccacctcttt | | |
| | cgggccgtagagccagggctggtgc | | |
| miR-24-1 | ctccggtgcctactgagctgatatcagttct | 94 | 129 |
| | cattttacacacTGGCTCAGTTCAGCAGGAA | | |
| | CAGgag | | |
| miR-182 | gagctgcttgcctccccccgttTTTGGCAAT | 95 | 130 |
| | GGTAGAACTCACACTggtgaggtaacaggat | | |
| | ccggtggttctagacttgccaactatggggc | | |
| | gaggactcagccggcac | | |
| miR-302d | cctctactttaacatggaggcacttgctgtg | 96 | 131 |
| | acatgacaaaaaTAAGTGCTTCCATGTTTGA | | |
| | GTGTgg | | |
| miR-96 | tggccgatTTTGGCACTAGCACATTTTTGCT | 97 | 132 |
| | tgtgtctctccgctctgagcaatcatgtgca | | |
| | gtgccaatatgggaaa | | |
| miR-30c-2 | agatacTGTAAACATCCTACACTCTCAGCtg | 98 | 133 |
| | tggaaagtaagaaagctgggagaaggctgtt | | |
| | tactctttct | | |
| miR-146b | cctggcacTGAGAACTGAATTCCATAGGCTG | 99 | 134 |
| | tgagctctagcaatgccctgtggactcagtt | | |
| | ctggtgcccgg | | |

Myocardin (MYOCD) is a smooth muscle and cardiac muscle-specific transcriptional coactivator of serum response factor. When expressed ectopically in nonmuscle cells, MYOCD can induce smooth muscle differentiation by its association with serum response factor. Du et al. MYOCD is a critical serum response factor cofactor in the transcriptional program regulating smooth muscle cell differentiation. Mol. Cell. Biol. 23:2425-37 (2003).

Achaete-scute family bHLH transcription factor 1 (ASCL1) is known primarily for its role in nervous system, neuronal, and neuroendocrine development. In the context of cellular reprogramming, ASCL1 is known in the art as a factor associated with conversion of nonneuronal cells into functional neurons. Indeed, expression of ASCL1 in conjunction with other reprogramming factors has been used in the art to convert human-induced pluripotent stem cells (hiPSCs) from a cardiomyocyte phenotype to a neuronal (Tuj1+cTnT−) or neuronal-like phenotype (Tuj1+cTnT+)—a contrary effect of reprogramming cardiomyocytes. In contrast, the present disclosure provides compositions and methods from generating iCM cells from fibroblasts using ASCL1.

### I. Definitions

As used herein, the term “functional cardiomyocyte” refers to a differentiated cardiomyocyte that is able to send or receive electrical signals. In some embodiments, a cardiomyocyte is said to be a functional cardiomyocyte if it exhibits electrophysiological properties such as action potentials and/or Ca²⁺ transients.

As used herein, a “differentiated non-cardiac cell” can refer to a cell that is not able to differentiate into all cell types of an adult organism (i.e., is not a pluripotent cell), and which is of a cellular lineage other than a cardiac lineage (e.g., a neuronal lineage or a connective tissue lineage). Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

As used herein, “protein-coding gene” means, when referring to a component of a vector, a polynucleotide that encodes a protein, other than a gene associated with the function of the vector. For example, the term protein-coding gene would encompass a polynucleotide encoding a human protein, or functional variant thereof, with reprogramming activity. It is intended that the phrase “the vector comprising no other protein-coding gene” in reference to a vector means that the vector comprises a polynucleotide(s) encoding the protein of interest(s) that is listed, but no polynucleotide encoding another protein that has reprogramming activity—such as other proteins known in the art to promote either a pluripotent or a cardiomyocyte phenotype. The phrase “the vector comprising no other protein-coding gene” does not exclude polynucleotides encoding proteins required for function of the vector, which optionally may be present, nor does the phrase exclude polynucleotides that do not encode proteins. Such vectors will include non-coding polynucleotide sequences and may include polynucleotides encoding RNA molecules (such as microRNAs). Conversely, when only certain protein-coding genes are listed, it is implied that other protein-coding genes may additionally be present, such as protein-coding genes that encode proteins that further promote reprogramming.

A “somatic cell” is a cell forming the body of an organism. Somatic cells include cells making up organs, skin, blood, bones and connective tissue in an organism, but not germ cells.

The terms “cardiac pathology” or “cardiac dysfunction” are used interchangeably and refer to any impairment in the heart's pumping function. This includes, for example, impairments in contractility, impairments in the ability to relax (sometimes referred to as diastolic dysfunction), abnormal or improper functioning of the heart's valves, diseases of the heart muscle (sometimes referred to as cardiomyopathies), diseases such as angina pectoris, myocardial ischemia and/or infarction characterized by inadequate blood supply to the heart muscle, infiltrative diseases such as amyloidosis and hemochromatosis, global or regional hypertrophy (such as may occur in some kinds of cardiomyopathy or systemic hypertension), and abnormal communications between chambers of the heart.

As used herein, the term “cardiomyopathy” refers to any disease or dysfunction of the myocardium (heart muscle) in which the heart is abnormally enlarged, thickened and/or stiffened. As a result, the heart muscle's ability to pump blood is usually weakened. The etiology of the disease or disorder may be, for example, inflammatory, metabolic, toxic, infiltrative, fibroplastic, hematological, genetic, or unknown in origin. There are two general types of cardiomyopathies: ischemic (resulting from a lack of oxygen) and non-ischemic.

As used herein, the term “gene of interest” refers to a reprogramming factor or to nucleic acid encoding the reprogramming factor. For example, when the reprogramming factor is a protein, the gene of interest is either—as apparent from context—a protein or the corresponding protein-coding polynucleotide sequence. Introduction, administration, or other use of gene of interest should be understood to refer to any means of increasing the expression of, or increasing the activity of, a gene, gene product, or functional variant of a gene product. Thus, in some embodiments, the disclosure provides methods of generating iCM cells comprising introducing a polynucleotide of interest, e.g. ASCL1 and/or MYOCD, as a nucleic acid (e.g. deoxyribonucleotide (DNA) or ribonucleotide (RNA)) into a target cell as a polynucleotide (e.g. deoxyribonucleotide (DNA) or ribonucleotide (RNA)). The polynucleotide may be introduced into a cell in any of the various means known in the art, including without limitation in a viral, non-viral vector, by contacting the cell with naked polynucleotide or polynucleotide in complex with a transfection reagent, or by electroporation. Use of a gene of interest as a nucleic acid may also include indirect alteration of the expression or activity of the gene of interest, such as gene-editing of the locus encoding the endogenous gene, expression of transcription or regulatory factors, contacting cells with a small-molecule activator of the gene of interest, or use of gene-editing methods, including DNA- or RNA-based methods, to alter the expression or activity of the gene of interest as a nucleic acid. In some embodiments, the methods of the disclosure include de-repressing transcription of a gene of interest by editing regulatory regions (e.g. enhancers or promoters), altering splice sites, removing or inserting microRNA recognition sites, administering an antagomir to repress a microRNA, administering a microRNA mimetic, or any other various means of modulating expression or activity of the gene of interest.

As used herein, “microRNA” refers to the mature microRNA. A polynucleotide encoding a microRNA generally refers to any polynucleotide whose expression in a host cell results in formation of the mature microRNA in that cell. A polynucleotide encoding a microRNA may share 100% sequence identity with the corresponding pre-RNA. One or more substitutions in the loop between the stems that encodes the mature microRNA sequences are, in some cases, tolerated. Although either strand of the duplex may potentially act as a functional miRNA, only one strand is usually incorporated into the RNA-induced silencing complex (RISC) where the miRNA and its mRNA target interact. Conventional techniques for using microRNAs are provided, for example, in Lawrie, ed. (2013) MicroRNAs in Medicine.

As used herein, the terms “subject” or “patient” refers to any animal, such as a domesticated animal, a zoo animal, or a human. The “subject” or “patient” can be a mammal such as a dog, cat, horse, livestock, a zoo animal, or a human. The subject or patient can also be any domesticated animal such as a bird, a pet, or a farm animal. Specific examples of “subjects” and “patients” include, but are not limited to, individuals with a cardiac disease or disorder, and individuals with cardiac disorder-related characteristics or symptoms.

The practice of the present disclosure will employ, unless otherwise indicated, conventional techniques of tissue culture, immunology, molecular biology, cell biology and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a cardiomyocyte” includes a plurality of cardiomyocytes.

Also as used herein, “and/or” refers to and encompasses any and all possible combinations of one or more of the associated listed items, as well as the lack of combinations when interpreted in the alternative (“or”).

“Administration,” “administering” and the like, when used in connection with a composition of the invention refer both to direct administration, which may be administration to non-cardiomyocytes in vitro, administration to non-cardiomyocytes in vivo, administration to a subject by a medical professional or by self-administration by the subject and/or to indirect administration, which may be the act of prescribing a composition of the invention. When used herein in reference to a cell, it refers to introducing a composition to the cell. Typically, an effective amount is administered, which amount can be determined by one of skill in the art. Any method of administration may be used. Small molecules may be administered to the cells by, for example, addition of the small molecules to the cell culture media or injection in vivo to the site of cardiac injury. Administration to a subject can be achieved by, for example, intravascular injection, intramyocardial delivery, and the like.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure. Cardiac cells may be derived from stem cells, including, for example, embryonic stem cells or induced pluripotent stem cells.

The term “cardiomyocyte” or “cardiomyocytes” as used herein refers to sarcomere-containing striated muscle cells, naturally found in the mammalian heart, as opposed to skeletal muscle cells. Cardiomyocytes are characterized by the expression of specialized molecules e.g., proteins like myosin heavy chain, myosin light chain, cardiac α-actinin. The term “cardiomyocyte” as used herein is an umbrella term comprising any cardiomyocyte subpopulation or cardiomyocyte subtype, e.g., atrial, ventricular and pacemaker cardiomyocytes.

The term “cardiomyocyte-like cells” is intended to mean cells sharing features with cardiomyocytes, but which may not share all features. For example, a cardiomyocyte-like cell may differ from a cardiomyocyte in expression of certain cardiac genes.

The term “culture” or “cell culture” means the maintenance of cells in an artificial, in vitro environment. A “cell culture system” is used herein to refer to culture conditions in which a population of cells may be grown as monolayers or in suspension. “Culture medium” is used herein to refer to a nutrient solution for the culturing, growth, or proliferation of cells. Culture medium may be characterized by functional properties such as, but not limited to, the ability to maintain cells in a particular state (e.g., a pluripotent state, a quiescent state, etc.), or to mature cells, such as, in some embodiments, to promote the differentiation of progenitor cells into cells of a particular lineage (e.g., a cardiomyocyte).

As used herein, the term “expression” or “express” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample.

As used herein, an “expression cassette” is a DNA polynucleotide comprising one or more polynucleotide encoding protein(s) or nucleic acid(s) that is configured to express the polynucleotide in a host cell. Typically, expression of the polynucleotide(s) is placed under the control of certain regulatory elements, including constitutive or inducible promoters, tissue-specific regulatory elements, and enhancers. Such polynucleotides are said to be “operably linked to” or “operatively linked to” the regulatory elements (e.g., a promoter).

The term “induced cardiomyocyte” or the abbreviation “iCM” refers to a non-cardiomyocyte (and its progeny) that has been transformed into a cardiomyocyte (and/or cardiomyocyte-like cell). The methods of the present disclosure can be used in conjunction with any methods now known or later discovered for generating induced cardiomyocytes, for example, to enhance other techniques.

The term “non-cardiomyocyte” as used herein refers to any cell or population of cells in a cell preparation not fulfilling the criteria of a “cardiomyocyte” as defined and used herein. Non-limiting examples of non-cardiomyocytes include somatic cells, cardiac fibroblasts, non-cardiac fibroblasts, cardiac progenitor cells, and stem cells.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “regenerate,” “regeneration” and the like as used herein in the context of injured cardiac tissue shall be given their ordinary meanings and shall also refer to the process of growing and/or developing new cardiac tissue in a heart or cardiac tissue that has been injured, for example, injured due to ischemia, infarction, reperfusion, or other disease. In some embodiments, cardiac tissue regeneration comprises generation of cardiomyocytes.

As used herein, the term “reprogramming” or “transdifferentiation” refers to the generation of a cell of a certain lineage (e.g., a cardiac cell) from a different type of cell (e.g., a fibroblast cell) without an intermediate process of de-differentiating the cell into a cell exhibiting pluripotent stem cell characteristics. As used herein “reprogramming” includes transdifferentiation, dedifferentiation and the like.

As used herein, “reprogramming activity” refers to the ability of a protein or polynucleotide having reprogramming activity to induce or to promote reprogramming of a cell into a cardiomyocyte or cardiomyocyte-like cell when it is introduced into or expressed by the cell, alone or in combination with other proteins or polynucleotides having reprogramming activity. For example, a first protein has reprogramming activity if expression of the first protein in a cell with no other factors induces or promotes reprogramming of the cell; but the first protein also has reprogramming activity, as the term is used herein, if the first protein promotes reprogramming in combination with a second protein—that is, when both the first protein and the second protein are expressed together.

As used herein, the term “reprogramming efficiency” refers to the number of cells in a sample that are successfully reprogrammed to cardiomyocytes relative to the total number of cells in the sample.

The term “reprogramming factor” as used herein includes a factor that is introduced for expression in a cell to assist in the reprogramming of the cell into an induced cardiomyocyte. Reprogramming factors include proteins and nucleic acids (e.g., RNAs such as microRNAs, siRNA, or shRNAs).

The term “stem cells” refer to cells that have the capacity to self-renew and to generate differentiated progeny. The term “pluripotent stem cells” refers to stem cells that can give rise to cells of all three germ layers (endoderm, mesoderm and ectoderm), but do not have the capacity to give rise to a complete organism.

“Treatment,” “treating,” and “treat” are defined as acting upon a disease, disorder, or condition with an agent to reduce or ameliorate harmful or any other undesired effects of the disease, disorder, condition and/or their symptoms.

As used herein, the term “effective amount” and the like refers to an amount that is sufficient to induce a desired physiologic outcome (e.g., reprogramming of a cell or treatment of a disease). An effective amount can be administered in one or more administrations, applications or dosages. Such delivery is dependent on a number of variables including the time period which the individual dosage unit is to be used, the bioavailability of the composition, the route of administration, etc. It is understood, however, that specific amounts of the compositions (e.g., reprogramming factors) for any particular subject depends upon a variety of factors including the activity of the specific agent employed, the age, body weight, general health, sex, and diet of the subject, the time of administration, the rate of excretion, the composition combination, severity of the particular disease being treated and form of administration.

As used herein, the term “equivalents thereof” in reference to a polypeptide or nucleic acid sequence refers to a polypeptide or nucleic acid that differs from a reference polypeptide or nucleic acid sequence, but retains essential properties (e.g., biological activity). A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, deletions, additions, fusions and truncations in the polypeptide encoded by the reference sequence. Generally, differences are limited so that the sequences of the reference polypeptide and the variant are closely similar overall and, in many regions, identical.

The term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, or cell does not require “isolation” to distinguish it from its naturally occurring counterpart.

As used herein, the term “nucleic acid” and “polynucleotide” are used interchangeably and refer to a polymeric form of nucleotides of any length, either deoxyribonucleotides or ribonucleotides, or analogs thereof. Non-limiting examples of polynucleotides include linear and circular nucleic acids, messenger RNA (mRNA), cDNA, recombinant polynucleotides, vectors, probes, and primers.

The terms “polypeptide,” “peptide,” and “protein,” are used interchangeably herein and refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues, immunologically tagged proteins, and the like.

As used herein, the word “polynucleotide” preceded by a gene name (for example, “MYOCD polynucleotide”) refers to a polynucleotide sequence encoding the corresponding protein (for example, a “MYOCD protein”).

As used herein, the word “protein” preceded by a gene name (for example, “MYOCD protein”) refers to either the native protein or a functional variant thereof. A “native protein” is a protein encoded by a genomic copy of a gene of an organism, preferably the organism for which the vector is intended (e.g., a human, a rodent, a primate, or an animal of veterinary interest), in any of the gene's functional isoforms or functional allelic variations.

As used herein, a “functional variant” of a protein is a variant with any number of amino acid substitutions that retains the functional attributes of the protein, including, e.g., the protein's ability to induce, in combination with other factors, the reprogramming of cells into cardiomyocytes. Functional variants can be identified computationally, such as variants having only conservative substitutions, or experimentally using in vitro or in vivo assays.

As used herein, the term “progenitor cell” refers to a cell that is committed to differentiate into a specific type of cell or to form a specific type of tissue. A progenitor cell, like a stem cell, can further differentiate into one or more kinds of cells, but is more mature than a stem cell such that it has a more limited/restricted differentiation capacity.

The term “vector” refers to a macromolecule or complex of molecules comprising a polynucleotide or protein to be delivered to a host cell, either in vitro or in vivo.

As used herein, the term “viral vector” refers either to a nucleic acid molecule that includes virus-derived nucleic acid elements that typically facilitate transfer of the nucleic acid molecule or integration into the genome of a cell or to a viral particle that mediates nucleic acid transfer. Viral particles will typically include various viral components and sometimes also cell components in addition to nucleic acid(s).

The term “genetic modification” refers to a permanent or transient genetic change induced in a cell following introduction of new nucleic acid (i.e., nucleic acid exogenous to the cell). Genetic change can be accomplished by incorporation of the new nucleic acid into the genome of the cardiac cell, or by transient or stable maintenance of the new nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, and the like.

The term “stem cells” refer to cells that have the capacity to self-renew and to generate differentiated progeny. The term “pluripotent stem cells” refers to stem cells that can give rise to cells of all three germ layers (endoderm, mesoderm and ectoderm), but do not have the capacity to give rise to a complete organism. In some embodiments, the compositions for inducing cardiomyocyte phenotype can be used on a population of cells to induce reprogramming. In other embodiments, the compositions induce a cardiomyocyte phenotype.

The term “induced pluripotent stem cells” shall be given its ordinary meaning and shall also refer to differentiated mammalian somatic cells (e.g., adult somatic cells, such as skin) that have been reprogrammed to exhibit at least one characteristic of pluripotency. See, for example, Takahashi et al. (2007) Cell 131(5):861-872, Kim et al. (2011) Proc. Natl. Acad. Sci. 108(19): 7838-7843, Sell (2013) Stem Cells Handbook.

Unless stated otherwise, the abbreviations used throughout the specification have the following meanings: AHCF, adult human cardiac fibroblast, APCF, adult pig cardiac fibroblast, a-MHC-GFP, alpha-myosin heavy chain green fluorescence protein; CF, cardiac fibroblast; cm, centimeter; CO, cardiac output; EF, ejection fraction; FACS, fluorescence activated cell sorting; GFP, green fluorescence protein; GMT, Gata4, Mef2c and Tbx5; GMTc, Gata4, Mef2c, Tbx5, TGF-βi, WNTi; GO, gene ontology; HCF, human cardiac fibroblast; iCM, induced cardiomyocyte; kg, kilogram; μg, microgram; μl, microliter; mg, milligram; ml, milliliter; MI, myocardial infarction; msec, millisecond; min, minute; MyAMT, Myocardin, Ascl1, Mef2c and Tbx5; MyA, Myocardin and Ascl1; MyMT, Myocardin, Mef2c and Tbx5; MyMTc, Myocardin, Mef2c, Tbx5, TGF-βi, WNTi; MRI, magnetic resonance imaging; PBS, phosphate buffered saline; PBST, phosphate buffered saline, triton; PFA, paraformaldehyde; qPCR, quantitative polymerase chain reaction; qRT-PCR, quantitative reverse transcriptase polymerase chain reaction; RNA, ribonucleic acid; RNA-seq, RNA sequencing; RT-PCR, reverse transcriptase polymerase chain reaction; sec, second; SV, stroke volume; TGF-β, transforming growth factor beta; TGF-βi, transforming growth factor beta inhibitor; WNT, wingless-Int; WNTi, wingless-Int inhibitor; YFP, yellow fluorescence protein; 4F, Gata4, Mef2c, TBX5, and Myocardin; 4Fc, Gata4, Mef2c, TBX5, and Myocardin+TGF-βi and WNTi; 7F, Gata4, Mef2c, and Tbx5, Esrrg, Myocardin, Zfpm2, and Mesp1; 7Fc, Gata4, Mef2c, and Tbx5, Esrrg, Myocardin, Zfpm2, and Mesp1+TGF-βi and WNTi.

The detailed description of the disclosure is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated by reference to disclose and describe the methods and/or materials in connection with which the publications are cited.

### II. Transgenes and MicroRNA Binding Sites

The vectors of the disclosure may be designed for delivery of one or more transgenes to cells, including without limitation BMPR2, COL1A1, COL1A2, COL3A1, ELN, FGF1, FGF4, GGF2, HGF, OPRL1, PCSK9, RXFP1, SDF1, TGFBR2, TIMP3, TIMP4, VEGFA and/or others. That is, the vector may comprise a polynucleotide sequence encoding one or more of BMPR2, COL1A1, COL1A2, COL3A1, ELN, FGF1, FGF4, GGF2, HGF, OPRL1, PCSK9, RXFP1, SDF1, TGFBR2, TIMP3, TIMP4, VEGFA and/or others. The vectors of the disclosure are not limited to any particular set of transgenes. Transgenes may be protein-coding, DNA-coding, or RNA-coding. The one or more transgenes may include a gene-editing system, e.g. a CRISPR-Cas system.

### Reprogramming Factors

In some embodiments, the one or more transgenes comprises one or more reprogramming factors (e.g. cardiomyocyte reprogramming factors). The vectors may be used to delivery all of the factors needed achieve a desired cellular reprogramming elected, or selected members of such a set of reprogramming factors. For example, multiple vectors may be co-administered, only one of which comprises the selected microRNA binding site. Where the expression of multiple factors is needed to collectively generate a desired effect, the same or similar efficacy and/or safety benefits may be seen when only certain members of the set of factors is controlled by the microRNA binding site(s), or when all are controlled by the microRNA binding site(s).

In some embodiments, the present disclosure provides reprogramming factors and compositions thereof that are capable of modulating the expression of one or more genes such as polynucleotides or proteins of interest. The present inventors have surprisingly discovered that differentiated cells can be reprogrammed into iCM cells using one or more reprogramming factors that modulate the expression of one or more genes such as polynucleotides or proteins of interest, such as ASCL1, and/or MYOCD; and optionally a polynucleotide encoding a microRNA, where one or more of the foregoing polynucleotides comprises a selected by a microRNA binding site. In some embodiments, the one or more reprogramming factors are provided as a polynucleotide (e.g., an RNA, an mRNA, or a DNA polynucleotide) that encodes one or more transgenes. In some embodiments, one or more reprogramming factors are provided as a protein. In some embodiments, the polynucleotide encoding a microRNA shares perfect identity to the corresponding pre-microRNA.

In some embodiments, the one or more reprogramming factors provided herein modulate (e.g., increase or decrease) the expression of one or more proteins of interest. In some embodiments, the one or more target proteins are known to be involved in cardiomyocyte differentiation, proliferation, and/or function. In some embodiments, the one or more target polynucleotides or proteins are MYOCD/MYOCD and/or ASCL1/ASCL1. Illustrative gene sequences useful in the compositions and methods of the present disclosure are provided in Table 2. Where more than one isoform of a given gene of interest is known, it will be understood that embodiments of the present disclosure include compositions and methods that comprise the alternative isoforms of each gene of interest. The compositions and methods of the disclosure are not limited to the disclosed sequences, which are provided for example and illustration and are non-limiting.

In some embodiments, the present disclosure provides a reprogramming factor that modulates the expression of one or more genes of interest selected from ASCL1, MYOCD, MEF2C, and TBX5. In some embodiments, the reprogramming factors disclosed herein modulate the expression of one or more genes of interest selected from ASCL1, MYOCD, MEF2C, AND TBX5, CCNB1, CCND1, CDK1, CDK4, AURKB, OCT4, BAF60C, ESRRG, GATA4, GATA6, HAND2, IRX4, ISLL, MESP1, MESP2, NKX2.5, SRF, TBX20, ZFPM2, and MIR-133.

In some embodiments, the reprogramming factors disclosed herein modulate the expression of one or more genes of interest selected from GATA4, MEF2C, and TBX5 (i.e., GMT). In some embodiments, the reprogramming factors disclosed herein modulate the expression of one or more genes of interest selected from MYOCD, MEF2C, and TBX5 (i.e., MyMT). In some embodiments, the reprogramming factors disclosed herein modulate the expression of one or more genes of interest selected from MYOCD, ASCL1, MEF2C, and TBX5 (i.e., MyAMT). In some embodiments, the reprogramming factors disclosed herein modulate the expression of one or more genes of interest selected from MYOCD and ASCL1 (i.e., MyA). In some embodiments, the reprogramming factors disclosed herein modulate the expression of one or more genes of interest selected from GATA4, MEF2C, TBX5, and MYOCD (i.e., 4F). In other embodiments, the reprogramming factors disclosed herein modulate the expression of one or more genes of interest selected from GATA4, MEF2C, TBX5, ESRRG, MYOCD, ZFPM2, and MESP1 (i.e., 7F).

In some embodiments, the present disclosure provides a reprogramming factor that modulates the expression of one or more genes of interest selected from ASCL1, MYOCD, MEF2C, TBX5, DLX3, DLX6, GATA2, and GATA5.

TABLE 2
Representative Sequences

| | | Nucleotide (Open |
|---|---|---|
| | Protein | Reading Frame) |
| ASCL1 | SEQ ID NO: 1 | SEQ ID NO: 2 |
| DLX3 | SEQ ID NO: 43 | SEQ ID NO: 44 |
| DLX6 | SEQ ID NO: 45 | SEQ ID NO: 46 |
| ESRRG | SEQ ID NO: 47 | SEQ ID NO: 48 |
| GATA2 | SEQ ID NO: 49 | SEQ ID NO: 50 |
| GATA4 | SEQ ID NO: 51 | SEQ ID NO: 52 |
| MESP1 | SEQ ID NO: 53 | SEQ ID NO: 54 |
| MYF6 | SEQ ID NO: 55 | SEQ ID NO: 56 |
| MYOCD | SEQ ID NO: 3 | SEQ ID NO: 4 |
| MEF2C | SEQ ID NO: 5 | SEQ ID NO: 6 |
| TBX5 | SEQ ID NO: 7 | SEQ ID NO: 8 |

### Engineered Myocardins (MYOCDs)

In another aspect, the disclosure relates to engineered variants of MYOCD, such as an engineered MYOCD expressed from a smaller open reading frame, as described in U.S. Provisional Pat. Appl. No. 62/788,479. Applicant has found that MYOCD comprising an internal deletion retains the expression and function of MYOCD protein and MYOCD comprising an internal deletion can be used alone or in combination with other reprogramming factors (e.g., for generating cardiomyocytes from fibroblasts). In some embodiments of the present disclosure, the engineered MYOCD protein comprises a deletion of at least 50 amino acids in the region corresponding to amino acids 414-764 of the native MYOCD (SEQ ID NO: 3). In some embodiments, the engineered MYOCD is selected from one or three MYOCD variants with internal deletions: MyΔ1 having a deletion of residues 414 to 763 (SEQ ID NO: 14); MyΔ2 having a deletion of residues 439 to 763 (SEQ ID NO: 15); and preferably MyA3 having a deletion of residues 560 to 763 (SEQ ID NO: 16).

In some embodiments, the MYOCD polynucleotide is an engineered MYOCD polynucleotide. “MYOCD” or “myocardin” refers to either an engineered MYOCD protein or preferably a native MYOCD. In some embodiments, the engineered MYOCD polynucleotide encodes an engineered MYOCD protein having a length of at most 500, 550, 600, 650, 700, 750, 800, 850 or any number therebetween of amino acids. In some embodiments, the engineered MYOCD protein comprises an SRF interaction domain, an SAP domain, and a TAD domain. In some embodiments, the engineered MYOCD protein further comprises a Mef2C interaction domain. In some embodiments, the engineered MYOCD polynucleotide encodes an engineered MYOCD protein, with a deletion of at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or 350 amino acids in the region corresponding to amino acids 414-764 of the native myocardin (SEQ ID NO: 3). Various sequences used in the engineering of myocardin are provided in Table 3. In some embodiments, about four N-terminal residues of MYOCD are omitted or altered as inter-species conservation of MYOCD begins at residue 5. In some embodiments, further residues from the N terminus of MYOCD are omitted or altered.

TABLE 3
Sequences Used in Engineering of MYOCD

| Protein | SEQ ID NO. |
|---|---|
| Native MYOCD | SEQ ID NO: 3 |
| MYOCD 5-413 | SEQ ID NO: 10 |
| MYOCD 764-986 | SEQ ID NO: 11 |
| MYOCD 5-438 | SEQ ID NO: 12 |
| MYOCD 1-559 | SEQ ID NO: 13 |
| MYOCD 1-413, 764-986 (MyΔ1) | SEQ ID NO: 14 |
| MYOCD 1-438, 764-986 (MyΔ2) | SEQ ID NO: 15 |
| MYOCD 1-559, 764-986 (MyΔ3) | SEQ ID NO: 16 |
| Mef2c interaction domain (5-120) | SEQ ID NO: 17 |
| SRF domain (210-320) | SEQ ID NO: 18 |
| SAP domain (360-413) | SEQ ID NO: 19 |
| LZ domain (510-550) | SEQ ID NO: 20 |
| TAD domain (764-986) | SEQ ID NO: 11 |

In some embodiments, the engineered myocardin protein comprises one or more of an Mef2c interaction domain, an SRF domain, an SAP domain, an LZ domain, and a TAD domain. In some embodiments, the engineered myocardin protein comprises an Mef2c interaction domain, an SRF domain, an SAP domain, an LZ domain, and a TAD domain. In some embodiments, the engineered myocardin protein comprises an Mef2c interaction domain, an SRF domain, an SAP domain, and a TAD domain. In some embodiments, the engineered myocardin protein comprises an SRF domain, an SAP domain, an LZ domain, and a TAD domain. In some embodiments, the engineered myocardin protein comprises an SRF domain, an SAP domain, and a TAD domain.

In some embodiments, the engineered MYOCD is provided as a polynucleotide encoding the engineered MYOCD and, optionally, one or more other proteins of interest. In some embodiments, the polynucleotides are RNA, DNA, or mRNA polynucleotides. In some embodiments, the MYOCD polynucleotide shares identity with any of the isoforms of MYOCD In some embodiments, the MYOCD polynucleotide encodes an engineered MYOCD protein that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence selected from MyΔ1 (SEQ ID NO: 14), MyΔ2 (SEQ ID NO: 15), and MyΔ3 (SEQ ID NO: 16). In some embodiments, the engineered MYOCD protein comprises at least 2, 3, 4, 5, of a Mef2c interaction domain, a SRF domain, an SAP domain, an LZ domain, and a TAD domain. In some embodiments, the Mef2c interaction domain is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 17. In some embodiments, the SRF domain is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 18. In some embodiments, the SAP domain is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19. In some embodiments, the LZ domain is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20. In some embodiments, the TAD domain is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 11.

In some embodiments, the engineered MYOCD protein comprises two or more fragments of the native MYOCD linked by linkers. In general, a linker refers either to a peptide bond or a polypeptide sequence. In some embodiments other linkers are used, such as any of various chemical linkers used in peptide chemistry known in the art. Reference to a “peptide bond” means that two sequences are joined together to generate a composite sequence without any intervening amino acid residues. For example, MyΔ3 (SEQ ID NO: 16) comprises MYOCD 1-559 (SEQ ID NO: 13) joined by a peptide bond to MYOCD 764-986 (SEQ ID NO: 11). In some embodiments, the linker is any of various polypeptides used as linkers in the art; for example, without limitation, glycine-serine linkers such as G, GG, GGG, GSS, GGS, GGSGGS (SEQ ID NO: 30), GSSGGS (SEQ ID NO: 31), GGSGSS (SEQ ID NO: 32), GGSGGSGGS (SEQ ID NO: 33), GGSGGSGGSGGS (SEQ ID NO: 34). In some embodiments, the linker is a domain of a protein other than MYOCD.

Throughout the disclosure, expression of a polynucleotide may refer to any means known in the art to increase the expression of a gene of interest. In some embodiments, the gene of interest is encoded in the messenger RNA (mRNA). The mRNA may be synthetic or naturally occurring. In some embodiments, the mRNA is chemically modified in various ways known in the art. For example, modified RNAs may be used, such as described in Warren, L. et al. Cell Stem Cell 7:618-30 (2010); WO2014081507A1; WO2012019168; WO2012045082; WO2012045075; WO2013052523; WO2013090648; U.S. Pat. No. 9,572,896B2. In some embodiments, expression of the gene of interest is increased by delivery of a polynucleotide to a cell. In some embodiments, the polynucleotide encoding the gene of interest is delivered by a viral or non-viral vector. In some embodiments, the gene of interest is encoded in the DNA polynucleotide, optionally delivered by any viral or non-viral method known in the art. In some embodiments, the disclosure provides methods comprising contacting cells with a lipid nanoparticle comprising a DNA or mRNA encoding a gene of interest. In some embodiments, the methods of the disclosure comprise contacting cells with a virus comprising a DNA or RNA (e.g., a DNA genome, a negative-sense RNA genome, a positive-sense RNA genome, or a double-stranded RNA genome) encoding a gene of interest. In some embodiments, the virus is selected from a retrovirus, adenovirus, AAV, non-integrating lentiviral vector (LVV), and an integrating LVV. In some embodiments, the cells are transfected with a plasmid. In some embodiments, the plasmid comprises a polynucleotide encoding a reprogramming factor. In some embodiments, the plasmid comprises a transposon comprising a reprogramming factor.

In some embodiments, the reprogramming factors are provided as a polynucleotide encoding the one or more proteins of interest. In some embodiments, the polynucleotides are RNA, DNA, or mRNA polynucleotides. In some embodiments, the polynucleotides comprise a nucleic acid sequence that comprises at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the nucleotide sequence of human ASCL1 (SEQ ID NO: 2) across at least 100, 200, 300, 400, or 500 nucleotides. In some embodiments, the ASCL1 polynucleotide shares identity with any of the isoforms of ASCL1. In some embodiments, the ASCL1 polynucleotide encodes an ASCL1 protein that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of human ASCL1 (SEQ ID NO: 1).

In some embodiments, the compositions and methods of the disclosure provide iCM cells or recombinant virus or non-viral vectors comprising, or methods comprising administering, an MYOCD polynucleotide. In some embodiments, the MYOCD polynucleotide encodes a MYOCD protein that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of human MYOCD (SEQ ID NO: 3). In some embodiments, the MYOCD polynucleotide shares at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the nucleotide sequence of human MYOCD (SEQ ID NO: 4) across at least 100, 200, 300, 400, or 500 nucleotides. In some embodiments, the MYOCD polynucleotide shares identity with any of the isoforms of MYOCD.

In some embodiments, the engineered MYOCD protein is provided as a polynucleotide encoding the engineered MYOCD protein.

In some embodiments, the MYOCD polynucleotide encodes a MYOCD protein that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of an engineered MYOCD (e.g., SEQ ID NOs: 14).

In some embodiments, the MYOCD polynucleotide encodes a MYOCD protein that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of an engineered MYOCD (e.g., SEQ ID NOs: 15).

In some embodiments, the MYOCD polynucleotide encodes a MYOCD protein that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of an engineered MYOCD (e.g., SEQ ID NOs: 16).

In some embodiments, the polynucleotide encoding a protein of interest is a synthetic mRNA. Synthetic mRNAs provide the genetic information for making proteins of interest and can be chemically modified to avoid triggering an immune response. Zangi et al. (2013) Nature Biotech 31:898-907. Since mRNAs do not integrate into the host cell genome, the synthetic mRNA acts for a period of time and then disappears as the cell divides. In some embodiments the synthetic mRNAs are modified, for example, with pseudouridine and/or 5-methyl-cytidine, to reduce innate antiviral response to single-stranded RNA.

In some embodiments, the polynucleotides encoding the one or more proteins of interest may be codon-optimized or otherwise altered so long as the functional activity of the encoded gene is preserved. In some embodiments, the polynucleotides encode a modified or variant of the one or more genes of interest, including truncations, insertions, deletions, or fragments, so long as the functional activity of the encoded gene is preserved.

In some embodiments, the polynucleotides encoding the one or more proteins of interest are comprised in an expression cassette. In some embodiments, the expression cassette comprises one or more polynucleotides encoding one or more proteins of interest. For example, in some embodiments, the expression cassette comprises 2, 3, 4, 5, 6, 7, 8, 9, or 10 polynucleotides encoding 2, 3, 4, 5, 6, 7, 8, 9, or 10 genes of interest.

It will be appreciated that where two or more proteins of interest are to be expressed in a cell, one or polynucleotides or expression cassettes can be used. For example, a polycistronic expression cassette can be used wherein one expression cassette can comprise multiple polynucleotides expressing multiple proteins. In some embodiments, the polycistronic expression cassette comprises two or more polynucleotides in a single open reading frame, the polynucleotides linked together by the 2A region of aphthovirus foot-and-mouth disease virus (FMDV) polyprotein, such as described in Donnelly et al. J. Gen. Virol. 82:1013-15 (2001) and improvements thereof known in the art. The 2A region produces a ribosomal ‘skip’ from one codon to the next without the formation of a peptide bond. In some embodiments, the polynucleotide comprises an internal cleavage site, such that two or more peptides are generated by post-translational cleavage.

In some embodiments, multicistronic vectors of the present disclosure comprise a polynucleotide sequence encoding a plurality of polypeptides joined by linkers comprising peptides capable of inducing ribosome skipping or self-cleavage. In some embodiments, the linker comprises a 2A peptide. The term “2A peptide” as used herein refers to a class of ribosome skipping or self-cleaving peptides configured to generate two or more proteins from a single open reading frame. 2A peptides are 18-22 residue-long viral oligopeptides that mediate “cleavage” of polypeptides during translation in eukaryotic cells. “2A peptide” may refer to peptides with various amino acid sequences. In the present disclosure it will be understood that where a lentiviral vector comprises two or more 2A peptides, the 2A peptides may be identical to one another or different. Detailed methodology for design and use of 2A peptides is provided by Szymczak-Workman et al. Design and Construction of 2A Peptide-Linked Multicistronic Vectors. Cold Spring Harb. Protoc. 2012 Feb. 1; 2012(2):199-204. In the literature, 2A peptides are often referred to as self-cleaving peptides, but mechanistic studies have shown that the “self-cleavage” observed is actually a consequence of the ribosome skipping the formation of the glycyl-prolyl peptide bond at the C terminus of the 2A peptide. Donnelly et al. J Gen Virol. 2001 May; 82(Pt 5):1027-41. The present invention is not bound by theory or limited to any particular mechanistic understanding of 2A peptide function.

Exemplary 2A peptides include, without limitation, those listed in Table 4.

TABLE 4
Exemplary 2A peptides

| | Source | Nucleotide | Peptide |
|---|---|---|---|
| P2A | Porcine teschovirus-1 | GCC ACG AAC TTC TCT CTG | ATNFSLLKQAGDVEENPGP |
| | | TTA AAG CAA GCA GGA | (SEQ ID NO: 23) |
| | | GAC GTG GAA GAA AAC | |
| | | CCC GGT CCT | |
| | | (SEQ ID NO: 21) | |
| | | - or - | |
| | | GCT ACT AAC TTC AGC CTG | |
| | | CTG AAG CAG GCT GGA | |
| | | GAC GTG GAG GAG AAC | |
| | | CCT GGA CCT | |
| | | (SEQ ID NO: 22) | |
| T2A | Thoseaasigna virus | GAG GGC AGA GGA AGT | EGRGSLLTCGDVEENPGP |
| | | CTG CTA ACA TGC GGT GAC | (SEQ ID NO: 25) |
| | | GTC GAG GAG AAT CCT | |
| | | GGA CCT | |
| | | (SEQ ID NO: 24) | |
| E2A | Equine rhinitis A virus | CAG TGT ACT AAT TAT GCT | QCTNYALLKLAGDCESNPGP |
| | (ERAV) | CTC TTG AAA TTG GCT GGA | (SEQ ID NO: 27) |
| | | GAT GTT GAG AGC AAC CCT | |
| | | GGA CCT | |
| | | (SEQ ID NO: 26) | |
| F2A | Foot-and-mouth disease | GTG AAA CAG ACT TTG AAT | VKQTLNFDLLKLAGDVESNPGP |
| | virus (FMDV) | TTT GAC CTT CTC AAG TTG | (SEQ ID NO: 29) |
| | | GCG GGA GAC GTG GAG | |
| | | TCC AAC CCT GGA CCT | |
| | | (SEQ ID NO: 28) | |

Optionally, one or more of the linkers further comprises a sequence encoding the residues Gly-Ser-Gly, which is in some embodiments N-terminal to the 2A peptide. N-terminal to the 2A peptide means that the sequence encoding the residues is upstream to the sequence encoding the 2A peptide. Generally, the Gly-Ser-Gly motif will be immediately N-terminal to the 2A peptide or 1 to 10 other amino acid residues are inserted between the motif and the 2A peptide. In some embodiments, the polynucleotide sequence encoding this motif is GGA AGC GGA. As with any peptide-encoding polynucleotide, the nucleotide sequence may be altered without changing the encoded peptide sequence. Substitution of amino acid residues is within the skill of those in the art, and it will be understood that the term 2A peptide refers to variants of the foregoing that retain the desired skipping/self-cleavage activity but, optionally, have 1, 2, 3, 4, 5, 6, 7, 8, 9, 10 or more substitutions relative to the reference 2A peptide sequence. Examplary 2A peptides are described in Kim et al. PLOS ONE 6(4): e18556. In some embodiments, two or more different 2A peptides are used in the same construct. Varied 2A peptides have been reported to result in improved expression. See Liu et al. Sci Rep. 2017; 7:2193

In some embodiments, the disclosure provides an expression cassette comprising, in 5′ to 3′ order, a promoter, a polynucleotide encoding MYOCD-2A-ASCL1, and a polyadenylation sequence. In some embodiments, the expression cassette comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 35. In some embodiments, the disclosure provides a recombinant AAV (rAAV) comprising the expression cassette, a transfer plasmid comprising the expression cassette, or a rAAV particle comprising the expression cassette. In some embodiments, the rAAV comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 35. In some embodiments, the disclosure provides a recombinant lentivirus (rLV) comprising the expression cassette, a transfer plasmid comprising the expression cassette, or a rLV particle comprising the expression cassette. In some embodiments, the rLV comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 35.

In some embodiments, the disclosure provides an expression cassette comprising, in 5′ to 3′ order, a promoter, a polynucleotide encoding MyΔ3-2A-ASCL1, and a polyadenylation sequence. In some embodiments, the expression cassette comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 37. In some embodiments, the disclosure provides a rAAV comprising the expression cassette, a transfer plasmid comprising the expression cassette, or a rAAV particle comprising the expression cassette. In some embodiments, the rAAV comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 37. In some embodiments, the disclosure provides a rLV comprising the expression cassette, a transfer plasmid comprising the expression cassette, or a rLV particle comprising the expression cassette. In some embodiments, the rLV comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 37.

In some embodiments, the disclosure provides an expression cassette comprising, in 5′ to 3′ order, a promoter, a polynucleotide encoding ASCL1-2A-MYOCD, and a polyadenylation sequence. In some embodiments, the expression cassette comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 39. In some embodiments, the disclosure provides a rAAV comprising the expression cassette, a transfer plasmid comprising the expression cassette, or a rAAV particle comprising the expression cassette. In some embodiments, the rAAV comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 39. In some embodiments, the disclosure provides a rLV comprising the expression cassette, a transfer plasmid comprising the expression cassette, or a rLV particle comprising the expression cassette. In some embodiments, the rLV comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 39.

In some embodiments, the disclosure provides an expression cassette comprising, in 5′ to 3′ order, a promoter, a polynucleotide encoding ASCL1-2A-MyΔ3, and a polyadenylation sequence. In some embodiments, the expression cassette comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41. In some embodiments, the disclosure provides a rAAV comprising the expression cassette, a transfer plasmid comprising the expression cassette, or a rAAV particle comprising the expression cassette. In some embodiments, the rAAV comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41. In some embodiments, the disclosure provides a rLV comprising the expression cassette, a transfer plasmid comprising the expression cassette, or a rLV particle comprising the expression cassette. In some embodiments, the rLV comprises a polynucleotide at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41.

In some embodiments, the disclosure provides an expression cassette comprising a polynucleotide encoding a protein sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 57-64 (e.g., any one of MYOCD-2A-ASCL1, My43-2A-ASCL1, ASCL1-2A-MYOCD, and ASCL1-2A-MyΔ3).

In some embodiments, the one or more reprogramming factors comprise one or more microRNAs. MicroRNAs useful as reprogramming factors include miR-133a-2, miR-133a-1, miR-19b-2, miR-19b-1, miR-326, miR-1-1, miR-1298, miR-133b, miR-1-2, miR-92a-2, miR-20b, miR-20a, miR-141, miR-155, miR-17, hsa-let-7c, miR-202, miR-200a, miR-206, miR-509-1, miR-509-2, miR-124-3, miR-124-2, miR-378a, miR-378e, miR-378h, miR-378i, miR-137, miR-671, miR-24-1, miR-182, miR-302d, miR-96, miR-30c-2, and miR-146b.

In some embodiments, the microRNA is selected from the group consisting of miR-19b-1, miR-19b-2, miR-137, miR-133a-2, miR-671, miR-24-1, miR-182, miR-302d, miR-96, miR-30c-2, miR-146b, and miR-133a-2.

In some embodiments, the microRNA is selected from the group consisting of miR-133a-2, miR-133a-1, miR-19b-2, miR-19b-1, miR-326, miR-1-1, miR-1298, miR-133b, miR-1-2, miR-92a-2, miR-20b, miR-20a, miR-141, miR-155, miR-17, hsa-let-7c, miR-202, miR-200a, miR-206, miR-509-1, miR-509-2, miR-124-3, miR-124-2, miR-378a, miR-378e, miR-378h, miR-378i, miR-137, miR-671, miR-24-1, miR-182, miR-302d, miR-96, miR-30c-2, and miR-146b.

In some embodiments, the microRNA is selected from the group consisting of miR-133a-2, miR-133a-1, miR-19b-2, miR-19b-1, miR-326, miR-1-1, miR-1298, miR-133b, miR-1-2, miR-92a-2, miR-20b, miR-20a, miR-141, miR-155, miR-17, hsa-let-7c, miR-202, miR-200a, miR-206, miR-509-1, miR-509-2, miR-124-3, miR-124-2, miR-378a, miR-378e, miR-378h, and miR-378i.

In some embodiments, the microRNA is selected from the group consisting of miR-133a-2, miR-133a-1, miR-19b-2, miR-19b-1, miR-326, miR-1-1, miR-1298, miR-133b, miR 2, miR-92a-2, miR-20b, miR-20a, miR-141, miR-155, miR-17, hsa-let-7c, miR-202, miR-200a, miR-206, miR-509-1, miR-509-2, miR-124-3, miR-124-2, miR-378a, miR-378e, miR-378h, miR-378i, miR-137, miR-671, miR-24-1, miR-182, miR-302d, miR-96, miR-30c-2, and miR-146b.

In some embodiments, two microRNAs are combined with MYOCD and/or ASCL1 to induce reprogramming of differentiated cells (e.g., fibroblasts) to cardiomyocytes. Possible combinations of microRNAs include any one of miR-133a-2, miR-133-al, miR-19b-2, miR-19b-1, miR-326, miR-1-1, miR-1298, miR-133-b, miR-1-2, miR-20-b, and miR-20-a in a 5′ position followed by any one of miR-133a-2, miR-133-al, miR-19b-2, miR-19b-1, miR-326, miR-1-1, miR-1298, miR-133-b, miR-1-2, miR-20-b, and miR-20-a in a 3′ position. In some embodiments, multiple miRNAs are combined, such as at least 3, 4, or 5 miRNAs. Multiple copies of the same miRNA can be used, such as 1, 2, 3, 4, 5 or more copies of the same microRNA.

The microRNA of interest may be provided by any means, including, without limitation, as an shRNA, siRNA, or microRNA mimetic (optionally including modifications such a phosphothiolate backbone, locked nucleic acids, and cholesterol modifications). In some embodiments, the microRNA of interest is expressed from a polynucleotide encoding the microRNA as a pre-miRNA. The polynucleotide encoding the microRNA is generally operatively linked to a promoter. The microRNA can be expressed on its own transcript or on a shared transcript with one or more other factors (e.g. polynucleotides encoding proteins of interest). In some embodiments, the MYOCD polynucleotide and/or ASCL1 polynucleotide is arranged with the polynucleotide encoding a microRNA in a vector such that the microRNA and the MYOCD and/or ASCL1 are expressed from the same transcript. In some embodiments, the pre-miRNA sequence is 5′ to the protein coding sequence (e.g. MYOCD, ASCL1, MYOCD-2A-ASCL1, or ASCL1-2A-MYOCD). In some embodiments, the pre-miRNA sequence is 3′ to the protein coding sequence (e.g. MYOCD, ASCL1, MYOCD-2A-ASCL1, or ASCL1-2A-MYOCD). The polynucleotide encoding the microRNA may be inserted in a 5′ or 3′ untranslated region (UTR). The polynucleotide encoding the microRNA may be inserted in intron.

Illustrative pri-miRNA sequences useful in the compositions and methods of the present disclosure are provided in Table 1. In some embodiments, 1, 2, 3, 4, or more substitutions and/or insertions in the stem or loop of the pri-miRNA are tolerated. In some embodiments, the polynucleotide comprises a sequence at least 95%, 96%, 97%, 98%, 99%, or 100% identical to a native microRNA (e.g. a native human microRNA). In some embodiments, the polynucleotide comprises a sequence at least 95%, 96%, 97%, 98%, 99%, or 100% identical to any one of SEQ ID NOs: 65-99. In some embodiments, the polynucleotide comprises a sequence at least 95%, 96%, 97%, 98%, 99%, or 100% identical to any one of SEQ ID NOs: 100-134.

In some embodiments, the polynucleotide comprises a sequence at least 95%, 96%, 97%, 98%, 99%, or 100% identical to any one of hsa-pri-miR-133a-2, hsa-pri-miR-133a-1, hsa-pri-miR-19b-2, hsa-pri-miR-19b-1, hsa-pri-miR-326, hsa-pri-miR-1-1, hsa-pri-miR-1298, hsa-pri-miR-133b, hsa-pri-miR-1-2, hsa-pri-miR-92a-2, hsa-pri-miR-20b, hsa-pri-miR-20a, hsa-pri-miR-141, hsa-pri-miR-155, hsa-pri-miR-17, hsa-pri-let-7c, hsa-pri-miR-202, hsa-pri-miR-200a, hsa-pri-miR-206, hsa-pri-miR-509-1, hsa-pri-miR-509-2, hsa-pri-miR-124-3, hsa-pri-miR-124-2, hsa-pri-miR-378a, hsa-pri-miR-378e, hsa-pri-miR-378h, hsa-pri-miR-378i, hsa-pri-miR-137, hsa-pri-miR-671, hsa-pri-miR-24-1, hsa-pri-miR-182, hsa-pri-miR-302d, hsa-pri-miR-96, hsa-pri-miR-30c-2, and hsa-pri-miR-146b.

In some embodiments, the polynucleotide comprises a sequence at least 95%, 96%, 97%, 98%, 99%, or 100% identical to any one of hsa-MIR-133a-2, hsa-MIR-133a-1, hsa-MIR-19b-2, hsa-MIR-19b-1, hsa-MIR-326, hsa-MIR-1-1, hsa-MIR-1298, hsa-MIR-133b, hsa-MIR-1-2, hsa-MIR-92a-2, hsa-MIR-20b, hsa-MIR-20a, hsa-MIR-141, hsa-MIR-155, hsa-MIR-17, hsa-let-7c, hsa-MIR-202, hsa-MIR-200a, hsa-MIR-206, hsa-MIR-509-1, hsa-MIR-509-2, hsa-MIR-124-3, hsa-MIR-124-2, hsa-MIR-378a, hsa-MIR-378e, hsa-MIR-378h, hsa-MIR-378i, hsa-MIR-137, hsa-MIR-671, hsa-MIR-24-1, hsa-MIR-182, hsa-MIR-302d, hsa-MIR-96, hsa-MIR-30c-2, and hsa-MIR-146b.

### MicroRNA Binding Sites

In an aspect, the disclosure provides a vector, comprising a polynucleotide comprising a polynucleotide sequence encoding one or more transgenes and a microRNA binding site for a microRNA, wherein the microRNA binding site is operatively linked to the polynucleotide sequence encoding the one or more transgenes, and wherein the microRNA is expressed at a higher level in cardiomyocytes or cardiomyocyte progenitors compared to cardiac fibroblasts. A microRNA binding site may be operatively linked to the polynucleotide by inserting the microRNA binding site in the sequence encoding the mRNA transcript encoding the one or more transgenes. The insertion site may be in a coding or non-coding region. An insertion in a coding region may be generated by selecting a tolerated position with the encoded protein (e.g. a loop) and ensuring the binding site is inserted using an in-frame insertion. The binding site may be inserted into 3′ untranslated region (3′ UTR), i.e. between the end of the last transgene and the polyadenylation site. The binding site may also be inserted between a pair of transgenes or in the 5′ UTR.

The microRNA binding site may also be indirectly coupled to one or more transgenes via a gene regulatory circuit. For example, the microRNA binding site may be operatively linked to an enhancer of transcription, an enhancer of translation, or a co-factor. In some embodiments, the vector encodes multiple mRNA transcripts. The microRNA binding site may be provided in some or all of these transcripts. For example, microRNA binding sites may be operatively linked to a polynucleotide encoding a mRNA transcript encoding ASCL1, to a polynucleotide encoding a mRNA transcript encoding a myocardin, or to both.

In some embodiments, the microRNA binding site promotes specific repression of expression of the one or more transgenes in a cardiomyocyte or cardiomyocyte progenitor compared to a cardiac fibroblast.

In some embodiments, the microRNA is expressed at a lower level in cardiac fibroblasts and/or is expressed at a lower level in cardiac fibroblasts treated with the cardiomyocytes reprogramming factor for about 7 days or less, about one week or less, about two weeks or less, about three weeks or less, or about four weeks or less, compared to a level of expression of the microRNA in cardiomyocytes and/or a level of expression of the microRNA in cardiac fibroblasts treated with the cardiomyocytes reprogramming factor for more than about 7 days, more than about one week, more than about two weeks, more than about three weeks, or more than about four weeks.

In some embodiments, the microRNA is miR-208. In some embodiments, the microRNA is miR-1. In some embodiments, the microRNA is miR-133. In some embodiments, the microRNA is miR-208a. In some embodiments, the microRNA is miR-208b.

In some embodiments, the microRNA is miR-208b-3p. In some embodiments, the microRNA binding site shares >70% identity to ACAAACCTTTTGTTCGTCTTAT (SEQ ID NO: 135) and no mismatches in the underlined seed region comprising the sequence CGTCTTA. In some embodiments, the microRNA binding site is AAAATATATGTAATCGTCTTAA (SEQ ID NO: 136).

In some embodiments, the microRNA binding site is ACAAACCTTTTGTTCGTCTTAT (SEQ ID NO: 135). In some embodiments, the microRNA binding site is TGAAACCTTTTGTTCGTCTTAT (SEQ ID NO: 137). In some embodiments, the polynucleotide comprises at least two microRNA binding sites for the microRNA. In some embodiments, the polynucleotide comprises at least three microRNA binding sites for the microRNA.

In some embodiments, the polynucleotide comprises at least four microRNA binding sites for the microRNA. In some embodiments, the polynucleotide comprises at least five microRNA binding sites for the microRNA. In some embodiments, the polynucleotide comprises at least six microRNA binding sites for the microRNA. In some embodiments, the polynucleotide comprises at most six microRNA binding sites for the microRNA. In some embodiments, the polynucleotide comprises one, two, three, four, five, or six microRNA binding sites for the microRNA. In some embodiments, the one or more transgenes comprises one or more cardiomyocyte reprogramming factors. In some embodiments, the one or more cardiomyocyte reprogramming factors comprises two or more of MYOCD, ASCL1, GATA4, MEF2C, TBXS, miR-133, and MESP1. In some embodiments, the one or more cardiomyocyte reprogramming factors comprises three or more of MYOCD, ASCL1, GATA4, MEF2C, TBXS, miR-133, and MESP1. In some embodiments, the one or more cardiomyocyte reprogramming factors comprises four or more of MYOCD, ASCL1, GATA4, MEF2C, TBXS, miR-133, and MESP1. In some embodiments, the one or more cardiomyocyte reprogramming factors comprises five or more of MYOCD, ASCL1, GATA4, MEF2C, TBXS, miR-133, and MESP1. In some embodiments, the one or more cardiomyocyte reprogramming factors comprise MYOCD and ASCL1. In some embodiments, the polynucleotide sequence encodes a MYOCD-2A-ASCL1 protein.

The disclosure is not limited to vectors encoding cardiomyocyte reprogramming factors. Indeed the vectors described herein may deliver other genes for which expression in a selected non-target cell is undesirable. For example and without limitation, the vectors may be used to deliver gene therapies to cells other than cardiomyocytes. The target cells may be cardiac fibroblasts or any other cell type. In some embodiments, the vectors of the disclosure are useful to transducing cells in organs other than the heart (e.g., lung, brain, liver, muscle, etc.).

In some embodiments, the MYOCD comprises an internal deletion. Illustrative MYOCD genes are provided in International Patent Application No. PCT/US2019/049150, which is incorporated by reference herein in its entirety.

In some embodiments, the polynucleotide comprises, in 5′ to 3′ order, a promoter, a sequence encoding the MYOCD and the ASCL1, the microRNA binding site, and a polyadenylation sequence.

In some embodiments, the polynucleotide comprises a sequence encoding miR-133.

In some embodiments, the vector is a viral vector. In some embodiments, the viral vector is an adeno-associated virus (AAV) vector, retroviral vector, lentiviral vector, adenoviral vector, herpes simplex virus vector, etc. In some embodiments, the AAV vector is an AAV9 vector. In some embodiments, the AAV vector is an AAVS vector.

In another aspect, the disclosure provides a method for reprogramming cardiac fibroblasts into cardiomyocytes, comprising a) selecting a microRNA specifically expressed in induced cardiomyocytes by treating cardiac fibroblasts with an effective amount of a composition that induces reprogramming of cardiac fibroblasts to cardiomyocytes and measuring the expression of one or more microRNAs in the cardiac fibroblasts, wherein the selected microRNA is expressed in the cardiac fibroblasts only after a predetermined time; b) generating a vector comprising a polynucleotide comprising one or more microRNA binding sites for the selected microRNA operatively linked to a polynucleotide encoding one or more cardiomyocyte reprogramming factors; and c) contacting a cardiac fibroblast with an effective amount of the vector.

In some embodiments, the microRNA binding site represses expression of the one or more cardiomyocyte reprogramming factors in cardiomyocyte cells. In some embodiments, the microRNA binding site represses expression of the one or more cardiomyocyte reprogramming factors in skeletal muscle cells. In some embodiments, the microRNA binding site represses expression of the one or more cardiomyocyte reprogramming factors in cardiomyocyte progenitor cells.

In some embodiments, the microRNA is miR-208. In some embodiments, the microRNA is miR-1. In some embodiments, the microRNA is miR-133. In some embodiments, the microRNA is miR-208a. In some embodiments, the microRNA is miR-208b.

In some embodiments, the microRNA is miR-208b-3p. In some embodiments, the microRNA binding site shares >70%, >75%, >%80, >90%, >95%, >99%, or >100% identity to ACAAACCTTTTGTTCGTCTTAT (SEQ ID NO: 135) and no mismatches in the underlined seed region comprising the sequence CGTCTTA. In some embodiments, the microRNA binding site is AAAATATATGTAATCGTCTTAA (SEQ ID NO: 136). In some embodiments, the microRNA binding site is ACAAACCTTTTGTTCGTCTTAT (SEQ ID NO: 135). In some embodiments, the microRNA binding site is TGAAACCTTTTGTTCGTCTTAT (SEQ ID NO: 137). In some embodiments, the polynucleotide comprises at least two microRNA binding sites for the microRNA.

In some embodiments, the AAV vector comprises a 3′ UTR that shares >70%, >75%, >%80, >90%, >95%, >99%, or >100% identity to:

(SEQ ID NO: 138)
ACAAACCTTTTGTTCGTCTTATAAAACAAACCTTTTGTTCGTCTTATAAA
ACAAACCTTTTGTTCGTCTTATAAAACAAACCTTTTGTTCGTCTTAT.

In some embodiments, the AAV vector comprises a 3′ UTR that shares >70%, >75%, >%80, >90%, >95%, >99%, or >100% identity to:

(SEQ ID NO 139)
ACAAACCTTTTGTTCGTCTTATAAAACAAACCTTTTGTTCGTCTTAT.

In some embodiments, the AAV vector comprises a 3′ UTR that shares >70%, >75%, >%80, >90%, >95%, >99%, or >100% identity to:

(SEQ ID NO: 140)
AAAATATATGTAATCGTCTTAAAAAAAATATATGTAATCGTCTTAAAAAA
AATATATGTAATCGTCTTAAAAAAAATATATGTAATCGTCTTAA.

In some embodiments, the polynucleotide comprises at least four microRNA binding sites for the microRNA. In some embodiments, the polynucleotide comprises at most six microRNA binding sites for the microRNA. In some embodiments, the polynucleotide comprises four microRNA binding sites for the microRNA. In another aspect, the disclosure provides method for reprogramming a cardiac fibroblast into a cardiomyocyte cell, comprises contacting the cardiac fibroblast with an effective amount of the vector of any one of claims 1 to 28.

In some embodiments, the method induces expression of at least one marker of cardiomyocyte phenotype in the cardiac fibroblast. In some embodiments, the microRNA binding site is a microRNA binding site for miR-1, miR-133, miR-208a, miR-208b, and/or miR-208b-3p. In some embodiments, the microRNA binding site is a microRNA binding site for miR-1. In some embodiments, the microRNA binding site is a microRNA binding site for miR-133.

In some embodiments, the microRNA binding site is a microRNA binding site for miR-208a. In some embodiments, the microRNA binding site is a microRNA binding site for miR-208b. In some embodiments, the microRNA binding site is a microRNA binding site for miR-208b-3p. In some embodiments, the polynucleotide comprises a sequence encoding miR-133.

In some embodiments, the heart failure is due to myocardial infarction. In some embodiments, the heart failure is heart failure with reduced ejection fraction (HFrEF). In some embodiments, the method increases ejection fraction in the subject compared to the subject before administration.

In some embodiments, the method increases ejection fraction in the subject compared to an untreated control subject. In some embodiments, the method increases ejection fraction in the subject to at least about 28%, 29%, 30%, 31%, or 32%. In some embodiments, ejection fraction is assessed after a predetermined time, optionally eight weeks after administration of the AAV vector. In some embodiments, the method decreases scar tissue formation in the subject compared to the subject before administration. In some embodiments, the method decreases scar tissue formation in the subject compared to an untreated control subject. In some embodiments, the method decreases scar tissue formation in the subject to at most about 10%, 11%, 12%, 13%, 14%, 15%, 16%, 17%, 18%, 19%, or 20%. In some embodiments, scar tissue formation is assessed after a predetermined time, optionally eight weeks after administration of the AAV vector.

### III. Vectors

In some embodiments, the reprogramming factors employed to reprogram cells to the cardiac lineage can be introduced into a selected cell or a selected population of cells by a vector. In some embodiments, the vector is a nucleic acid vector, such as a plasmids (e.g., DNA plasmids or RNA plasmids), transposons, cosmids, bacterial or yeast artificial chromosomes, or viral vectors. In some embodiments, the vector is a non-nucleic acid vector, such as a nanoparticle. In some embodiments, the vectors described herein comprise a peptide, such as cell-penetrating peptides or cellular internalization sequences. Cell-penetrating peptides are small peptides that are capable of translocating across plasma membranes. Exemplary cell-penetrating peptides include, but are not limited to, Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynBl, Pep-7, I-IN-1, BGSC (Bis-Guanidinium-Spermidine-Cholesterol, and BGTC (Bis-Guanidinium-Tren-Cholesterol).

Techniques in the field of recombinant genetics can be used for such recombinant expression. Basic texts disclosing general methods of recombinant genetics include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)). In some embodiments, the vectors do not contain a mammalian origin of replication. In some embodiments, the expression vector is not integrated into the genome and/or is introduced via a vector that does not contain a mammalian origin of replication.

In some cases, the expression vector(s) encodes or comprises, in addition to one or more reprogramming factors, a marker gene that facilitates identification or selection of cells that have been transfected, transduced or infected. Examples of marker genes include, but are not limited to, genes encoding fluorescent proteins, e.g., enhanced green fluorescent protein, Ds-Red (DsRed: Discosoma sp. red fluorescent protein (RFP); Bevis et al. (2002) Nat. Biotechnol. 20(11):83-87), yellow fluorescent protein, mCherry, and cyanofluorescent protein; and genes encoding proteins conferring resistance to a selection agent, e.g., a neomycin resistance gene, a puromycin resistance gene, a blasticidin resistance gene, and the like.

In one embodiment, the expression vector further comprises a suicide gene. Expression of the suicide gene may be regulated by the same or different promoter that expresses at least one proliferation and/or cell cycle reentry factor polypeptide-encoding nucleotide. A suicide gene is one that allows for negative selection of the cells. In the methods described herein, a suicide gene is used as a safety system, allowing the cells expressing the gene to be killed by introduction of a selective agent. This is desirable in case the recombinant gene causes a mutation leading to uncontrolled cell growth. A number of suicide gene systems have been identified, including the herpes simplex virus thymidine kinase (tk or TK) gene, the cytosine deaminase gene, the varicella-zoster virus thymidine kinase gene, the nitroreductase gene, the Escherichia coli (E. coli) gpt gene, and the E. coli Deo gene (also see, for example, Yazawa K, Fisher W E, Brunicardi F C: Current progress in suicide gene therapy for cancer. World J. Surg. (2002) 26(7):783-9). In one embodiment, the suicide gene is the TK gene. In one aspect, the TK gene is a wild- type TK gene. In other aspects, the TK gene is a mutated form of the gene, e.g., sr23tk. Cells expressing the TK protein can be killed using ganciclovir. In another embodiment, the nucleic acid encoding the tetracycline activator protein and the suicide gene are regulated by one promoter.

### A. Nucleic Acid Vectors

### 1. Viral Vectors

Suitable viral vectors include, but are not limited to, viral vectors (e.g. viral vectors based on vaccinia virus; poliovirus; adenovirus (e.g., Li et al. (1994) Invest Opthalmol Vis Sci 35:2543-2549; Borras et al. (1999) Gene Ther 6:515-524; Li and Davidson, (1995) Proc. Natl. Acad. Sci. 92:7700-7704; Sakamoto et al. (1999) Hum Gene Ther 5: 1088-1097; WO 94/12649; WO 93/03769; WO 93/19191; WO 94/28938; WO 95/11984 and WO 95/00655); adeno-associated virus (e.g., Ali et al. (1998) Hum Gene Ther 9(0:81-86, 1998, Flannery et al. (1997) Proc. Natl. Acad. Sci. 94:6916-6921; Bennett et al. (1997) Invest Opthalmol Vis Sci 38:2857-2863; Jomary et al. (1997) Gene Ther 4:683-690; Rolling et al. (1999), Hum Gene Ther 10:641-648; Ali et al. (1996) Hum Mol Genet. 5:591-594; WO 93/09239, Samulski et al. (1989) J. Vir. 63 :3822-3828; Mendelson et al. (1988) Virol. 166: 154-165; and Flotte et al. (1993) Proc. Natl. Acad. Sci. 90: 10613-10617; SV40; herpes simplex virus; human immunodeficiency virus (e.g., Miyoshi et al. (1997) Proc. Natl. Acad. Sci. 94: 10319-10323; Takahashi et al. (1999) J Virol 73 :7812-7816); a retroviral vector (e.g., Murine-Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available. The following vectors are provided by way of example; for eukaryotic cells: pXT1, pSG5 (Stratagene), pSVK3, pBPV, pMSG, pSVLSV40 (Pharmacia), and pAd (Life Technologies). However, any other vector may be used so long as it is compatible with the cells of the present disclosure.

The ability of certain viruses to infect cells or enter cells via receptor-mediated endocytosis, and express viral genes stably and efficiently have made them attractive candidates for the transfer of foreign nucleic acids into cells (e.g., mammalian cells). Viral vectors can include control sequences such as promoters for expression of the polypeptide of interest. Although many viral vectors integrate into the host cell genome, if desired, the segments that allow such integration can be removed or altered to prevent such integration. Moreover, in some embodiments, the vectors do not contain a mammalian origin of replication. Non-limiting examples of virus vectors are described below that can be used to deliver nucleic acids encoding a transcription factor into a selected cell. In some embodiments, the viral vector is derived from a replication-deficient virus.

In general, other useful viral vectors are based on non-cytopathic eukaryotic viruses in which non-essential genes have been replaced with the polypeptide of interest. Non-cytopathic viruses include certain retroviruses, the life cycle of which involves reverse transcription of genomic viral RNA into DNA with subsequent proviral integration into host cellular DNA. In general, the retroviruses are replication-deficient (e.g., capable of directing synthesis of the desired transcripts, but incapable of manufacturing an infectious particle). Such genetically altered retroviral expression vectors have general utility for the high-efficiency transduction of polynucleotide in vivo.

In some embodiments, a polynucleotide encoding a reprogramming factor can be housed within an infective virus that has been engineered to express a specific binding ligand. The virus particle will thus bind with specificity to the cognate receptors of the target cell and deliver the contents to the cell. In some embodiments, the virus is modified to impart particular viral tropism, e.g., the virus preferentially infects fibroblasts, heart cells, or more particularly cardiac fibroblasts (CFs). For AAV, capsid proteins can be mutated to alter the tropism of the viral vector. For lentivirus, tropism can be modified by using different envelope proteins; this is known as “pseudotyping.”

a. Retroviral Vectors

In some embodiments, the viral vector is a retroviral vector. Retroviruses can integrate their genes into the host genome, transfer a large amount of foreign genetic material, infect a broad spectrum of species and cell types, and can be packaged in special cell-lines (Miller et al., Am. J. Clin. Oncol., 15(3):216-221, 1992). In some embodiments, a retroviral vector is altered so that it does not integrate into the host cell genome.

The recombinant retrovirus may comprise a viral polypeptide (e.g., retroviral env) to aid entry into the target cell. Such viral polypeptides are well-established in the art, for example, U.S. Pat. No. 5,449,614. The viral polypeptide may be an amphotropic viral polypeptide, for example, amphotropic env, which aids entry into cells derived from multiple species, including cells outside of the original host species. The viral polypeptide may be a xenotropic viral polypeptide that aids entry into cells outside of the original host species. In some embodiments, the viral polypeptide is an ecotropic viral polypeptide, for example, ecotropic env, which aids entry into cells of the original host species.

Examples of viral polypeptides capable of aiding entry of retroviruses into cells include, but are not limited to: MMLV amphotropic env, MMLV ecotropic env, MMLV xenotropic env, vesicular stomatitis virus-g protein (VSV-g), HIV-1 env, Gibbon Ape Leukemia Virus (GALV) env, RD114, FeLV-C, FeLV-B, MLV 10A1 env gene, and variants thereof, including chimeras. Yee et al. (1994) Methods Cell Biol, Pt A:99-1 12 (VSV-G); U.S. Pat. No. 5,449,614. In some cases, the viral polypeptide is genetically modified to promote expression or enhanced binding to a receptor.

The retroviral construct may be derived from a range of retroviruses, e.g., MMLV, HIV-1, SIV, FIV, or another retrovirus described herein. The retroviral construct may encode all viral polypeptides necessary for more than one cycle of replication of a specific virus. In some cases, the efficiency of viral entry is improved by the addition of other factors or other viral polypeptides. In other cases, the viral polypeptides encoded by the retroviral construct do not support more than one cycle of replication, e.g., U.S. Pat. No. 6,872,528. In such circumstances, the addition of other factors or other viral polypeptides can help facilitate viral entry. In an exemplary embodiment, the recombinant retrovirus is HIV-1 virus comprising a VSV-g polypeptide, but not comprising a HIV-1 env polypeptide.

The retroviral construct may comprise: a promoter, a multi-cloning site, and/or a resistance gene. Examples of promoters include but are not limited to CMV, SV40, EF1a, β-actin; retroviral LTR promoters, and inducible promoters. The retroviral construct may also comprise a packaging signal (e.g., a packaging signal derived from the MFG vector; a psi packaging signal). Examples of some retroviral constructs known in the art include but are not limited to: pMX, pBabeX or derivatives thereof. Onishi et al. (1996) Experimental Hematology, 24:324-329. In some cases, the retroviral construct is a self-inactivating lentiviral vector (SIN) vector. Miyoshi et al. (1998) J. Virol 72(10):8150- 8157. In some cases, the retroviral construct is LL-CG, LS-CG, CL-CG, CS-CG, CLG or MFG. Miyoshi et al. (1998) J. Virol 72(10):8150-8157; Onishi et al. (1996) Experimental Hematology, 24:324-329; Riviere et al. (1995) Proc. Natl. Acad. Sci., 92:6733-6737.

A retroviral vector can be constructed by inserting a nucleic acid (e.g., one encoding a polypeptide of interest or an RNA) into the viral genome in the place of some viral sequences to produce a virus that is replication-defective. To produce virions, a packaging cell line containing the gag, pol, and env genes, but without the LTR and packaging components, is constructed (Mann et al., Cell 33:153-159, 1983). When a recombinant plasmid containing a cDNA, together with the retroviral LTR and packaging sequences is introduced into a special cell line (e.g., by calcium phosphate precipitation), the packaging sequence allows the RNA transcript of the recombinant plasmid to be packaged into viral particles, which are then secreted into the culture media (Nicolas and Rubinstein, In: Vectors: A survey of molecular cloning vectors and their uses, Rodriguez and Denhardt, eds., Stoneham: Butterworth, pp. 494-513, 1988; Temin, In: Gene Transfer, Kucherlapati (ed.), New York: Plenum Press, pp. 149-188, 1986; Mann et al., Cell, 33:153-159, 1983). The media containing the recombinant retroviruses is then collected, optionally concentrated, and used for gene transfer. Retroviral vectors are able to infect a broad variety of cell types. However, integration and stable expression typically involves the division of host cells (Paskind et al., Virology, 67:242-248, 1975).

b. Adenoviral Vectors

In some embodiments, the viral vector is an adenoviral vector. The genetic organization of adenovirus includes an approximate 36 kb, linear, double-stranded DNA virus, which allows substitution of large pieces of adenoviral DNA with foreign sequences up to 7 kb (Grunhaus et al., Seminar in Virology 200(2):535-546, 1992)). Reprogramming factors may be introduced into the cell using adenovirus assisted transfection. Increased transfection efficiencies have been reported in cell systems using adenovirus coupled systems (Kelleher and Vos, Biotechniques, 17(6):1110-7, 1994; Cotten et al., Proc Natl Acad Sci USA, 89(13):6094-6098, 1992; Curiel, Nat Immun, 13(2-3):141-64, 1994.).

c. Adeno-Associated Viral (AAV) Vectors

In some embodiments, the viral vector is an AAV vector. AAV is an attractive vector system as it has a high frequency of integration and it can infect non-dividing cells, thus making it useful for delivery of polynucleotides into mammalian cells, for example, in tissue culture (Muzyczka, Curr Top Microbiol Immunol, 158:97-129, 1992) or in vivo. Details concerning the generation and use of rAAV vectors are described in U.S. Pat. Nos. 5,139,941 and 4,797,368, each incorporated herein by reference in its entirety.

AAV is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV- 10, AAV-11, AAV- 12, AAV-13 and AAV rh74. Production of pseudotyped rAAV is disclosed in, for example, WO 01/83692. Other types of rAAV variants, for example rAAV with capsid mutations, are also contemplated. The nucleotide sequences of the genomes of various AAV serotypes are known in the art. AAV vectors of the present disclosure include AAV vectors of serotypes AAV1, AAV2, AAV4, AAVS, AAV6, AAV7, AAV8, AAV9, AAV10, AAV39, AAV43, AAV.rh74, and AAV.rh8. Illustrative AAV vectors are provided in U.S. Pat. Nos. 7,105,345; 15/782,980; 7,259,151; 6,962,815; 7,718,424; 6,984,517; 7,718,424; 6,156,303; 8,524,446; 7,790,449; 7,906,111; 9,737,618; U.S. application Ser. No. 15/433,322; U.S. Pat. No. 7,198,951, each of which is incorporated by reference in its entirety.

In some embodiments, the AAV expression vector is pseudotyped to enhance targeting. To promote gene transfer and sustain expression in fibroblasts, AAV5, AAV7, and AAV8, may be used. In some cases, the AAV2 genome is packaged into the capsid of producing pseudotyped vectors AAV2/5, AAV2/7, and AAV2/8 respectively, as described in Balaji et al. J Surg Res. 2013 September; 184(1):691-698. In some embodiments, an AAV9 may be used to target expression in myofibroblast-like lineages, as described in Piras et al. Gene Therapy 23:469-478 (2016). In some embodiments, AAV1, AAV6, or AAV9 is used, and in some embodiments, the AAV is engineered, as described in Asokari et al. Hum Gene Ther. 2013 Nov; 24(11): 906-913; Pozsgai et al. Mol Ther. 2017 Apr. 5; 25(4): 855-869; Kotterman, M. A. and D. V. Schaffer (2014) Engineering Adeno-Associated Viruses for Clinical Gene Therapy. Nature Reviews Genetics, 15:445-451; and US20160340393A1 to Schaffer et al. In some embodiments, the viral vector is AAV engineered to increase target cell infectivity as described in US20180066285A1.

d. Lentiviral Vectors

In some embodiments, the viral vector is a lentiviral vector. Lentiviruses are complex retroviruses, which, in addition to the common retroviral genes gag, pol, and env, contain other genes with regulatory or structural function. Information on lentiviral vectors is available, for example, in Naldini et al., Science 272(5259):263-267, 1996; Zufferey et al., Nat Biotechnol 15(9):871-875, 1997; Blomer et al., J Virol. 71(9):6641-6649, 1997; U.S. Pat. Nos. 6,013,516 and 5,994,136, each of which is incorporated herein by reference in its entirety. Some examples of lentivirus include the Human Immunodeficiency Viruses: HIV-1, HIV-2 and the Simian Immunodeficiency Virus: SIV. Lentiviral vectors have been generated by attenuating the HIV virulence genes, for example, the genes env, vif, vpr, vpu and nef are deleted to make the vector biologically safe. The lentivirus employed can also be replication and/or integration defective.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, which is incorporated herein by reference in its entirety. Those of skill in the art can target the recombinant virus by linkage of the envelope protein with an antibody or a particular ligand for targeting to a receptor of a particular cell type. For example, a target-specific vector can be generated by inserting a nucleic acid segment (including a regulatory region) of interest into the viral vector, along with another gene that encodes a ligand for a receptor on a specific target cell type.

Lentiviral vectors are known in the art, see Naldini et al., (1996 and 1998); Zufferey et al., (1997); Dull et al., 1998, U.S. Pat. Nos. 6,013,516; and 5,994,136 all incorporated herein by reference. In general, these vectors are plasmid-based or virus-based, and are configured to carry the essential sequences for incorporating foreign nucleic acid, for selection and for transfer of the nucleic acid into a host cell. In some cases, a lentiviral vector is introduced into a cell concurrently with one or more lentiviral packaging plasmids, which may include, without limitation, pMD2.G, pRSV-rev, pMDLG-pRRE, and pRRL-GOI. Introduction of a lentiviral vector alone or in combination with lentiviral packaging plasmids into a cell may cause the lentiviral vector to be packaged into a lentiviral particle.

### 2. Methods of Producing Viral Vectors

In general, a viral vector is produced by introducing a viral DNA or RNA construct into a producer cell. In some cases, the producer cell does not express exogenous genes. In other cases, the producer cell is a “packaging cell” comprising one or more exogenous genes, e.g., genes encoding one or more gag, pol, or env polypeptides and/or one or more retroviral gag, pol, or env polypeptides. The retroviral packaging cell may comprise a gene encoding a viral polypeptide, e.g., VSV-g, that aids entry into target cells. In some cases, the packaging cell comprises genes encoding one or more lentiviral proteins, e.g., gag, pol, env, vpr, vpu, vpx, vif, tat, rev, or nef. In some cases, the packaging cell comprises genes encoding adenovirus proteins such as E1 A or E1 B or other adenoviral proteins. For example, proteins supplied by packaging cells may be retrovirus-derived proteins such as gag, pol, and env; lentivirus-derived proteins such as gag, pol, env, vpr, vpu, vpx, vif, tat, rev, and nef; and adenovirus-derived proteins such as E1 A and E1 B. In many examples, the packaging cells supply proteins derived from a virus that differs from the virus from which the viral vector is derived. Methods of producing recombinant viruses from packaging cells and their uses are well established; see, e.g., U.S. Pat. Nos. 5,834,256; 6,910,434; 5,591,624; 5,817,491; 7,070,994; and 6,995,009.

Packaging cell lines include but are not limited to any easily-transfectable cell line. Packaging cell lines can be based on 293T cells, NIH3T3, COS or HeLa cell lines. Packaging cells are often used to package virus vector plasmids deficient in at least one gene encoding a protein required for virus packaging. Any cells that can supply a protein or polypeptide lacking from the proteins encoded by such viral vectors or plasmids may be used as packaging cells. Examples of packaging cell lines include but are not limited to: Platinum-E (Plat-E), Platinum-A (Plat-A), BOSC 23 (ATCC CRL 11554) and Bing (ATCC CRL 11270). Morita et al. (2000) Gene Therapy 7(12): 1063-1066; Onishi et al. (1996) Experimental Hematology, 24:324-329; U.S. Pat. No. 6,995,009. Commercial packaging lines are also useful, e.g., Ampho-Pak 293 cell line, Eco-Pak 2-293 cell line, RetroPack PT67 cell line, and Retro-X Universal Packaging System (all available from Clontech).

### 3. Plasmids

Virus vector plasmids (or constructs) include: pMXs, pMxs-IB, pMXs-puro, pMXs-neo (pMXs- IB is a vector carrying the blasticidin-resistant gene instead of the puromycin-resistant gene of pMXs-puro) Kimatura et al. (2003) Experimental Hematology 31 : 1007-1014; MFG Riviere et al. (1995) Proc. Natl. Acad. Sci., 92:6733-6737; pBabePuro; Morgenstern et al. (1990) Nucleic Acids Research 18:3587-3596; LL-CG, CL-CG, CS-CG, CLG Miyoshi et al. (1998) J. Vir. 72:8150-8157 and the like as the retrovirus system, and pAdexl Kanegae et al. (1995) Nucleic Acids Research 23 :3816-3821 and the like as the adenovirus system. In exemplary embodiments, the retroviral construct comprises blasticidin (e.g., pMXs-IB), puromycin (e.g., pMXs-puro, pBabePuro), or neomycin (e.g., pMXs-neo). Morgenstern et al. (1990) Nucleic Acids Research 18:3587-3596.

In some embodiments, the viral vector or plasmid comprises a transposon or a transposable element comprising a polynucleotide encoding a reprogramming factor. Delivery of polynucleotides via DNA transposons, such as piggyBac and Sleeping Beauty, offers advantages in ease of use, ability to delivery larger cargo, speed to clinic, and cost of production. The piggyBac DNA transposon, in particular, offers potential advantages in giving long-term, high-level and stable expression of polynucleotides, and in being significantly less mutagenic, being non-oncogenic and being fully reversible.

4. Direct Translation from Introduced RNA

When the one or more transgenes are expressed transiently in the selected cells, the gene(s) of interest can be introduced as an RNA molecule, which is translated to protein within the cell's cytoplasm. For example, the protein of interest can be translated from introduced RNA molecules that have the open reading frame (ORF) for the polypeptide flanked by a 5′ untranslated region (UTR) containing a translational initiation signal (e.g., a strong Kozak translational initiation signal) and a 3′ untranslated region terminating with an oligo(dT) sequence for templated addition of a polyA tail. Such RNA molecules do not have the promoter sequences employed in most expression vectors and expression cassettes. The RNA molecules can be introduced into the selected cells by a variety of techniques, including electroporation or by endocytosis of the RNA complexed with a cationic vehicle. See, e.g., Warren et al., Cell Stem Cell 7: 618-30 (2010), incorporated herein by reference in its entirety.

Protein translation can persist for several days, especially when the RNA molecules are stabilized by incorporation of modified ribonucleotides. For example, incorporation of 5-methylcytidine (5mC) for cytidine and/or pseudouridine (psi) for uridine can improve the half-life of the introduced RNA in vivo, and lead to increased protein translation. If high levels of expression are desired, or expression for more than a few days is desired, the RNA can be introduced repeatedly into the selected cells. The RNA encoding the protein can also include a 5′ cap, a nuclear localization signal, or a combination thereof.

Such RNA molecules can be made, for example, by in vitro transcription of a template for the polynucleotide of interest using a ribonucleoside blend that includes a 3′-O-Me-m7G(5′)ppp(5′)G ARCA cap analog, adenosine triphosphate and guanosine triphosphate, 5-methylcytidine triphosphate and pseudouridine triphosphate. The RNA molecules can also be treated with phosphatase to reduce cytotoxicity.

A microRNA can be expressed from an expression cassette or expression vector that has been introduced into a cell or a cell population. Alternatively, the microRNA can be introduced directly into cells, for example, in a delivery vehicle such as a liposome, microvesicle, or exosome. A single RNA can include both a protein-coding sequence and the microRNA.

### B. Non-Nucleic Acid Vectors

In certain embodiments, the vector comprises lipid particles as described in Kanasty R, Delivery materials for siRNA therapeutics Nat Mater. 12(11):967-77 (2013), which is hereby incorporated by reference. In some embodiments, the lipid-based vector is a lipid nanoparticle, which is a lipid particle between about 1 and about 100 nanometers in size.

In some embodiments, the lipid-based vector is a lipid or liposome. Liposomes are artificial spherical vesicles comprising a lipid bilayer.

In some embodiments, the lipid-based vector is a small nucleic acid-lipid particle (SNALP). SNALPs comprise small (less than 200 nm in diameter) lipid-based nanoparticles that encapsulate a nucleic acid. In some embodiments, the SNALP is useful for delivery of an RNA molecule such as siRNA. In some embodiments, SNALP formulations deliver nucleic acids to a particular tissue in a subject, such as the heart.

In some embodiments, the one or more polynucleotides are delivered via polymeric vectors. In some embodiments, the polymeric vector is a polymer or polymerosome. Polymers encompass any long repeating chain of monomers and include, for example, linear polymers, branched polymers, dendrimers, and polysaccharides. Linear polymers comprise a single line of monomers, whereas branched polymers include side chains of monomers. Dendrimers are also branched molecules, which are arranged symmetrically around the core of the molecule. Polysaccharides are polymeric carbohydrate molecules, and are made up of long monosaccharide units linked together. Polymersomes are artificial vesicles made up of synthetic amphiphilic copolymers that form a vesicle membrane, and may have a hollow or aqueous core within the vesicle membrane.

Various polymer-based systems can be adapted as a vehicle for administering DNA or RNA encoding the one or more reprogramming factors. Exemplary polymeric materials include poly(D,L-lactic acid-co-glycolic acid) (PLGA), poly(caprolactone) (PCL), ethylene vinyl acetate polymer (EVA), poly(lactic acid) (PLA), poly(L-lactic acid) (PLLA), poly(glycolic acid) (PGA), poly(L-lactic acid-co-glycolic acid) (PLLGA), poly(D,L-lactide) (PDLA), poly(L-lactide) (PLLA), PLGA-b-poly(ethylene glycol)-PLGA (PLGA-bPEG-PLGA), PLLA-bPEG-PLLA, PLGA-PEG-maleimide (PLGA-PEG-mal), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) (polyacrylic acids), and copolymers and mixtures thereof, polydioxanone and its copolymers, polyhydroxyalkanoates, polypropylene fumarate), polyoxymethylene, poloxamers, poly(ortho)esters, poly(butyric acid), poly(valeric acid), poly(lactide-co-caprolactone), trimethylene carbonate, polyvinylpyrrolidone, polyorthoesters, polyphosphazenes, Poly([beta]-amino esters (PBAE), and polyphosphoesters, and blends and/or block copolymers of two or more such polymers. Polymer-based systems may also include Cyclodextrin polymer (CDP)-based nanoparticles such as, for example, CDP-admantane (AD)-PEG conjugates and CDP-AD-PEG-transferrin conjugates.

In some embodiments, the lipid-based vector comprises a lipid encapsulation system. The lipid encapsulation system can be designed to drive the desired tissue distribution and cellular entry properties, as well as to provide the requisite circulation time and biodegrading character. The lipid encapsulation may involve reverse micelles and/or further comprise polymeric matrices, for example as described in U.S. Pat. No. 8,193,334, which is hereby incorporated by reference. In some embodiments, the particle includes a lipophilic delivery compound to enhance delivery of the particle to tissues, including in a preferential manner. Such compounds are disclosed in US 2013/0158021, which is hereby incorporated by reference in its entirety. Such compounds may generally include lipophilic groups and conjugated amino acids or peptides, including linear or cyclic peptides, and including isomers thereof. In some embodiments, the lipid encapsulation comprises one or more of a phospholipid, cholesterol, polyethylene glycol (PEG)-lipid, and a lipophilic compound.

The particles, whether lipid or polymeric or both, may include additional components useful for enhancing the properties for in vivo nucleic acid delivery (including compounds disclosed in U.S. Pat. No. 8,450,298 and US 2012/0251560, which are each hereby incorporated by reference). The delivery vehicle may accumulate preferentially in certain tissues thereby providing a tissue targeting effect, but in some embodiments, the delivery vehicle further comprises at least one cell-targeting or tissue-targeting ligand. Functionalized particles, including exemplary targeting ligands, are disclosed in US 2010/0303723 and 2012/0156135, which are hereby incorporated by reference in their entireties.

A delivery vehicle can be designed to drive the desired tissue distribution and cellular entry properties of the delivery systems disclosed herein, as well as to provide the requisite circulation time and biodegrading character. For example, lipid particles can employ amino lipids as disclosed in US 2011/0009641, which is hereby incorporated by reference.

The lipid or polymeric particles may have a size (e.g., an average size) in the range of about 50 nm to about 5 μm. In some embodiments, the particles are in the range of about 10 nm to about 100 μm, or about 20 nm to about 50 μm, or about 50 nm to about 5μm, or about 70 nm to about 500 nm, or about 70 nm to about 200 nm, or about 50 nm to about 100 nm. Particles may be selected so as to avoid rapid clearance by the immune system. Particles may be spherical, or non-spherical in certain embodiments.

### C. Promoters and Enhancers

In some embodiments, a nucleic acid encoding a reprogramming factor can be operably linked to a promoter and/or enhancer to facilitate expression of the reprogramming factor. Depending on the host/vector system utilized, any of a number of suitable transcription and translation control elements, including constitutive and inducible promoters, transcription enhancer elements, transcription terminators, etc. may be used in the expression vector (e.g., Bitter et al. (1987) Methods in Enzymology, 153 :516-544).

Separate promoters and/or enhancers can be employed for each of the polynucleotides. In some embodiments, the same promoter and/or enhancer is used for two or more polynucleotides in a single open reading frame. Vectors employing this configuration of genetic elements are termed “polycistronic.” An example of a polycistronic vector comprises an enhancer and a promoter operatively linked to a single open-reading frame comprising two or more polynucleotides linked by 2A region(s), whereby expression of the open-reading frame result in multiple polypeptides being generated co-translationally. The 2A region is believed to mediate generation of multiple polypeptide sequences through codon skipping; however, the present disclosure relates also to polycistronic vectors that employ post-translational cleavage to generate polypeptides for two or more genes of interest from the same polynucleotide. Illustrative 2A sequences, vectors, and associated methods are provided in US20040265955A1, which is incorporated herein by reference. Other polycistronic vectors of the disclosure employ internal promoter(s), splicing, reinitiation, internal ribosome entry site(s) (IRES), proteolytic cleavable site(s) (e.g. fusagen) and fusion of genes.

Non-limiting examples of suitable eukaryotic promoters (promoters functional in a eukaryotic cell) include CMV, CMV immediate early, HSV thymidine kinase, early and late SV40, long terminal repeats (LTRs) from retrovirus, and mouse metallothionein-I. In some embodiments, promoters that are capable of conferring cardiac-specific expression will be used. Non-limiting examples of suitable cardiac-specific promoters include desmin (Des), alpha-myosin heavy chain (a-MHC), myosin light chain 2 (MLC-2), cardiac troponin T (cTnT) and cardiac troponin C (cTnC). Non-limiting examples of suitable neuron specific promoters include synapsin I (SYN), calcium/calmodulin-dependent protein kinase II, tubulin alpha I, neuron-specific enolase and platelet-derived growth factor beta chain promoters and hybrid promoters by fusing cytomegalovirus enhancer (E) to those neuron-specific promoters.

Examples of suitable promoters for driving expression reprogramming factors include, but are not limited to, retroviral long terminal repeat (LTR) elements; constitutive promoters such as CMV, HSV1-TK, SV40, EF-1a, β-actin, phosphoglycerol kinase (PGK); inducible promoters, such as those containing Tet-operator elements; cardiac-specific promoters, such as desmin (Des), alpha-myosin heavy chain (a-MHC), myosin light chain 2 (MLC-2), cardiac troponin T (cTnT) and cardiac troponin C (cTnC); neural-specific promoters, such as nestin, neuronal nuclei (NeuN), microtubule-associate protein 2 (MAP2), beta III tubulin, neuron-specific enolase (NSE), oligodendrocyte lineage (Olig1/2), and glial fibrillary acidic protein (GFAP); and pancreatic-specific promoters, such as Pax4, Nkx2.2, Ngn3, insulin, glucagon, and somatostatin.

In some embodiments, a polynucleotide is operably linked to a cell type-specific transcriptional regulator element (TRE), where TREs include promoters and enhancers. Suitable TREs include, but are not limited to, TREs derived from the following genes: myosin light chain-2, α-myosin heavy chain, AE3, cardiac troponin C, and cardiac actin. Franz et al. (1997) Cardiovasc. Res. 35:560-566; Robbins et al. (1995) Ann. N. Y. Acad. Sci. 752:492-505; Linn et al. (1995) Circ. Res. 76:584-591; Parmacek et al. (1994) Cell. Biol. 14: 1870-1885; Hunter et al. (1993) Hypertension 22:608-617; and Sartorelli et al. (1992) Proc. Natl. Acad. Sci. USA 89:4047-4051.

The promoter can be one naturally associated with a gene or nucleic acid segment. Similarly, for RNAs (e.g., microRNAs), the promoter can be one naturally associated with a microRNA gene (e.g., an miRNA-302 gene). Such a naturally associated promoter can be referred to as the “natural promoter” and may be obtained by isolating the 5′ non-coding sequences located upstream of the coding segment and/or exon. Similarly, an enhancer may be one naturally associated with a nucleic acid sequence. However, the enhancer can be located either downstream or upstream of that sequence.

Alternatively, certain advantages will be gained by positioning the coding nucleic acid segment under the control of a recombinant or heterologous promoter, which refers to a promoter that is not normally associated with a nucleic acid in its natural environment. A recombinant or heterologous enhancer refers also to an enhancer not normally associated with a nucleic acid sequence in its natural environment. Such promoters or enhancers can include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference).

The promoters employed may be constitutive, inducible, developmentally-specific, tissue-specific, and/or useful under the appropriate conditions to direct high level expression of the nucleic acid segment. For example, the promoter can be a constitutive promoter such as, a CMV promoter, a CMV cytomegalovirus immediate early promoter, a CAG promoter, an EF-1α promoter, a HSV1-TK promoter, an SV40 promoter, a β-actin promoter, a PGK promoter, or a combination thereof. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. In some embodiments, the promoter comprises a CMV early enhancer element, chicken beta-actin promoter and an SV-40 intron. In some embodiments, the promoter comprises a CMV early enhancer element, chicken beta-actin promoter and a CMV intron. Other examples of promoters that can be employed include a human EFla elongation factor promoter, a CMV cytomegalovirus immediate early promoter, a CAG chicken albumin promoter, a viral promoter associated with any of the viral vectors described herein, or a promoter that is homologous to any of the promoters described herein (e.g., from another species). Examples of prokaryotic promoters that can be used include, but are not limited to, SP6, T7, T5, tac, bla, trp, gal, lac, or maltose promoters.

In some embodiments, an internal ribosome entry sites (IRES) element can be used to create multigene, or polycistronic, messages. IRES elements are able to bypass the ribosome scanning model of 5′-methylated Cap dependent translation and begin translation at internal sites (Pelletier and Sonenberg, Nature 334(6180):320-325 (1988)). IRES elements from two members of the picornavirus family (polio and encephalomyocarditis) have been described (Pelletier and Sonenberg, Nature 334(6180):320-325 (1988)), as well an IRES from a mammalian message (Macejak & Samow, Nature 353:90-94 (1991)). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

### D. Vector Delivery to Cells

The viral vector may be introduced into a target cell (e.g., fibroblast) by any method known in the art, including but not limited to: a calcium phosphate method, a lipofection method (e.g., Feigner et al. (1987) Proc. Natl. Acad. Sci. 84:7413-7417), an electroporation method, microinjection, Fugene transfection, nucleofection and the like, and any method described herein.

Examples of procedures include, for example, those described by Stadtfeld and Hochedlinger, Nature Methods 6(5):329-330 (2009); Yusa et al., Nat. Methods 6:363-369 (2009); Woltjen, et al., Nature 458, 766-770 (9 Apr. 2009)). Such methods include, but are not limited to, direct delivery of DNA such as by ex vivo transfection (e.g., Wilson et al., Science, 244:1344-1346, 1989, Nabel & Baltimore, Nature 326:711-713, 1987), optionally with Fugene6 (Roche) or Lipofectamine (Invitrogen); by injection (e.g., U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (e.g., Harland and Weintraub, J. Cell Biol., 101:1094-1099, 1985; U.S. Pat. No. 5,789,215, incorporated herein by reference in its entirety); by electroporation (e.g., U.S. Pat. No. 5,384,253, incorporated herein by reference in its entirety, Tur-Kaspa et al., Mol. Cell Biol., 6:716-718, 1986; Potter et al., Proc. Nat'l Acad. Sci. USA, 81:7161-7165, 1984); by calcium phosphate precipitation (e.g., Graham & Van Der Eb, Virology, 52:456-467, 1973; Chen and Okayama, Mol. Cell Biol., 7(8):2745-2752, 1987; Rippe et al., Mol. Cell Biol., 10:689-695, 1990); by use of DEAE-dextran followed by polyethylene glycol (e.g., Gopal, Mol. Cell Biol., 5:1188-1190, 1985); by direct sonic loading (e.g., Fechheimer et al., Proc. Nat'l Acad. Sci. USA, 84:8463-8467, 1987); by liposome-mediated transfection (e.g., Nicolau & Sene, Biochim. Biophys. Acta, 721:185-190, 1982, Fraley et al., Proc. Nat'l Acad. Sci. USA, 76:3348-3352, 1979; Nicolau et al., Methods Enzymol., 149:157-176, 1987, Wong et al., Gene, 10:87-94, 1980, Kaneda et al., Science, 243:375-378, 1989, Kato et al., Biol. Chem., 266:3361-3364, 1991), receptor-mediated transfection (e.g., Wu and Wu, Biochemistry, 27:887-892, 1988; Wu and Wu, J. Biol. Chem., 262:4429-4432, 1987); by endocytosis of the RNA complexed with a cationic vehicle (Warren et al., Cell Stem Cell 7: 618-30 (2010)); and any combination of such methods. Each of the foregoing references is incorporated herein by reference in its entirety.

Various techniques may be employed for introducing nucleic acid molecules of the disclosure into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like. Other examples include: N-TER™ Nanoparticle Transfection System by Sigma-Aldrich, FectoFly™ transfection reagents for insect cells by Polyplus Transfection, Polyethylenimine “Max” by Polysciences, Inc., Unique, Non-Viral Transfection Tool by Cosmo Bio Co., Ltd., Lipofectamine™ LTX Transfection Reagent by Invitrogen, SatisFection™ Transfection Reagent by Stratagene, Lipofectamine™ Transfection Reagent by Invitrogen, FuGENE® HD Transfection Reagent by Roche Applied Science, GMP compliant in vivo-jetPEI™ transfection reagent by Polyplus Transfection, and Insect GeneJuice® Transfection Reagent by Novagen.

### IV. Therapeutic Compositions

Reprogramming with a microRNA and one or both of ASCL1 and MYOCD can be combined with other reprogramming strategies in some cases with improved results. In some embodiments, the target tissues or starting cells express or are induced to express the OCT4 polypeptide. Target tissues or starting cells can be treated or incubated, respectively, with a reprogramming composition that contains one or more WNT agonists, GSK3 inhibitors, TGF-beta inhibitors, epigenetic modifiers, adenylyl cyclase agonists, OCT4 expression activators, and any combination thereof. The composition can contain at least two of such agents, or at least three of such agents, or at least four of such agents, or at least five of such agents, or at least six of such agents. For example, the composition can include SB431542 (an ALK4/5/7 inhibitor), CHIR99021 (a GSK3 inhibitor), parnate (an LSD1/KDM1 inhibitor, also called tranylcypromine) and forskolin (an adenylyl cyclase activator).

In certain embodiments, the reprogramming is enhanced by the administration of one or more anti-inflammatory agents, e.g., an anti-inflammatory steroid or a nonsteroidal anti-inflammatory drug (NSAID).

Anti-inflammatory steroids for use in the invention include corticosteroids, and in particular those with glucocorticoid activity, e.g., dexamethasone and prednisone. Nonsteroidal anti-inflammatory drugs (NSAIDs) for use in the invention generally act by blocking the production of prostaglandins that cause inflammation and pain, cyclooxygenase-1 (COX-1) and/or cyclooxygenase-2 (COX-2). Traditional NSAIDs work by blocking both COX-1 and COX-2. The COX-2 selective inhibitors block only the COX-2 enzyme. In certain embodiment, the NSAID is a COX-2 selective inhibitor, e.g., celecoxib (Celebrex®), rofecoxib (Vioxx), and valdecoxib (B extra). In certain embodiments, the anti-inflammatory is an NSAID prostaglandin inhibitor, e.g., Piroxicam.

To prepare the composition, the vectors and/or the cells are generated, and the vectors or cells are purified as necessary or desired. The vectors, cells, and/or other agents can be suspended in a pharmaceutically acceptable carrier. If the composition contains only compounds, without cells, the composition can be lyophilized. These compounds and cells can be adjusted to an appropriate concentration, and optionally combined with other agents. The absolute weight of a given compound and/or other agent included in a unit dose can vary widely. The dose and the number of administrations can be optimized by those skilled in the art.

For example, about 10²-10¹⁰ vector genomes (vg) may be administered. In some embodiments, the dose be at least about 10² vg, about 10³ vg, about 10⁴ vg, about 10⁵ vg, about 10⁶ vg, about 10⁷ vg, about 10⁸ vg, about 10⁹ vg, about 10¹⁰ vg, or more vector genomes. In some embodiments, the dose be about 10² vg, about 10³ vg, about 10⁴ vg, about 10⁵ vg, about 10⁶ vg, about 10⁷ vg, about 10⁸ vg, about 10⁹ vg, about 10¹⁰ vg, or more vector genomes.

It will be appreciated that the amount of vectors and cells for use in treatment will vary not only with the particular carrier selected but also with the route of administration, the nature of the condition being treated and the age and condition of the patient. Ultimately, the attendant health care provider may determine proper dosage. A pharmaceutical composition may be formulated with the appropriate ratio of each compound in a single unit dosage form for administration with or without cells. Cells or vectors can be separately provided and either mixed with a liquid solution of the compound composition, or administered separately.

One or more suitable unit dosage forms containing the compounds and/or the reprogrammed cells can be administered by a variety of routes including parenteral (including subcutaneous, intravenous, intramuscular and intraperitoneal), intracranial, intraspinal, oral, rectal, dermal, transdermal, intrathoracic, intrapulmonary and intranasal (respiratory) routes.

The vectors of the invention may be prepared in many forms that include aqueous solutions, suspensions, tablets, hard or soft gelatin capsules, and liposomes and other slow-release formulations, such as shaped polymeric gels. Administration of vectors often involves parenteral or local administration in an aqueous solution.

Liquid pharmaceutical compositions may be in the form of, for example, aqueous or oily suspensions, solutions, emulsions, syrups or elixirs, dry powders for reconstitution with water or other suitable vehicles before use. Such liquid pharmaceutical compositions may contain conventional additives such as suspending agents, emulsifying agents, non-aqueous vehicles (which may include edible oils), or preservatives.

Vectors can be formulated for parenteral administration (e.g., by injection, for example, bolus injection or continuous infusion) and may be presented in unit dosage form in ampoules, prefilled syringes, small volume infusion containers or multi-dose containers with an added preservative. The pharmaceutical compositions can take the form of suspensions, solutions, or emulsions in oily or aqueous vehicles, and can contain formulatory agents such as suspending, stabilizing and/or dispersing agents. Suitable carriers include saline solution, phosphate buffered saline, and other materials commonly used in the art.

The compositions can also contain other ingredients such as agents useful for treatment of cardiac diseases, conditions and injuries, such as, for example, an anticoagulant (e.g., dalteparin (fragmin), danaparoid (orgaran), enoxaparin (lovenox), heparin, tinzaparin (innohep), and/or warfarin (coumadin)), an antiplatelet agent (e.g., aspirin, ticlopidine, clopidogrel, or dipyridamole), an angiotensin-converting enzyme inhibitor (e.g., Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), and/or Trandolapril (Mavik)), angiotensin II receptor blockers (e.g., Candesartan (Atacand), Eprosartan (Teveten), Irbesartan (Avapro), Losartan (Cozaar), Telmisartan (Micardis), and/or Valsartan (Diovan)), a beta blocker (e.g., Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), and/or Timolol (Blocadren)), Calcium Channel Blockers (e.g., Amlodipine (Norvasc, Lotrel), Bepridil (Vascor), Diltiazem (Cardizem, Tiazac), Felodipine (Plendil), Nifedipine (Adalat, Procardia), Nimodipine (Nimotop), Nisoldipine (Sular), Verapamil (Calan, Isoptin, Verelan), diuretics (e.g, Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol) and/or Spironolactone (Aldactone)), vasodilators (e.g., Isosorbide dinitrate (Isordil), Nesiritide (Natrecor), Hydralazine (Apresoline), Nitrates and/or Minoxidil), statins, nicotinic acid, gemfibrozil, clofibrate, Digoxin, Digitoxin, Lanoxin, or any combination thereof

Additional agents can also be included such as antibacterial agents, antimicrobial agents, anti-viral agents, biological response modifiers, growth factors; immune modulators, monoclonal antibodies and/or preservatives. The compositions of the invention may also be used in conjunction with other forms of therapy.

The viral vectors and non-viral vectors described herein can be administered to a subject to treat a disease or disorder. Such a composition may be in a single dose, in multiple doses, in a continuous or intermittent manner, depending, for example, upon the recipient's physiological condition, whether the purpose of the administration is in response to traumatic injury or for more sustained therapeutic purposes, and other factors known to skilled practitioners. The administration of the compounds and compositions of the invention may be essentially continuous over a preselected period of time or may be in a series of spaced doses. Both local and systemic administration is contemplated. In some embodiments, localized delivery of a viral or non-viral vector is achieved. In some embodiments, localized delivery of cells and/or vectors is used to generate a population of cells within the heart. In some embodiments, such a localized population operates as “pacemaker cells” for the heart.

### V. Reprogramming Methods

As described herein, target cells (e.g., non-cardiomyocyte cells) can be reprogrammed to the cardiac lineage (e.g., cardiomyocyte lineage) in vivo by administration of a viral or non-viral vector to target tissues or cells. In some embodiments, the target cells are fibroblast cells. In some embodiments, the target cells are cardiac fibroblast (CF) cells.

In some embodiments, a vector or vectors comprising polynucleotide(s) encoding one or more reprogramming factors is administered to the subject, for example, ASCL1, MYOCD, MEF2C, TBX5, BAF60C, ESRRG, GATA4, GATA6, HAND2, IRX4, ISLL, MEF2C, MESP1, MESP2, NKX2.5, SRF, TBX20, ZFPM2, miR-133, or any combination thereof. In some embodiments, the reprogramming factors are selected from the group of ASCL1, MYOCD, and a microRNA selected from Table 1, or any combination thereof. In specific embodiments, the reprogramming factors are ASCL1 and MYOCD (MyA) and a microRNA selected from Table 1. In specific embodiments, the reprogramming factors are ASCL1, MYOCD, MEF2C and TBX5 (MyAMT), and a microRNA selected from Table 1. In some embodiments, the reprogramming factors are GATA4, MEF2C, and TBX5 (GMT) and a microRNA selected from Table 1. In other specific embodiments, the reprogramming factors are MYOCD, MEF2C, and TBX5 (i.e., MyMT), and a microRNA selected from Table 1. In other specific embodiments, the reprogramming factors are GATA4, MEF2C, TBX5, and MYOCD (i.e., 4F), and a microRNA selected from Table 1. In other embodiments, the reprogramming factors are GATA4, MEF2C, and TBX5, ESRRG, MYOCD, ZFPM2, and MESP1 (i.e., 7F), and a microRNA selected from Table 1.

In some embodiments, the vector induces expression or represses expression of more marker genes for cardiomyocytes, e.g., TNNT2, ACTN2, ATP2A2, MYH6, RYR2, MYH7, ACTCL, MYBPC3, PIN, MB, LMOD2, MYL2, MY 13, COX6A2, ATP5AL, TTN, TNNI3, PDK4, MYCZ2, CACNALC, SCN5A, MYOCD, and NPPA.

### VI. Methods of Treatment

The vectors described herein can be employed in a method of treating a subject with a cardiac disease or condition. “Treating” or “treatment of a condition or subject in need thereof” refers to (1) taking steps to obtain beneficial or desired results, including clinical results such as the reduction of symptoms; (2) preventing the disease, for example, causing the clinical symptoms of the disease not to develop in a patient that may be predisposed to the disease, but does not yet experience or display symptoms of the disease; (3) inhibiting the disease, for example, arresting or reducing the development of the disease or its clinical symptoms; (4) relieving the disease, for example, causing regression of the disease or its clinical symptoms; or (5) delaying the disease. For purposes of this invention, beneficial or desired clinical results include, but are not limited to, generating an induced cardiomyocyte and/or promoting myocardial regeneration.

Subjects in need of treatment using the compositions, cells and methods of the present disclosure include, but are not limited to, individuals having a congenital heart defect, individuals suffering from a degenerative muscle disease, individuals suffering from a condition that results in ischemic heart tissue (e.g., individuals with coronary artery disease), and the like. In some examples, a method is useful to treat a degenerative muscle disease or condition (e.g., familial cardiomyopathy, dilated cardiomyopathy, hypertrophic cardiomyopathy, restrictive cardiomyopathy, or coronary artery disease with resultant ischemic cardiomyopathy). In some examples, a subject method is useful to treat individuals having a cardiac or cardiovascular disease or disorder, for example, cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism.

Subjects who are suitable for treatment using the compositions, cells and methods of the present disclosure include individuals (e.g., mammalian subjects, such as humans, non-human primates, domestic mammals, experimental non-human mammalian subjects such as mice, rats, etc.) having a cardiac condition including but limited to a condition that results in ischemic heart tissue (e.g., individuals with coronary artery disease) and the like.

In some examples, an individual suitable for treatment suffers from a cardiac or cardiovascular disease or condition, e.g., cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism. In some examples, individuals suitable for treatment with a subject method include individuals who have a degenerative muscle disease, e.g., familial cardiomyopathy, dilated cardiomyopathy, hypertrophic cardiomyopathy, restrictive cardiomyopathy, or coronary artery disease with resultant ischemic cardiomyopathy.

Subjects in need of treatment using the compositions, cells and methods of the present disclosure include, but are not limited to, individuals having a congenital heart defect, individuals suffering from a degenerative muscle disease, individuals suffering from a condition that results in ischemic heart tissue (e.g., individuals with coronary artery disease), and the like. In some examples, a method is useful to treat a degenerative muscle disease or condition (e.g., familial cardiomyopathy, dilated cardiomyopathy, hypertrophic cardiomyopathy, restrictive cardiomyopathy, or coronary artery disease with resultant ischemic cardiomyopathy). In some examples, a subject method is useful to treat individuals having a cardiac or cardiovascular disease or disorder, for example, cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism.

Subjects who are suitable for treatment using the compositions, cells and methods of the present disclosure include individuals (e.g., mammalian subjects, such as humans, non-human primates, experimental non-human mammalian subjects such as mice, rats, etc.) having a cardiac condition including, but limited to, a condition that results in ischemic heart tissue (e.g., individuals with coronary artery disease) and the like. In some examples, an individual suitable for treatment suffers from a cardiac or cardiovascular disease or condition, e.g., cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism. In some examples, individuals suitable for treatment with a subject method include individuals who have a degenerative muscle disease, e.g., familial cardiomyopathy, dilated cardiomyopathy, hypertrophic cardiomyopathy, restrictive cardiomyopathy, or coronary artery disease with resultant ischemic cardiomyopathy.

Examples of diseases and conditions that can be treated using the reprogrammed cells and/or compositions (containing any of the compounds described herein with or without reprogrammed cells) include any cardiac pathology or cardiac dysfunction. Diseases and conditions that can be treated include those that occur as a consequence of genetic defect, physical injury, environmental insult or conditioning, bad health, obesity and other disease risk.

Ischemic cardiomyopathy is a chronic disorder caused by coronary artery disease (a disease in which there is atherosclerotic narrowing or occlusion of the coronary arteries on the surface of the heart). Coronary artery disease often leads to episodes of cardiac ischemia, in which the heart muscle is not supplied with enough oxygen-rich blood.

Non-ischemic cardiomyopathy is generally classified into three groups based primarily on clinical and pathological characteristics: dilated cardiomyopathy, hypertrophic cardiomyopathy and restrictive and infiltrative cardiomyopathy.

In another embodiment, the cardiac pathology is a genetic disease such as Duchenne muscular dystrophy and Emery Dreiffuss dilated cardiomyopathy.

For example, the cardiac pathology can be selected from the group consisting of congestive heart failure, myocardial infarction, cardiac ischemia, myocarditis and arrhythmia. In some embodiments, the subject is diabetic. In some embodiments, the subject is non-diabetic. In some embodiments, the subject suffers from diabetic cardiomyopathy.

For therapy, recombinant viruses, non-viral vectors, and/or pharmaceutical compositions can be administered locally or systemically. A reprogrammed population of cells can be introduced by injection, catheter, implantable device, or the like. A population of recombinant viruses or reprogrammed cells can be administered in any physiologically acceptable excipient or carrier that does not adversely affect the cells. For example, the recombinant viruses, non-viral vectors, and/or pharmaceutical compositions can be administered intravenously or through an intracardiac route (e.g., epicardially or intramyocardially). Methods of administering the recombinant viruses, non-viral vectors, cardiomyocytes and pharmaceutical compositions (e.g., compositions comprising vectors) of the disclosure to subjects, particularly human subjects include injection, implantation, or infusion of the pharmaceutical compositions (e.g., compositions comprising viral vectors) or cells into target sites in the subjects. Injection may include direct muscle injection and infusion may include intravascular infusion. The vectors or pharmaceutical compositions can be inserted into a delivery device which facilitates introduction by injection or implantation of the pharmaceutical compositions or cells into the subjects. Such delivery devices include tubes, e.g., catheters. The tubes can additionally include a needle, e.g., a syringe, through which the cells of the invention can be introduced into the subject at a desired location. In some embodiments, the pharmaceutical compositions or cells are delivered by microneedle patch as described in, for example, Tang et al. Cardiac cell—integrated microneedle patch for treating myocardial infarction. Science Advances 28 Nov. 2018: Vol. 4, no. 11, eaat9365.

In some aspects, a viral vector of the present disclosure can be used to treat a subject in need thereof. In some embodiments, the recombinant viruses can be administered to the subject in need thereof, where administration into the subject of the recombinant viruses, treats a cardiovascular disease in the subject.

Recombinant viruses may be administered locally or systemically. Recombinant viruses may be engineered to target specific cell types by selecting an appropriate capsid protein or by pseudotyping the virus with a protein from another virus type. To determine the suitability of various therapeutic administration regimens and dosages of viral particle compositions, the recombinant viruses can first be tested in a suitable animal model. At one level, recombinant viruses are assessed for their ability to infect target cells in vivo. Recombinant viruses can also be assessed to ascertain whether they migrate to target tissues, whether they induce an immune response in the host, or to determine an appropriate number, or dosage, of recombinant viruses to be administered. It may be desirable or undesirable for the recombinant viruses to generate an immune response, depending on the disease to be treated. Generally, if repeated administration of a viral particle is required, it will be advantageous if the viral particle is not immunogenic. For testing purposes, viral particle compositions can be administered to immunodeficient animals (such as nude mice, or animals rendered immunodeficient chemically or by irradiation). Target tissues or cells can be harvested after a period of infection and assessed to determine if the tissues or cells have been infected and if the desired phenotype (e.g. induced cardiomyocyte) has been induced in the target tissue or cells.

Recombinant viruses can be administered by various routes, including without limitation direct injection into the heart or cardiac catheterization. Alternatively, the recombinant viruses can be administered systemically such as by intravenous infusion. When direct injection is used, it may be performed either by open-heart surgery or by minimally invasive surgery. In some cases, the recombinant viruses are delivered to the pericardial space by injection or infusion. Injected or infused recombinant viruses can be traced by a variety of methods. For example, recombinant viruses labeled with or expressing a detectable label (such as green fluorescent protein, or beta-galactosidase) can readily be detected. The recombinant viruses may be engineered to cause the target cell to express a marker protein, such as a surface-expressed protein or a fluorescent protein. Alternatively, the infection of target cells with recombinant viruses can be detected by their expression of a cell marker that is not expressed by the animal employed for testing (for example, a human-specific antigen when injecting cells into an experimental animal). The presence and phenotype of the target cells can be assessed by fluorescence microscopy (e.g., for green fluorescent protein, or beta-galactosidase), by immunohistochemistry (e.g., using an antibody against a human antigen), by ELISA (using an antibody against a human antigen), or by RT-PCR analysis using primers and hybridization conditions that cause amplification to be specific for RNA indicative of a cardiac phenotype.

### VIII. Kits

A variety of kits are described herein that include any of the compositions, compounds and/or agents described herein. The kit can include any of the compounds described herein, either mixed together or individually packaged, and in dry or hydrated form. The compounds and/or agents described herein can be packaged separately into discrete vials, bottles or other containers. Alternatively, any of the compounds and/or agents described herein can be packaged together as a single composition, or as two or more compositions that can be used together or separately.

The vector can be provided within any of the kits in the form of a delivery device. Alternatively a delivery device can be separately included in the kit(s), and the instructions can describe how to assemble the delivery device prior to administration to a subject.

The kits can provide other factors such as any of the supplementary factors or drugs described herein for the compositions in the preceding section or other parts of the application.

The following non-limiting Examples illustrate some of the experimental work involved in developing the invention.

### EXAMPLES

### Example 1

This example employed an adeno-associated virus (AAV) vector that expresses two transgenes, myocardin (MYOCD) and ASCL1 (collectively termed “MyA”). The MyA reprogramming cocktail promotes direct reprogramming of cardiac fibroblasts into induced cardiomyocytes (iCMs) in vivo and in vitro (data not shown). The myocardin transgene used in this vector, termed MyΔ3 (SEQ ID NO: 16), has an internal deletion that reduces the size of the gene without appreciably impacting gene function (data not shown).

We designed a microRNA-based system to repress expression of MyA in non-target cells (cardiomyocytes) while expressing MyA in target cells (cardiac fibroblasts). The test vector was engineered to include microRNA binding site(s) for each of various selected microRNAs in the 3′ UTR of the polynucleotide encoding a MyΔ3-2A-ASCL1 transgene. The microRNA binding site(s) were intended to repress expression of MyA in cells that express the selected microRNA. Some experiments described below used a green-fluorescent protein (GFP) reporter system in place of MyA.

miR-1 Binding Sites

Two miR-1 binding sites were tested: a perfect complement to miR-1 (miR1_4) and a native miR-1 binding site in the 3′ UTR or human MYOCD (MymiR1_4). For each binding site, a mismatch negative control was generated in which the seed sequence (underline) was mutated (miR1_4mut, MymiR1_4mut):

miRl-1/1-2
(SEQ ID NO: 141)
3′-UAUGUAUGAAGAAAUGUAAGGU-5′
miR1_4
(SEQ ID NO: 142)
5′-AUACAUACUUCUUUACAUUCCA-3′
miR1_4mut
(SEQ ID NO: 143)
5′-AUACAUACUUCUUUACAgagCA-3′
MymiR1_4
(SEQ ID NO: 144)
5′-ACGAUGUCAUUUAACAUUCCG-3′
MymiR1_4mut
(SEQ ID NO: 145)
5′-ACGAUGUCAUUUAACAgagCG-3′

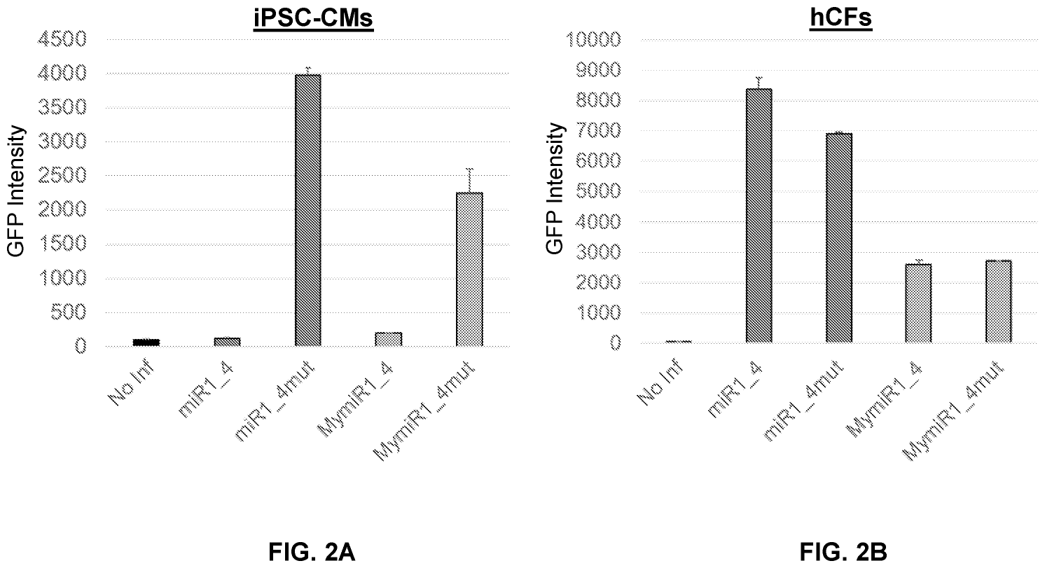

In each vector, the binding site to be tested was inserted in tandem four times into the 3′ UTR of an AAV CAG-GFP vector. The vectors were packaged into AAV and used to infect iPSC-CMs. iPSC-CMs are pluripotent stem cells induced to form cardiomyocytes, which are used in the experiment because maintaining primary cardiomyocytes in culture is impractical. GFP-miR1_4 and GFP-MymiR1_4 were both repressed in iPSC-CMs relative to control constructs GFP-miR1_4mut and GFP-MymiR1_4mut (FIG. 2A). Expression of the GFP reporter was still observed in human cardiac fibroblast (hCF) cells (FIG. 2B). The miR1_4 and GFP-MymiR1_4 constructs expressed the GFP reporter at about the same level as their miR-1 mismatch controls. Thus, miR-1 microRNA binding sites selectively repress transgene expression in CM cells compared to CF cells.

The miR1_4 and miR1_4mut sequences were inserted into the 3′ UTR of the AAV My ^{m}A cassette to assess the impact of the binding sites on cardiomyocyte reprogramming by the combination of myocardin and ASCL1 (MyA). Reprogramming efficiency was determined by measuring expression of seven genes associated with cardiomyocyte phenotype. The data in Table 5 demonstrate that at day 7 after transduction of hCF cells with the vectors, the MyA vector with miR-1 microRNA binding sites induced expression of CM phenotype.

TABLE 5

| | MYOCD | ASCL1 | CASQ2 | ACTC1 | NPPA | PLN | TNNT2 |
|---|---|---|---|---|---|---|---|
| no vector | 1.0 | 1.0 | 1.1 | 1.0 | 1.4 | 1.0 | 1.00 |
| My^{Δ3} A | 1911.1 | 21794.4 | 885.4 | 545.7 | 35551.0 | 158.5 | 665.3 |
| My^{Δ3} A_miR1_4mut | 2221.0 | 25468.2 | 805.3 | 491.3 | 25610.8 | 202.1 | 688.4 |
| My^{Δ3} A_miR1_4 | 1353.8 | 13703.1 | 82.8 | 81.7 | 1954.6 | 45.7 | 163.4 |

The test vector My^{Δ3}A_miR1_4 showed reprogramming activity at 5-10 fold lower potency than My^{Δ3}A_miR1_4mut or the parental My^{Δ3}A cassette.

Next, vectors with one, two, or three miR-1 binding sites were compared to the vector with four miR-1 binding sites. As shown in Table 6, the vector with only one miR-1 binding site, My^{Δ3}A_miR1_1, exhibited greater potency than My^{Δ3}A_miR1_4, but remained less potent than the parental My^{Δ3}A vector.

TABLE 6

| | ASCL1 | MYOCD | NPPA | TNNT2 |
|---|---|---|---|---|
| no vector | 0.7 | 0.9 | 1.4 | 1.2 |
| | 1.5 | 1.1 | 1.3 | 0.9 |
| My^{Δ3} A | 8295.8 | 2383.1 | 17204.8 | 625.5 |
| | 9270.1 | 2352.9 | 19162.1 | 598.4 |
| My^{Δ3} A_miR1_ | 10747.8 | 3419.4 | 17009.0 | 594.1 |
| 4mut | 10243.2 | 2694.1 | 13668.2 | 583.5 |
| My^{Δ3} A_miR1_1 | 7523.6 | 2419.9 | 1113.9 | 217.0 |
| | 5419.9 | 1789.6 | 1320.8 | 163.8 |
| My^{Δ3} A_miR1_2 | 6882.7 | 1932.6 | 1292.5 | 170.5 |
| | 5126.6 | 1433.8 | 915.1 | 105.4 |
| My^{Δ3} A_miR1_3 | 4292.3 | 1628.4 | 554.4 | 139.2 |
| | 4796.4 | 1467.3 | 1010.8 | 127.5 |
| My^{Δ3} A_miR1_4 | 5258.8 | 1644.2 | 602.4 | 119.4 |
| | 5285.4 | 1498.5 | 690.0 | 114.2 |

Constructs were generated containing two (miR1_1mis2) or three (miR1_1mis3) mismatches outside of the seed region in a single miR-1 binding site in the 3′ UTR of My^{Δ3}A to decrease targeting of these miR-1 binding sites without abolishing it.

| (SEQ ID NO: 141) | |
|---|---|
| miR-1 | UAUGUAUGAAGAAAUGUAAGGU |
| (SEQ ID NO: 142) | |
| miR1_1 | AUACAUACUUCUUUACAUUCCA |
| (SEQ ID NO: 146) | |
| miR1_1mis2 | AUACAUAGUUGUUUACAUUCCA |
| (SEQ ID NO: 147) | |
| miR1_1mis3 | AUACAUAGUUGUCUACAUUCCA |

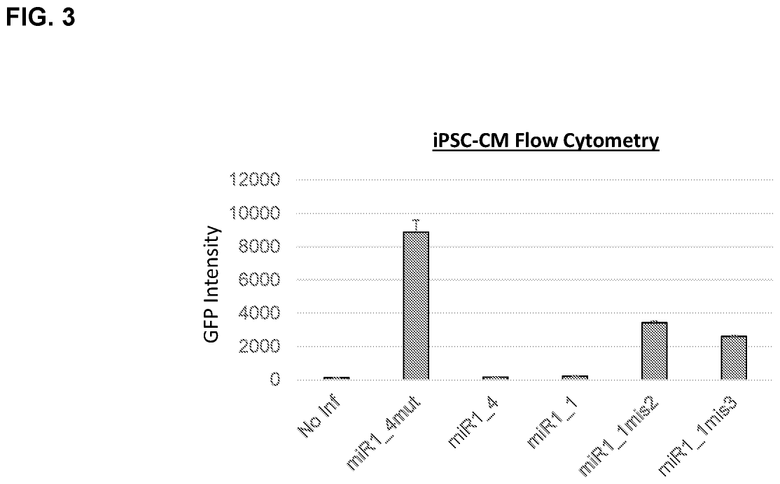

These constructs increased reprogramming potency relative to MyΔ3A_miR1_1 (Table 7), while repressing transgene expression in cardiomyocytes less well (FIG. 3).

TABLE 6

| | ASCL1 | MYOCD | CASQ2 | NPPA | TNNT2 |
|---|---|---|---|---|---|
| no vector | 1.1 | 1.0 | 1.2 | 2.6 | 1.1 |
| My^{Δ3} A | 2675.0 | 1026.1 | 1053.5 | 23166.2 | 870.2 |
| My^{Δ3} A_ | 2479.7 | 1116.7 | 742.7 | 15176.0 | 656.4 |
| miR1_4mut | | | | | |
| My^{Δ3} A_ | 1773.7 | 805.7 | 130.7 | 1823.3 | 213.6 |
| miR1_1 | | | | | |
| My^{Δ3} A_ | 1853.8 | 776.0 | 434.0 | 6980.6 | 422.4 |
| miR1_1mis2 | | | | | |
| My^{Δ3} A_ | 2623.7 | 1007.5 | 515.3 | 9044.4 | 476.8 |
| miR1_1mis3 | | | | | |

miR-208a/b Binding Sites

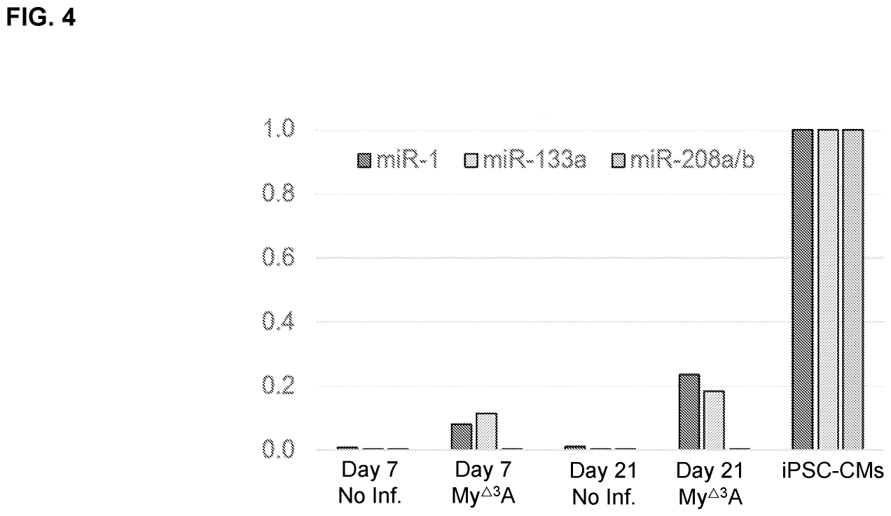

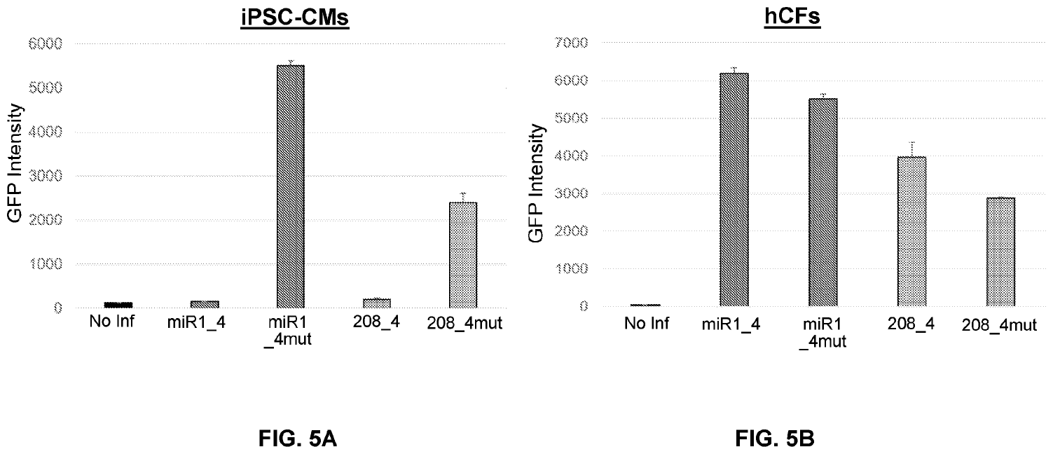

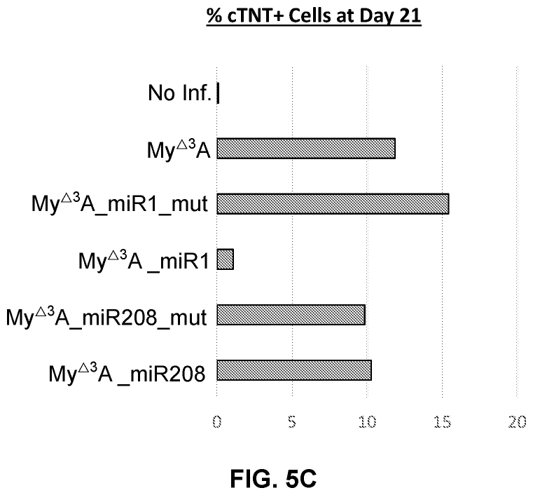

We observed that during the cardiomyocyte reprogramming process induced by My^{Δ3}A , increased expression of microRNAs miR-1 and miR-133 precede increased expression of miR-208a/b (FIG. 4). The microRNA miR-208a/b was detected in iPSC-CMs but not in hCFs undergoing reprogramming for up to three weeks. An AAV CAG-GFP vector having a 4× tandem array of perfectly complementary miR-208b binding sites (208_4), as well as a mismatch negative control vector with the seed site mutated (208_4mut), was generated and used to infect human iPSC-CMs. GFP-208_4 repressed transgene expression as well as GFP-miR1_4 (FIG. 5A). In hCFs, both GFP-208_4 and GFP-208_4mut expressed the transgene at a lower level relative to GFP-miR1_4 and GFP-miR1_4mut (FIG. 5B). My^{Δ3}A_208_4 and My^{Δ3}A_208_4mut were both able to drive robust reprogramming, as assessed by expression of RNA markers 21 days after transduction of hCF cells (Table 7 and FIG. 5C). My^{Δ3}A_208_4 and My^{Δ3}A_208_4mut exhibited similar reprogramming potency.

TABLE 7

| | NPPA | TNNT2 |
|---|---|---|
| no vector | 1.1 | 1.0 |
| | 1.9 | 1.0 |
| My^{Δ3} A | 31900.1 | 108.4 |
| | 56894.4 | 169.6 |
| My^{Δ3} A_miR1_mut | 59336.0 | 167.5 |
| | 43705.0 | 154.1 |
| My^{Δ3} A_miR1 | 1169.5 | 15.1 |
| | 1456.2 | 20.8 |
| My^{Δ3} A_miR208_mut | 34120.0 | 114.0 |
| | 31381.7 | 112.5 |
| My^{Δ3} A_miR208 | 25148.6 | 109.1 |
| | 28183.4 | 105.5 |

### In Vivo Testing in Mice

For in vivo testing, My^{Δ3}A_208_4 was packaged into either an AAV5 capsid having wild-type capsid sequence or an AAV5z capsid, a variant with greater cardiac fibroblast infectivity (data not shown), and tested in mice using the LAD-ligation mouse model of myocardial infarction (MI), with injection of the AAV vector performed epicardially at the time of injury. Vector design and dosing are summarized in Table 8.

TABLE 8

| | Groups | Dose | Mice# |
|---|---|---|---|
| 1 | AAV5: GFP | 1.2 × 10¹¹ GC | 7 |
| 2 | AAV5z: GFP | 1.2 × 10¹¹ GC | 8 |
| 3 | AAV5: My^{Δ3} A | 1.2 × 10¹¹ GC | 15 |
| 4 | AAV5: My^{Δ3} A_208_4 | 1.2 × 10¹¹ GC | 15 |
| 5 | AAV5z: My^{Δ3} A_208_4 | 1.2 × 10¹¹ GC | 15 |

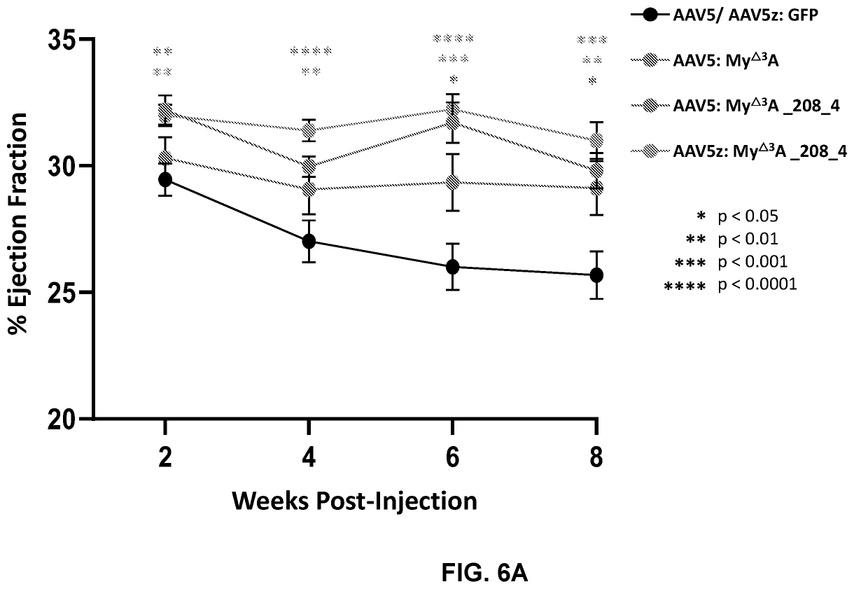

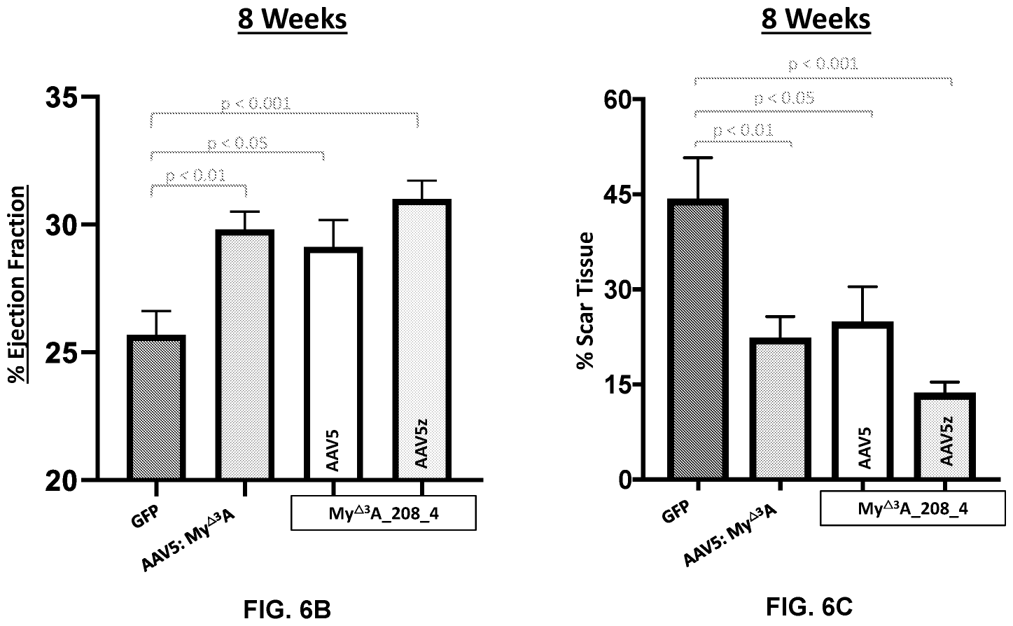

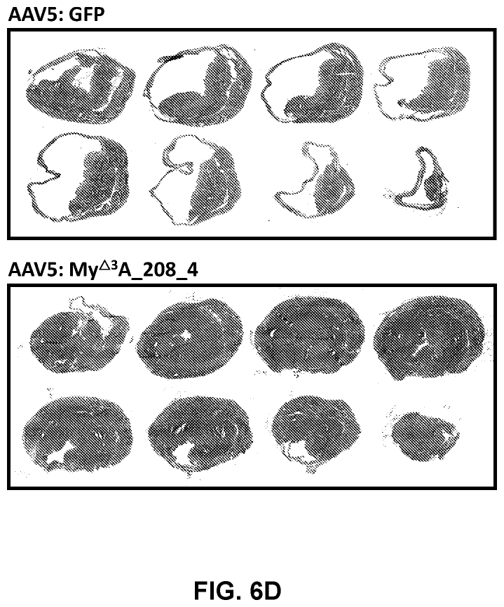

Efficacy was assessed by echocardiography at 2, 4, 6, and 8 weeks post-injection. All three test articles significantly preserved cardiac function (ejection fraction) compared to the GFP-encoding control (FIG. 6A). After 8 weeks, the three test articles significantly preserved cardiac function (ejection fraction), with no statistically significant difference in reprogramming efficiency between MyA and MyA+miR-208 binding site vectors (FIG. 6B). AAV5:My^{Δ3}A and AAV5:My^{Δ3}A_208_4 were comparably efficacious in MI when delivered in the same viral capsid, AAV5.

Terminal scar analysis with trichrome staining and quantification of the percentage of fibrotic tissue per cardiac cross-section showed that all three treatment groups exhibited significant reduction in scar area (FIG. 6C), as shown in representative micrographs (FIG. 6D).

### In Vivo Testing in Pigs

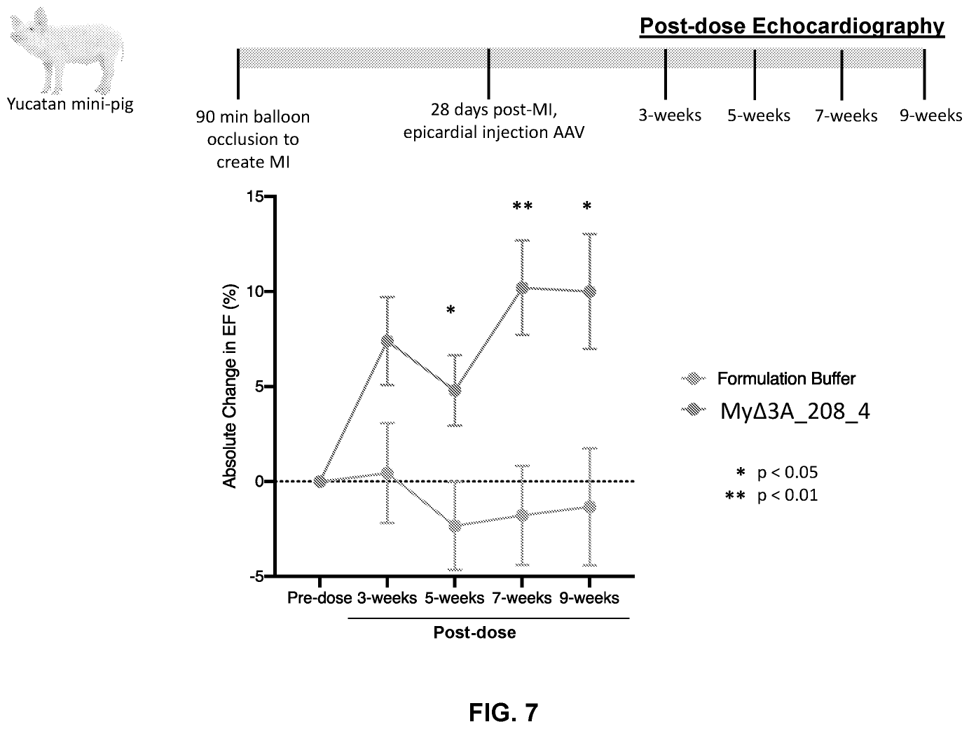

The vector was further tested in a pig myocardial infarction (MI) model. My^{Δ3}A_208_4 was packaged into AAV5z capsid and injected epicardially into the border zone of pig hearts 28 days after a 90-minute balloon occlusion to introduce ischemic injury. In parallel, infarcted pigs were also injected with formulation buffer, so that each group consisted of ten animals. Echocardiography was performed 3-weeks, 5-weeks, 7-weeks and 9-weeks post-injection. Pigs that received My^{Δ3}A_208_4 demonstrated significant improvement in ejection fraction relative to control 5-weeks, 7-weeks and 9-weeks post-injection (FIG. 7). Thus, treatment with the cardiomyocyte-detargeted reprogramming cocktail resulted in an average 10% improvement in cardiac performance above pre-dose baseline.

### Improved miR-1 and miR-208a/b Binding Sites

Variants of the miR-208 binding site that disrupt a predicted RNA hairpin in the microRNA binding site, by introducing substitutions in the two 5′ nucleotide positions, were tested, with results shown in Table 9.

| (SEQ ID NO: 148) | |
|---|---|
| miR-208b | UGUUUGGAAAACAAGCAGAAUA |
| (SEQ ID NO: 149) | |
| miR208 | ACAAACCUUUUGUUCGUCUUAU |
| (SEQ ID NO: 150) | |
| miR208_melt | UGAAACCUUUUGUUCGUCUUAU |
| (SEQ ID NO: 151) | |
| miR208_meltmut | UGAAACCUUUUGUUCGUGAGAU |

TABLE 9

| | NPPA | TNNT2 |
|---|---|---|
| no vector | 2.0 | 0.9 |
| | 1.2 | 1.2 |
| My^{Δ3} A | 17101.6 | 1254.6 |
| | 18991.2 | 1458.7 |
| My^{Δ3} A_208_4mut | 7866.3 | 759.6 |
| | 7967.1 | 805.7 |
| My^{Δ3} A_208_4 | 8299.2 | 821.6 |
| | 7008.7 | 805.3 |
| My^{Δ3} A_208_4meltmut | 14532.9 | 1249.7 |
| | 17627.5 | 1247.2 |
| My^{Δ3} A_208_4melt | 6688.9 | 724.4 |
| | 9748.2 | 797.4 |

Further sequence variants outside of the seed region were tested to identify microRNA binding sites that would represses expression in iPSC-CMs while permitting transgene expression in hCFs and resulting in high quality iCMs after 3 weeks of reprogramming (see Table 10).

TABLE 10

| | # | | iPSC- | | | |
|---|---|---|---|---|---|---|
| Construct | Sites | Mismatches | CMs | hCFs | iCMs | Sequence |
| miR1_4 | 4 | 0 | | +++ | −/+ | ATACATACTTCTTTACATTCCA |
| | | | | | | (SEQ ID NO: 152) |
| miR1_4mut | 4 | 3 SEED | +++ | +++ | +++ | ATACATACTTCTTTACAgagCA |
| | | | | | | (SEQ ID NO: 153) |
| MymiR1_4 | 4 | native | −/+ | +/++ | n.d. | ACGATGTCATTTAACATTCCG |
| | | | | | | (SEQ ID NO: 154) |
| My miR1_4mut | 4 | 3 SEED | + | +/++ | n.d. | ACGATGTCATTTAACAgagCG |
| | | | | | | (SEQ ID NO: 155) |
| miR1_1 | 1 | 0 | | +++ | −/+ | ATACATACTTCTTTACATTCCA |
| | | | | | | (SEQ ID NO: 156) |
| miR1_2 | 2 | 0 | n.d. | n.d. | −/+ | ATACATACTTCTTTACATTCCA |
| | | | | | | (SEQ ID NO: 157) |
| miR1_3 | 3 | 0 | n.d. | n.d. | −/+ | ATACATACTTCTTTACATTCCA |
| | | | | | | (SEQ ID NO: 158) |
| miR1_1mis2 | 1 | 2 | +/++ | nd. | +/++ | ATACATAGTTGTTTACATTCCA |
| | | | | | | (SEQ ID NO: 159) |
| miR1_1mis3 | 1 | 3 | +/++ | nd. | +/++ | ATACATAGTTGTCTACATTCCA |
| | | | | | | (SEQ ID NO: 160) |
| miR1_1TB4 | 1 | native | +/++ | n.d. | +/++ | AATATGCACTGTACATTCCA |
| | | | | | | (SEQ ID NO: 161) |
| 208_4 | 4 | 0 | | | | ACAAACCTTTTGTTCGTCTTAT |
| | | | | | | (SEQ ID NO: 135) |
| 208_4mut | 4 | 2 SEED | | | | ACAAACCTTTTGTTCGTCgaAT |
| | | | | | | (SEQ ID NO: 162) |
| 208_2 | 2 | 0 | | | | ACAAACCTTTTGTTCGTCTTAT |
| | | | | | | (SEQ ID NO: 135) |
| 208_4melt | 4 | 2 | | | | TGAAACCTTTTGTTCGTCTTAT |
| | | | | | | (SEQ ID NO: 137) |
| 208_4mut3 | 4 | 3 SEED | +++ | +++ | +++ | TGAAACCTTTTGTTCGTgagAT |
| | | | | | | (SEQ ID NO: 163) |
| 208_moMEL1 | 4 | 3 | +/++ | | | TGAAACCTTTTGTTCGTCTTAT |
| | | | | | | (SEQ ID NO: 164) |
| 208_moMEL2 | 4 | 4 | | | | TGATAGCTTTTGTTCGTCTTAT |
| | | | | | | (SEQ ID NO: 165) |
| 208MED13 | 4 | native | −/+ | +++ | +++ | AAAATATATGTAATCGTCTTAA |
| | | | | | | (SEQ ID NO: 136) |
| 208_chim | 4 | 4 | +/++ | | | ATACATACTTCGTTCGTCTTAT |
| | | | | | | (SEQ ID NO: 166) |
| | | ideal result | − | +++ | +++ | |

iPSC-CM = pluripotent stem cells previously differentiated into cardiomyocytes.
hCF = human cardiac fibroblasts.
iCM = human cardiac fibroblasts treated with AAV vector encoding My A for three weeks-i.e., in process of differentiating into cardiomyocytes.

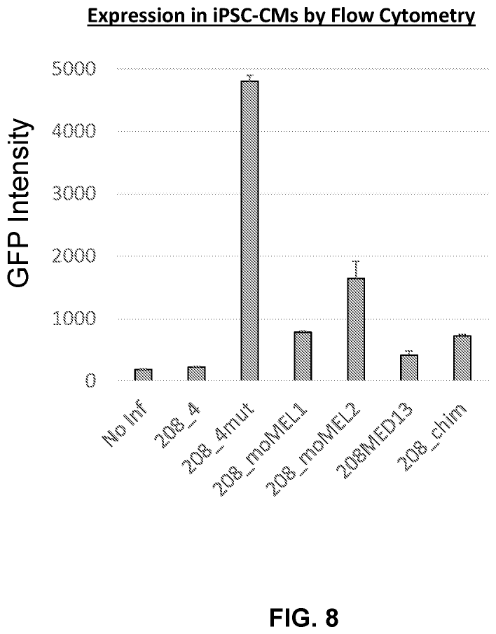

Several of these sets miR-208 binding sites were tested their ability to repress transgene expression in iPSC-CMs (FIG. 8) and maintain or increase in vitro reprogramming potency compared to the parental vector (Table 9). 208MED13, an insertion based on the native miR-208 targeting site in the MED13 transcript performed well, boosting reprogramming potency while retaining iPSC-CM repression.

### Example 2

This example employed an adeno-associated virus (AAV) vector that expresses two transgenes, myocardin (MYOCD) with internal deletion and ASCL1 (My^{Δ3}A), plus a polynucleotide encoding the microRNA-133 (i.e. a MyA133 three-factor combination). This sequence encoding miR-133 is inserted into the intron between the CAG promoter and the MYOCD (My^{Δ3}) coding sequence.

As shown in Table 11, addition of miR133 to My^{Δ3}A increased reprogramming, as assessed by RNA expression of cardiomyocytes on day 21 after transduction with AAV vector at MOI of 640k. Both 208_4 and 208MED13 microRNA binding sites preserve the reprogramming effectiveness of the vector, with 208MED13 showing better results than 208_4.

TABLE 11

| | ASCL1 | MYOCD | NPPA | TNNT2 | CASQ2 | MYH6 | TNNC1 |
|---|---|---|---|---|---|---|---|
| no vector | 1.0 | 1.0 | 1.0 | 1.0 | 1.0 | 1.0 | 1.0 |
| My^{Δ3} A | 3263.2 | 480.8 | 93021.7 | 103.0 | 2807.8 | 284.2 | 166.7 |
| My^{Δ3} A_208_4 | 2260.6 | 360.3 | 45307.8 | 65.3 | 2184.6 | 153.8 | 96.0 |
| My^{Δ3} A_208MED13 | 2274.2 | 328.8 | 65595.4 | 82.2 | 2608.5 | 229.2 | 122.5 |
| [133]-My^{Δ3} A | 2745.4 | 404.8 | 140836.9 | 116.9 | 4337.4 | 650.1 | 296.6 |
| [133]-My^{Δ3} A_208_4 | 2093.7 | 335.9 | 68343.5 | 87.8 | 3029.2 | 389.9 | 246.8 |
| [133]-My^{Δ3} A_208MED13 | 2452.8 | 391.3 | 107163.2 | 115.1 | 4008.7 | 584.9 | 324.6 |

Results were confirmed using two different viral doses in an independently derived hCF cell line, shown in Table 12.

TABLE 12

| | ASCL1 | MYOCD | NPPA | TNNT2 | ACTC1 | CASQ2 | MYH6 | TNNC1 |
|---|---|---|---|---|---|---|---|---|
| NO INF. | 1.6 | 1.0 | 1.7 | 1.0 | 2.2 | 1.0 | 1.0 | 1.0 |

MOI 160K

| | | | | | | | | |
|---|---|---|---|---|---|---|---|---|
| My^{Δ3} A | 11990.4 | 5005.5 | 129562.0 | 850.6 | 87606.8 | 2123.9 | 390.7 | 403.8 |
| My^{Δ3} A_208MED13 | 11601.3 | 4384.3 | 104344.4 | 668.1 | 73309.3 | 2059.4 | 473.7 | 370.6 |
| [133]-My^{Δ3} A | 10749.2 | 4416.2 | 242293.2 | 862.8 | 94985.9 | 4116.0 | 1324.9 | 853.2 |
| [133]-My^{Δ3} A_208MED13 | 10272.2 | 4233.5 | 197659.7 | 653.3 | 80234.7 | 4196.3 | 1401.9 | 738.9 |

MOI 640K

| | | | | | | | | |
|---|---|---|---|---|---|---|---|---|
| My^{Δ3} A | 14774.5 | 6651.8 | 148557.6 | 953.3 | 100463.3 | 2298.6 | 494.2 | 512.0 |
| My^{Δ3} A_208MED13 | 16895.8 | 6011.2 | 125222.6 | 838.6 | 85719.8 | 2217.3 | 487.5 | 465.3 |
| [133]-My^{Δ3} A | 14441.0 | 5441.3 | 261500.1 | 1026.5 | 111517.6 | 5341.2 | 2362.0 | 1211.0 |
| [133]-My^{Δ3} A_208MED13 | 13431.2 | 5241.6 | 233383.7 | 766.9 | 98889.0 | 5019.7 | 2125.1 | 1014.1 |

The addition of miR-133 boosted the expression of many cardiac markers ˜two-fold after three weeks of in vitro reprogramming. With the addition of miR-133a, there was no difference in potency between the My^{Δ3}A and My^{Δ3}A_208MED13 cassettes, indicating miR-133a addition did not result in premature repression of the cassette.

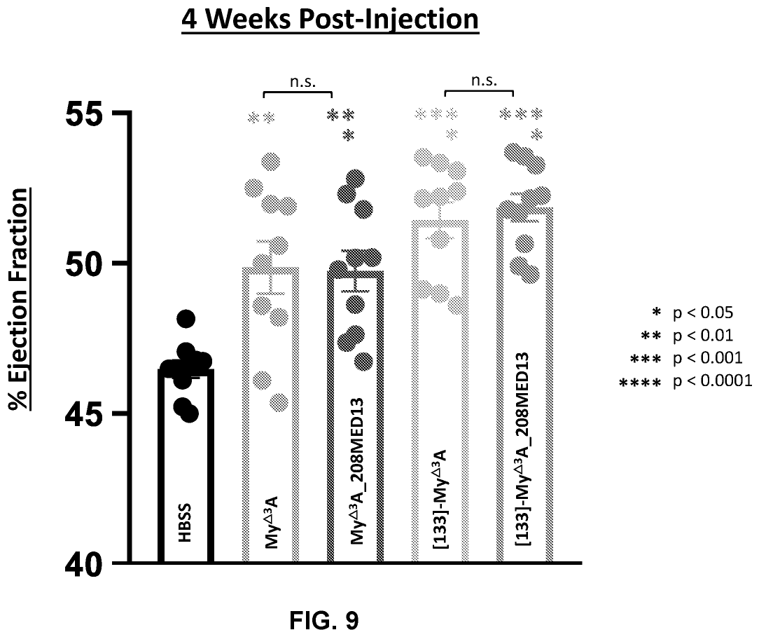

The My^{Δ3}A and My^{Δ3}A+133 vectors with the 208MED13 miR-208 microRNA binding site were tested in a rat LAD-ligation model of ischemic injury. Viral injections were performed two weeks post-ligation and treatment animals were randomized to ensure equivalent baseline ejection fraction between treatment groups. The data shown in FIG. 9 demonstrate that all test articles preserve ejection fraction compared to vehicle control (HBSS) (n=10 rats/group). No significant difference was discernible between My^{Δ3}A and My^{Δ3}A_208MED13 independent of miR-133a inclusion into the cassette.

### Materials and Methods

Isolation of primary adult human fibroblasts. For isolation of adult human cardiac fibroblasts (AHCFs), adult human left ventricles were minced into small pieces and digested in cardiac fibroblast digestion medium (10 μg/ml Liberase TH, 10 μg/ml Liberase™, 1 unit/ml DNase I, 0.01% Polaxomer) for 1 h in 37° C. After digestion, the cells were filtered through a 70 μM strainer into a 50 mL falcon tube. Cells were pelleted by spinning down for 5 min at 1200×g and placed in fibroblast growth medium. The medium was replaced every two days. Four days later, AHCFs were frozen or re-plated for viral transduction.

Cellular reprogramming. For in vitro cardiac reprogramming, AHCFs were seeded into culture dishes or plates at a density of 5×10³/cm² in fibroblast growth medium (day −1). One day after plating cells (day 0), fibroblast growth medium was removed and virus medium was added. One day after viral transduction (day 1), virus medium was replaced by iCM medium that composed of 4 parts Dulbecco's Modified Eagle's Medium (DMEM) and 1 part Gibco® Media 199, 10% FBS, 1% nonessential amino acids, 1% penicillin/streptomycin, for every two days until day 4. On day 4, medium was changed to 75% iCM media and 25% RPMI and B27. On day 7, medium was changed to 50% iCM and 50% RPMI+B27. On day 11, medium was changed to 25% iCM and 75% RPMI and B27. On day 14, medium was changed to RPMI and B27 and FFV (long/ml rhFGF, 15ng/m1 rhFGF-10, and 5ng/ml rhVEGF) for every day until day 21.

Immunocytochemistry. For immunocytochemistry, cells were fixed in 4% paraformaldehyde for 20 min and permeabilized with 0.1% Triton-X100 at room temperature for 30 min. Cells were washed with PBS three times followed by blocking with 1% bovine serum albumin (BSA) for 1 h. Cells then were incubated with mouse monoclonal anti-cardiac Troponin T (cTnT) antibody (Thermo Scientific, MA5-12960) at 1:200 dilutions or mouse anti-α-actinin antibody (Sigma, A7811) at 1:200 dilutions in 1% BSA for 1 h. After washing with PBS three times, cells were then incubated with donkey anti-mouse Alexa Fluor 594 (Invitrogen, A21203) at 1:200 dilutions in 1% BSA for 1 h. Cells were then imaged and quantified using a cell imaging multi-mode reader, Cytation™ 5 (BioTek).

Quantification and statistical analysis. All data are presented as mean with standard error of the mean (SEM) and have n=2-3 per group. P values were calculated with either unpaired/two-way t test or one-way analysis of variance (ANOVA). Statistical analyses were performed using the GraphPad Prism® 7 software package (GraphPad Software™). A P value of <0.05 was considered significant in all cases after corrections were made for multiple pairwise comparisons.

MI surgery, epicardial injection of AAV and echocardiography. Mouse surgeries were performed on CHARLES RIVER® CD-1 IGS male mice of 9-10 weeks of age. Mice were anesthetized with 2.4% isoflurane/97.6% oxygen and placed in a supine position on a heating pad (37° C.). Animals were intubated with a 20-gauge intravenous catheter and ventilated with a mouse ventilator (MINIVENT™, Harvard Apparatus, Inc.). MI was induced by permanent ligation of the left anterior descending artery (LAD) with a 7-0 prolene suture. 20 ul of AAV (1.2E11 GC total) was injected into the myocardium through an insulin syringe with an incorporated 29 gauge needle (BD). Injection with a full dosage was carried out along the boundary between the infarct zone (IZ) and border zone (BZ) based on the blanched infarct area after coronary artery occlusion. After injection, the chest was closed with sutures. All surgical procedures were performed under aseptic conditions. Rat infarcts were generated as above, but viral dosing was performed two weeks following LAD ligation with three 30 ul injections for a total of 3E11 GC. Cardiac function was evaluated by two-dimensional transthoracic echocardiography on conscious mice using a VISUALSONICSTM VEVO® 3100 imaging system. Ejection fraction (EF), end systolic volume (ESV) and end diastolic volume (EDV) were used as indices of cardiac contractile function. All pig procedures and echocardiography were performed at Charles River, Mattawan. Male castrated Yucatan minipigs were balloon occluded for 90 minutes under observation, then open-chest epicardial injections were performed 28 days after occlusion. Test article or formulation buffer was delivered in ten injections of 500 ul each in the border zone, resulting in a total dose of E14 GC.

### Example 3

The mRNA and protein stability of various forms of detargeted constructs in the presence of endogenous miR-208 is assessed in human iPSC-derived cardiomyocytes (hiPSC-CMs). hiPSC-CMs are infected with AAV containing the following cassettes:

My^{Δ3}A_208

My^{Δ3}A_208mut

My^{Δ3}A-CMVInt-133_MED13

My^{Δ3}A-CMVInt-133_208

My^{Δ3}A-CMVInt_208

Cells are be harvested at 4 and 14 days post-transduction. RNA levels of the MyΔ3A transcript are measured by qRT-PCR; protein levels of ASCL1 are assessed by Western blot. Effective detargeting results in decreased protein levels compared to the construct containing the mutated miR-208 site.

The mRNA and protein stability of these same constructs are also assessed in response to varying levels of miR-208. HEK293 cells, which do not express endogenous miR-208, are infected with the constructs listed above. Two days later, they are transfected with varying levels of miR-208a or miR-208b. Cells are harvested at 4 days post-transfection. RNA levels of the My^{Δ3}A transcript are measured by qRT-PCR; protein levels of ASCL1 are assessed by Western blot. The relative dose-response of each detargeted construct are determined from these results.

Expression from various detargeted constructs will also be assessed in the porcine model, in vivo. The following seven cassettes are produced in AAV:

| | | | | miR-208 |
|---|---|---|---|---|
| No. | Intron | Transgene | miR-133 | binding site |
| 1 | SV40 | My^{Δ3} A | not present | Perfect match |
| 2 | CMV | My^{Δ3} A | not present | Perfect match |
| 3 | CMV | My^{Δ3} A | present | Perfect match |
| 4 | SV40 | My^{Δ3} A | not present | Med13 |
| 5 | CMV | My^{Δ3} A | not present | Med13 |
| 6 | CMV | My^{Δ3} A | present | Med13 |
| 7 | CMV | My^{Δ3} A | present | not present |

A pool of the seven constructs is delivered into the hearts of six healthy Yorkshire pigs by open-chest epicardial injection at three discrete sites. Each injection site is marked by suture of a glass bead for identification at necropsy. Animals are euthanized at 4 weeks (n=3) or 12 weeks (n=3) post-injection. Biopsy punches from each injection site is collected at necropsy for RNA and DNA analyses. RNA expression versus vector genomes delivered for each of the seven vectors at both timepoints is compared.

What is claimed is:

1. A vector, comprising a polynucleotide comprising a polynucleotide sequence encoding one or more transgenes and a microRNA binding site for a microRNA, wherein the microRNA binding site is operatively linked to the polynucleotide sequence encoding the one or more transgenes, and wherein the microRNA is expressed at a higher level in cardiomyocytes or cardiomyocyte progenitors compared to cardiac fibroblasts.

2. The vector of claim 1, wherein the microRNA binding site promotes specific repression of expression of the one or more transgenes in a cardiomyocyte or cardiomyocyte progenitor compared to a cardiac fibroblast.

3. The vector of claim 1 or claim 2, wherein the microRNA is expressed at a lower level in cardiac fibroblasts and/or is expressed at a lower level in cardiac fibroblasts treated with the cardiomyocytes reprogramming factor for about 7 days or less, compared to a level of expression of the microRNA in cardiomyocytes and/or a level of expression of the microRNA in cardiac fibroblasts treated with the cardiomyocytes reprogramming factor for more than about 7 days.

4. The vector of any one of claims 1 to 3, wherein the microRNA is miR-208.

5. The vector of any one of claims 1 to 3, wherein the microRNA is miR-1.

6. The vector of any one of claims 1 to 3, wherein the microRNA is miR-133.

7. The vector of claim 4, wherein the microRNA is miR-208a.

8. The vector of claim 4, wherein the microRNA is miR-208b.

9. The vector of claim 8, wherein the microRNA is miR-208b-3p.

10. The vector of claim 9, wherein the microRNA binding site shares >70% identity to ACAAACCTTTTGTTCGTCTTAT (SEQ ID NO: 135) and no mismatches in the underlined seed region comprising the sequence CGTCTTA.

11. The vector of claim 9, wherein the microRNA binding site is
(SEQ ID NO: 136)
AAAATATATGTAATCGTCTTAA.

12. The vector of claim 9, wherein the microRNA binding site is
(SEQ ID NO: 135)
ACAAACCTTTTGTTCGTCTTAT.

13. The vector of claim 9, wherein the microRNA binding site is
(SEQ ID NO: 137)
TGAAACCTTTTGTTCGTCTTAT.

14. The vector of any one of claims 1 to 13, wherein the polynucleotide comprises at least two microRNA binding sites for the microRNA.

15. The vector of claim 14, wherein the polynucleotide comprises at least four microRNA binding sites for the microRNA.

16. The vector of claim 14 or claim 15, wherein the polynucleotide comprises at most six microRNA binding sites for the microRNA.

17. The vector of claim 16, wherein the polynucleotide comprises four microRNA binding sites for the microRNA.

18. The vector of any one of claims 1 to 17, wherein the one or more transgenes comprises one or more cardiomyocyte reprogramming factors.

19. The vector of claim 18, wherein the one or more cardiomyocyte reprogramming factors comprises two or more of MYOCD, ASCL1, GATA4, MEF2C, TBXS, miR-133, and MESP1.

20. The vector of claim 18, wherein the one or more cardiomyocyte reprogramming factors comprises three or more of MYOCD, ASCL1, GATA4, MEF2C, TBXS, miR-133, and MESP1.

21. The vector of claim 18, wherein the one or more cardiomyocyte reprogramming factors comprise MYOCD and ASCL1.

22. The vector of claim 21, wherein the polynucleotide sequence encodes a MYOCD-2A-ASCL1 protein.

23. The vector of any one of claims 20 to 22, wherein the MYOCD comprises an internal deletion.

24. The vector of claim 23, wherein the polynucleotide comprises, in 5′ to 3′ order, a promoter, a sequence encoding MYOCD and ASCL1, the microRNA binding site, and a polyadenylation sequence.

25. The vector of any one of claims 18 to 24, wherein the polynucleotide comprises a sequence encoding miR-133.

26. The vector of claim 24 or claim 25, wherein the polynucleotide comprises a sequence at least 95% identical to SEQ ID NO: 138, SEQ ID NO: 139 or SEQ ID NO: 140.

27. The vector of any one of claims 1 to 26, wherein the vector is a viral vector.

28. The vector of claim 27, wherein the viral vector is an adeno-associated virus (AAV) vector.

29. A method for reprogramming cardiac fibroblasts into cardiomyocytes, comprising:
a) selecting a microRNA specifically expressed in induced cardiomyocytes by treating cardiac fibroblasts with an effective amount of a composition that induces reprogramming of cardiac fibroblasts to cardiomyocytes and measuring the expression of one or more microRNAs in the cardiac fibroblasts, wherein the selected microRNA is expressed in the cardiac fibroblasts only after a predetermined time;
b) generating a vector comprising a polynucleotide comprising one or more microRNA binding sites for the selected microRNA operatively linked to a polynucleotide encoding one or more cardiomyocyte reprogramming factors; and
c) contacting a cardiac fibroblast with an effective amount of the vector.

30. The method of claim 29, wherein the microRNA binding site represses expression of the one or more cardiomyocyte reprogramming factors in cardiomyocyte cells.

31. The method of claim 29 or claim 30, wherein the microRNA binding site represses expression of the one or more cardiomyocyte reprogramming factors in skeletal muscle cells.

32. The method of any one of claims 29 to 31, wherein the microRNA binding site represses expression of the one or more cardiomyocyte reprogramming factors in cardiomyocyte progenitor cells.

33. The method of any one of claims 29 to 32, wherein the microRNA is miR-208.

34. The method of any one of claims 29 to 32, wherein the microRNA is miR-1.

35. The method of any one of claims 29 to 32, wherein the microRNA is miR-133.

36. The method of claim 33, wherein the microRNA is miR-208a.

37. The method of claim 33, wherein the microRNA is miR-208b.

38. The method of claim 37, wherein the microRNA is miR-208b-3p.

39. The method of claim 38, wherein the microRNA binding site shares >70% identity to ACAAACCTTTTGTTCGTCTTAT (SEQ ID NO: 135) and no mismatches in the underlined seed region comprising the sequence CGTCTTA.

40. The method of claim 38, wherein the microRNA binding site is
(SEQ ID NO: 136)
AAAATATATGTAATCGTCTTAA.

41. The method of claim 38, wherein the microRNA binding site is
(SEQ ID NO: 135)
ACAAACCTTTTGTTCGTCTTAT.

42. The method of claim 38, wherein the microRNA binding site is
(SEQ ID NO: 137)
TGAAACCTTTTGTTCGTCTTAT.

43. The method of any one of claims 29 to 42, wherein the polynucleotide comprises at least two microRNA binding sites for the microRNA.

44. The method of claim 43, wherein the polynucleotide comprises at least four microRNA binding sites for the microRNA.

45. The method of claim 43, wherein the polynucleotide comprises at most six microRNA binding sites for the microRNA.

46. The method of claim 43, wherein the polynucleotide comprises four microRNA binding sites for the microRNA.

47. A method for reprogramming a cardiac fibroblast into a cardiomyocyte cell, comprises contacting the cardiac fibroblast with an effective amount of the vector of any one of claims 1 to 28.

48. The method of claim 47, wherein the method induces expression of at least one marker of cardiomyocyte phenotype in the cardiac fibroblast.

49. The method of claim 48, where at least one marker of cardiomyocyte phenotype is a messenger RNA level of ASCL1, MYOCD, CASQ2, NPPA, or TNNT2

50. A method of promoting formation of cardiomyocytes in a subject in need thereof, comprising administering the vector of any one of claims 1 to 28 to the subject.

51. A method of treating heart failure in a subject in need thereof, comprising administering the vector of any one of claims 1 to 28 to the subject.

52. A method of treating heart failure in a subject in need thereof, comprising administering to the subject an AAV vector comprising a polynucleotide comprising in 5′ to 3′ order, a promoter, a sequence encoding MYOCD and ASCL1, a microRNA binding site, and a polyadenylation sequence, wherein the microRNA binding site is a microRNA binding site for miR-1, miR-133, miR-208a, miR-208b, and/or miR-208b-3p.

53. The method of claim 52, wherein the microRNA binding site is a microRNA binding site for miR-1.

54. The method of claim 52, wherein the microRNA binding site is a microRNA binding site for miR-133.

55. The method of claim 52, wherein the microRNA binding site is a microRNA binding site for miR-208a.

56. The method of claim 52, wherein the microRNA binding site is a microRNA binding site for miR-208b.

57. The method of claim 52, wherein the microRNA binding site is a microRNA binding site for miR-208b-3p.

58. The method of any one of claims 52 to 57, wherein the polynucleotide comprises a sequence encoding miR-133.

59. The method of any one of claims 52 to 58, wherein the heart failure is due to myocardial infarction.

60. The method of any one of claims 52 to 59, wherein the heart failure is heart failure with reduced ejection fraction (HFrEF).

61. The method of any one of claims 52 to 60, wherein the method increases ejection fraction in the subject compared to the subject before administration.

62. The method of any one of claims 52 to 61, wherein the method increases ejection fraction in the subject compared to an untreated control subject.

63. The method of any one of claims 52 to 62, wherein the method increases ejection fraction in the subject to at least about 28%, 29%, 30%, 31%, or 32%.

64. The method of any one of claims 61 to 64, wherein ejection fraction is assessed after a predetermined time, optionally eight weeks after administration of the AAV vector.

65. The method of any one of claims 52 to 64, wherein the method decreases scar tissue formation in the subject compared to the subject before administration.

66. The method of any one of claims 52 to 65, wherein the method decreases scar tissue formation in the subject compared to an untreated control subject.

67. The method of any one of claim 65 or claim 66, wherein the method decreases scar tissue formation in the subject to at most about 10%, 11%, 12%, 13%, 14%, 15%, 16%, 17%, 18%, 19%, or 20%.

68. The method of any one of claims 65 to 67, wherein scar tissue formation is assessed after a predetermined time, optionally eight weeks after administration of the AAV vector.


SEQUENCE LISTING (166 sequences)

SEQ ID NO: 1 - protein, 236 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 2 - DNA, 711 bp, Homo sapiens
atggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctga

SEQ ID NO: 3 - protein, 986 aa, Homo sapiens
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 4 - DNA, 2961 bp, Homo sapiens
atgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccagggccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtag

SEQ ID NO: 5 - protein, 463 aa, Homo sapiens
MGRKKIQITRIMDERNRQVTFTKRKFGLMKKAYELSVLCDCEIALIIFNSTNKLFQYASTDMDKVLLKYTEYNEPHESRTNSDIVEALNKKENKGCESPDPDSSYALTPRTEEKYKKINEEFDNMIKSHKIPAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 6 - DNA, 1392 bp, Homo sapiens
atggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggaggcattgaacaagaaagaaaacaaaggctgtgaaagccccgatcccgactcctcttatgcactcaccccacgcactgaagaaaaatacaaaaaaattaatgaagaatttgataatatgatcaagagtcataaaattcctgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacatga

SEQ ID NO: 7 - protein, 518 aa, Homo sapiens
MADADEGFGLAHTPLEPDAKDLPCDSKPESALGAPSKSPSSPQAAFTQQGMEGIKVFLHERELWLKFHEVGTEMIITKAGRRMFPSYKVKVTGLNPKTKYILLMDIVPADDHRYKFADNKWSVTGKAEPAMPGRLYVHPDSPATGAHWMRQLVSFQKLKLTNNHLDPFGHIILNSMHKYQPRLHIVKADENNGFGSKNTAFCTHVFPETAFIAVTSYQNHKITQLKIENNPFAKGFRGSDDMELHRMSRMQSKEYPVVPRSTVRQKVASNHSPFSSESRALSTSSNLGSQYQCENGVSGPSQDLLPPPNPYPLPQEHSQIYHCTKRKEEECSTTDHPYKKPYMETSPSEEDSFYRSSYPQQQGLGASYRTESAQRQACMYASSAPPSEPVPSLEDISCNTWPSMPSYSSCTVTTVQPMDRLPYQHFSAHFTSGPLVPRLAGMANHGSPQLGEGMFQHQTSVAHQPVVRQCGPQTGLQSPGTLQPPEFLYSHGVPRTLSPHQYHSVHGVGMVPEWSDNS

SEQ ID NO: 8 - DNA, 1557 bp, Homo sapiens
atggccgacgcagacgagggctttggcctggcgcacacgcctctggagcctgacgcaaaagacctgccctgcgattcgaaacccgagagcgcgctcggggcccccagcaagtccccgtcgtccccgcaggccgccttcacccagcagggcatggagggaatcaaagtgtttctccatgaaagagaactgtggctaaaattccacgaagtgggcacggaaatgatcataaccaaggctggaaggcggatgtttcccagttacaaagtgaaggtgacgggccttaatcccaaaacgaagtacattcttctcatggacattgtacctgccgacgatcacagatacaaattcgcagataataaatggtctgtgacgggcaaagctgagcccgccatgcctggccgcctgtacgtgcacccagactcccccgccaccggggcgcattggatgaggcagctcgtctccttccagaaactcaagctcaccaacaaccacctggacccatttgggcatattattctaaattccatgcacaaataccagcctagattacacatcgtgaaagcggatgaaaataatggatttggctcaaaaaatacagcgttctgcactcacgtctttcctgagactgcgtttatagcagtgacttcctaccagaaccacaagatcacgcaattaaagattgagaataatccctttgccaaaggatttcggggcagtgatgacatggagctgcacagaatgtcaagaatgcaaagtaaagaatatcccgtggtccccaggagcaccgtgaggcaaaaagtggcctccaaccacagtcctttcagcagcgagtctcgagctctctccacctcatccaatttggggtcccaataccagtgtgagaatggtgtttccggcccctcccaggacctcctgcctccacccaacccatacccactgccccaggagcatagccaaatttaccattgtaccaagaggaaagaggaagaatgttccaccacagaccatccctataagaagccctacatggagacatcacccagtgaagaagattccttctaccgctctagctatccacagcagcagggcctgggtgcctcctacaggacagagtcggcacagcggcaagcttgcatgtatgccagctctgcgccccccagcgagcctgtgcccagcctagaggacatcagctgcaacacgtggccaagcatgccttcctacagcagctgcaccgtcaccaccgtgcagcccatggacaggctaccctaccagcacttctccgctcacttcacctcggggcccctggtccctcggctggctggcatggccaaccatggctccccacagctgggagagggaatgttccagcaccagacctccgtggcccaccagcctgtggtcaggcagtgtgggcctcagactggcctgcagtcccctggcacccttcagccccctgagttcctctactctcatggcgtgccaaggactctatcccctcatcagtaccactctgtgcacggagttggcatggtgccagagtggagcgacaatagctaa

SEQ ID NO: 9 - protein, 20 aa, Homo sapiens
KRKEEECSTTDHPYKKPYME

SEQ ID NO: 10 - protein, 409 aa, Homo sapiens
GSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVP

SEQ ID NO: 11 - protein, 223 aa, Homo sapiens
EVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 12 - protein, 434 aa, Homo sapiens
GSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSS

SEQ ID NO: 13 - protein, 555 aa, Homo sapiens
GSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSE

SEQ ID NO: 14 - protein, 636 aa, Artificial Sequence
  Made in Lab - Engineered MYOCD protein
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 15 - protein, 661 aa, Artificial Sequence
  Made in Lab - Engineered MYOCD protein
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 16 - protein, 782 aa, Artificial Sequence
  Made in Lab - Engineered MYOCD protein
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 17 - protein, 116 aa, Homo sapiens
GSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLE

SEQ ID NO: 18 - protein, 111 aa, Homo sapiens
SASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQ

SEQ ID NO: 19 - protein, 54 aa, Homo sapiens
SSFKPGPLPPNLDDLKVSELRQQLRIRGLPVSGTKTALMDRLRPFQDCSGNPVP

SEQ ID NO: 20 - protein, 41 aa, Homo sapiens
LDGLDSEKDKMLVEKQKVINELTWKLQQEQRQVEELRMQLQ

SEQ ID NO: 21 - DNA, 57 bp, Porcine teschovirus-1
gccacgaacttctctctgttaaagcaagcaggagacgtggaagaaaaccccggtcct

SEQ ID NO: 22 - DNA, 57 bp, Porcine teschovirus-1
gctactaacttcagcctgctgaagcaggctggagacgtggaggagaaccctggacct

SEQ ID NO: 23 - protein, 19 aa, Porcine teschovirus-1
ATNFSLLKQAGDVEENPGP

SEQ ID NO: 24 - DNA, 54 bp, Thoseaasigna virus
gagggcagaggaagtctgctaacatgcggtgacgtcgaggagaatcctggacct

SEQ ID NO: 25 - protein, 18 aa, Thoseaasigna virus
EGRGSLLTCGDVEENPGP

SEQ ID NO: 26 - DNA, 60 bp, Equine rhinitis A virus
cagtgtactaattatgctctcttgaaattggctggagatgttgagagcaaccctggacct

SEQ ID NO: 27 - protein, 20 aa, Equine rhinitis A virus
QCTNYALLKLAGDCESNPGP

SEQ ID NO: 28 - DNA, 66 bp, Foot-and-mouth disease virus
gtgaaacagactttgaattttgaccttctcaagttggcgggagacgtggagtccaaccctggacct

SEQ ID NO: 29 - protein, 22 aa, Foot-and-mouth disease virus
VKQTLNFDLLKLAGDVESNPGP

SEQ ID NO: 30 - protein, 6 aa, Artificial Sequence
  Made in Lab - linker peptide
GGSGGS

SEQ ID NO: 31 - protein, 6 aa, Artificial Sequence
  Made in Lab - linker peptide
GSSGGS

SEQ ID NO: 32 - protein, 6 aa, Artificial Sequence
  Made in Lab - linker peptide
GGSGSS

SEQ ID NO: 33 - protein, 9 aa, Artificial Sequence
  Made in Lab - linker peptide
GGSGGSGGS

SEQ ID NO: 34 - protein, 12 aa, Artificial Sequence
  Made in Lab - linker peptide
GGSGGSGGSGGS

SEQ ID NO: 35 - DNA, 3726 bp, Artificial Sequence
  Made in Lab - synthesized MYOCD-2A-ASCL1 construct
atgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccaggcccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtgggccacgaacttctctctgttaaagcaagcaggagacgtggaagaaaaccccggtcctatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctga

SEQ ID NO: 36 - DNA, 3744 bp, Artificial Sequence
  Made in Lab - synthesized MYOCD-2A-MYF6 construct
atgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccaggcccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtgggccacgaacttctctctgttaaagcaagcaggagacgtggaagaaaaccccggtcctatgatgatggacctttttgaaactggctcctatttcttctacttggatggggaaaatgttactctgcagccattagaagtggcagaaggctctcctttgtatccagggagtgatggtaccttgtccccctgccaggaccaaatgcccccggaagcggggagcgacagcagcggagaggaacatgtcctggcgcccccgggcctgcagcctccacactgccccggccagtgtctgatctgggcttgcaagacctgcaagagaaaatctgcccccactgaccggcgaaaagccgccaccctgcgcgaaaggaggaggctaaagaaaatcaacgaggccttcgaggcactgaagcggcgaactgtggccaaccccaaccagaggctgcccaaggtggagattctgcggagcgccatcagctatattgagcggctgcaggacctgctgcaccggctggatcagcaggagaagatgcaggagctgggggtggaccccttcagctacagacccaaacaagaaaatcttgagggtgcggatttcctgcgcacctgcagctcccagtggccaagtgtttccgatcattccagggggctcgtgataacggctaaggaaggaggagcaagtattgattcgtcagcctcgagtagccttcgatgcctttcttccatcgtggacagtatttcctcggaggaacgcaaactcccctgcgtggaggaagtggtggagaagtaa

SEQ ID NO: 37 - DNA, 3114 bp, Artificial Sequence
  Made in Lab - synthesized MyDelta3-2A-ASCL1 construct
atgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccaggcccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagaggaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtgggccacgaacttctctctgttaaagcaagcaggagacgtggaagaaaaccccggtcctatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctga

SEQ ID NO: 38 - DNA, 3132 bp, Artificial Sequence
  Made in Lab - Synthesized MyDelta3-2A-MYF6 construct
atgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccaggcccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagaggaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtgggccacgaacttctctctgttaaagcaagcaggagacgtggaagaaaaccccggtcctatgatgatggacctttttgaaactggctcctatttcttctacttggatggggaaaatgttactctgcagccattagaagtggcagaaggctctcctttgtatccagggagtgatggtaccttgtccccctgccaggaccaaatgcccccggaagcggggagcgacagcagcggagaggaacatgtcctggcgcccccgggcctgcagcctccacactgccccggccagtgtctgatctgggcttgcaagacctgcaagagaaaatctgcccccactgaccggcgaaaagccgccaccctgcgcgaaaggaggaggctaaagaaaatcaacgaggccttcgaggcactgaagcggcgaactgtggccaaccccaaccagaggctgcccaaggtggagattctgcggagcgccatcagctatattgagcggctgcaggacctgctgcaccggctggatcagcaggagaagatgcaggagctgggggtggaccccttcagctacagacccaaacaagaaaatcttgagggtgcggatttcctgcgcacctgcagctcccagtggccaagtgtttccgatcattccagggggctcgtgataacggctaaggaaggaggagcaagtattgattcgtcagcctcgagtagccttcgatgcctttcttccatcgtggacagtatttcctcggaggaacgcaaactcccctgcgtggaggaagtggtggagaagtaa

SEQ ID NO: 39 - DNA, 3726 bp, Artificial Sequence
  Made in Lab - synthesized ASCL1-2A-MYOCD construct
atggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttcgccacgaacttctctctgttaaagcaagcaggagacgtggaagaaaaccccggtcctatgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccaggcccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtag

SEQ ID NO: 40 - DNA, 3744 bp, Artificial Sequence
  Made in Lab - synthesized MYF6-2A-MYOCD construct
atgatgatggacctttttgaaactggctcctatttcttctacttggatggggaaaatgttactctgcagccattagaagtggcagaaggctctcctttgtatccagggagtgatggtaccttgtccccctgccaggaccaaatgcccccggaagcggggagcgacagcagcggagaggaacatgtcctggcgcccccgggcctgcagcctccacactgccccggccagtgtctgatctgggcttgcaagacctgcaagagaaaatctgcccccactgaccggcgaaaagccgccaccctgcgcgaaaggaggaggctaaagaaaatcaacgaggccttcgaggcactgaagcggcgaactgtggccaaccccaaccagaggctgcccaaggtggagattctgcggagcgccatcagctatattgagcggctgcaggacctgctgcaccggctggatcagcaggagaagatgcaggagctgggggtggaccccttcagctacagacccaaacaagaaaatcttgagggtgcggatttcctgcgcacctgcagctcccagtggccaagtgtttccgatcattccagggggctcgtgataacggctaaggaaggaggagcaagtattgattcgtcagcctcgagtagccttcgatgcctttcttccatcgtggacagtatttcctcggaggaacgcaaactcccctgcgtggaggaagtggtggagaaggccacgaacttctctctgttaaagcaagcaggagacgtggaagaaaaccccggtcctatgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccaggcccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtag

SEQ ID NO: 41 - DNA, 3114 bp, Artificial Sequence
  Made in Lab - synthesized ASCL1-2A-MyDelta3
atggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttcgccacgaacttctctctgttaaagcaagcaggagacgtggaagaaaaccccggtcctatgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccaggcccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagaggaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtag

SEQ ID NO: 42 - DNA, 3132 bp, Artificial Sequence
  Made in Lab - synthesized MYF6-2A-MyDelta3 construct
atgatgatggacctttttgaaactggctcctatttcttctacttggatggggaaaatgttactctgcagccattagaagtggcagaaggctctcctttgtatccagggagtgatggtaccttgtccccctgccaggaccaaatgcccccggaagcggggagcgacagcagcggagaggaacatgtcctggcgcccccgggcctgcagcctccacactgccccggccagtgtctgatctgggcttgcaagacctgcaagagaaaatctgcccccactgaccggcgaaaagccgccaccctgcgcgaaaggaggaggctaaagaaaatcaacgaggccttcgaggcactgaagcggcgaactgtggccaaccccaaccagaggctgcccaaggtggagattctgcggagcgccatcagctatattgagcggctgcaggacctgctgcaccggctggatcagcaggagaagatgcaggagctgggggtggaccccttcagctacagacccaaacaagaaaatcttgagggtgcggatttcctgcgcacctgcagctcccagtggccaagtgtttccgatcattccagggggctcgtgataacggctaaggaaggaggagcaagtattgattcgtcagcctcgagtagccttcgatgcctttcttccatcgtggacagtatttcctcggaggaacgcaaactcccctgcgtggaggaagtggtggagaaggccacgaacttctctctgttaaagcaagcaggagacgtggaagaaaaccccggtcctatgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccaggcccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagaggaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtag

SEQ ID NO: 43 - protein, 287 aa, Homo sapiens
MSGSFDRKLSSILTDISSSLSCHAGSKDSPTLPESSVTDLGYYSAPQHDYYSGQPYGQTVNPYTYHHQFNLNGLAGTGAYSPKSEYTYGASYRQYGAYREQPLPAQDPVSVKEEPEAEVRMVNGKPKKVRKPRTIYSSYQLAALQRRFQKAQYLALPERAELAAQLGLTQTQVKIWFQNRRSKFKKLYKNGEVPLEHSPNNSDSMACNSPPSPALWDTSSHSTPAPARSQLPPPLPYSASPSYLDDPTNSWYHAQNLSGPHLQQQPPQPATLHHASPGPPPNPGAVY

SEQ ID NO: 44 - DNA, 864 bp, Homo sapiens
atgagtggctccttcgatcgcaagctcagcagcatcctcaccgacatctccagctcccttagctgccatgcgggctccaaggactcgcctaccctgcccgagtcttctgtcactgacctgggctactacagcgctccccagcacgattactactcgggccagccctatggccagacggtgaacccctacacctaccaccaccaattcaatctcaatgggcttgcaggcacgggcgcttactcgcccaagtcggaatatacctacggagcctcctaccggcaatacggggcgtatcgggagcagccgctgccagcccaggacccagtgtcggtgaaggaggagccggaagcagaggtgcgcatggtgaatgggaagcccaagaaggtccgaaagccgcgtacgatctactccagctaccagctggccgccctgcagcgccgcttccagaaggcccagtacctggcgctgcccgagcgcgccgagctggccgcgcagctgggcctcacgcagacacaggtgaaaatctggttccagaaccgccgttccaagttcaagaaactctacaagaacggggaggtgccgctggagcacagtcccaataacagtgattccatggcctgcaactcaccaccatcacccgccctctgggacacctcttcccactccactccggcccctgcccgcagtcagctgcccccgccgctcccatacagtgcctcccccagctacctggacgaccccaccaactcctggtatcacgcacagaacctgagtggaccccacttacagcagcagccgcctcagccagccaccctgcaccatgcctctcccgggcccccgcccaaccctggggctgtgtactga

SEQ ID NO: 45 - protein, 293 aa, Homo sapiens
MMTMTTMADGLEGQDSSKSAFMEFGQQQQQQQQQQQQQQQQQQQPPPPPPPPPQPHSQQSSPAMAGAHYPLHCLHSAAAAAAAGSHHHHHHQHHHHGSPYASGGGNSYNHRSLAAYPYMSHSQHSPYLQSYHNSSAAAQTRGDDTDQQKTTVIENGEIRFNGKGKKIRKPRTIYSSLQLQALNHRFQQTQYLALPERAELAASLGLTQTQVKIWFQNKRSKFKKLLKQGSNPHESDPLQGSAALSPRSPALPPVWDVSASAKGVSMPPNSYMPGYSHWYSSPHQDTMQRPQMM

SEQ ID NO: 46 - DNA, 882 bp, Homo sapiens
atgatgaccatgactacgatggctgacggcttggaaggccaggactcgtccaaatccgccttcatggagttcgggcagcagcagcagcagcagcagcaacagcagcagcagcagcagcagcaacagcaacagccgccgccgccgccgccgccgccgccgcagccgcactcgcagcagagctccccggccatggcaggcgcgcactaccctctgcactgcctgcactcggcggcggcggcggcagcggccggctcgcaccaccaccaccaccaccagcaccaccaccacggctcgccctacgcgtcgggcggagggaactcctacaaccaccgctcgctcgccgcctacccctacatgagccactcgcagcacagcccttacctccagtcctaccacaacagcagcgcagccgcccagacgcgaggggacgacacagatcaacaaaaaactacagtgattgaaaacggggaaatcaggttcaatggaaaagggaaaaagattcggaagcctcggaccatttattccagcctgcagctccaggctttaaaccatcgctttcagcagacacagtatctggcccttccagagagagccgaactggcagcttccttaggactgacacaaacacaggtgaagatatggtttcagaacaaacgctctaagtttaagaaactgctgaagcagggcagtaatcctcatgagagcgaccccctccagggctcggcggccctgtcgccacgctcgccagcgctgcctccagtctgggacgtttctgcctcggccaagggtgtcagtatgccccccaacagctacatgcctggctattctcactggtactcctctccacaccaggacacgatgcagagaccacagatgatgtga

SEQ ID NO: 47 - protein, 458 aa, Homo sapiens
MDSVELCLPESFSLHYEEELLCRMSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 48 - DNA, 1377 bp, Homo sapiens
atggattcggtagaactttgccttcctgaatctttttccctgcactacgaggaagagcttctctgcagaatgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcgtttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtctga

SEQ ID NO: 49 - protein, 480 aa, Homo sapiens
MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMG

SEQ ID NO: 50 - DNA, 1443 bp, Homo sapiens
atggaggtggcgcccgagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcagcccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgtcggccgccagaagagccggcacctgttgtgcaaattgtcagacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggctag

SEQ ID NO: 51 - protein, 443 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLVDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 52 - DNA, 1332 bp, Homo sapiens
atgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgtagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaa

SEQ ID NO: 53 - protein, 268 aa, Homo sapiens
MAQPLCPPLSESWMLSAAWGPTRRPPPSDKDCGRSLVSSPDSWGSTPADSPVASPARPGTLRDPRAPSVGRRGARSSRLGSGQRQSASEREKLRMRTLARALHELRRFLPPSVAPAGQSLTKIETLRLAIRYIGHLSAVLGLSEESLQRRCRQRGDAGSPRGCPLCPDDCPAQMQTRTQAEGQGQGRGLGLVSAVRAGASWGSPPACPGARAAPEPRDPPALFAEAACPEGQAMEPSPPSPLLPGDVLALLETWMPLSPLEWLPEEPK

SEQ ID NO: 54 - DNA, 807 bp, Homo sapiens
atggcccagcccctgtgcccgccgctctccgagtcctggatgctctctgcggcctggggcccaactcggcggccgccgccctccgacaaggactgcggccgctccctcgtctcgtccccagactcatggggcagcaccccagccgacagccccgtggcgagccccgcgcggccaggcaccctccgggacccccgcgccccctccgtaggtaggcgcggcgcgcgcagcagccgcctgggcagcgggcagaggcagagcgccagtgagcgggagaaactgcgcatgcgcacgctggcccgcgccctgcacgagctgcgccgctttctaccgccgtccgtggcgcccgcgggccagagcctgaccaagatcgagacgctgcgcctggctatccgctatatcggccacctgtcggccgtgctaggcctcagcgaggagagtctccagcgccggtgccggcagcgcggtgacgcggggtcccctcggggctgcccgctgtgccccgacgactgccccgcgcagatgcagacacggacgcaggctgaggggcaggggcaggggcgcgggctgggcctggtatccgccgtccgcgccggggcgtcctggggatccccgcctgcctgccccggagcccgagctgcacccgagccgcgcgacccgcctgcgctgttcgccgaggcggcgtgccctgaagggcaggcgatggagccaagcccaccgtccccgctccttccgggcgacgtgctggctctgttggagacctggatgcccctctcgcctctggagtggctgcctgaggagcccaagtga

SEQ ID NO: 55 - protein, 242 aa, Homo sapiens
MMMDLFETGSYFFYLDGENVTLQPLEVAEGSPLYPGSDGTLSPCQDQMPPEAGSDSSGEEHVLAPPGLQPPHCPGQCLIWACKTCKRKSAPTDRRKAATLRERRRLKKINEAFEALKRRTVANPNQRLPKVEILRSAISYIERLQDLLHRLDQQEKMQELGVDPFSYRPKQENLEGADFLRTCSSQWPSVSDHSRGLVITAKEGGASIDSSASSSLRCLSSIVDSISSEERKLPCVEEVVEK

SEQ ID NO: 56 - DNA, 729 bp, Homo sapiens
atgatgatggacctttttgaaactggctcctatttcttctacttggatggggaaaatgttactctgcagccattagaagtggcagaaggctctcctttgtatccagggagtgatggtaccttgtccccctgccaggaccaaatgcccccggaagcggggagcgacagcagcggagaggaacatgtcctggcgcccccgggcctgcagcctccacactgccccggccagtgtctgatctgggcttgcaagacctgcaagagaaaatctgcccccactgaccggcgaaaagccgccaccctgcgcgaaaggaggaggctaaagaaaatcaacgaggccttcgaggcactgaagcggcgaactgtggccaaccccaaccagaggctgcccaaggtggagattctgcggagcgccatcagctatattgagcggctgcaggacctgctgcaccggctggatcagcaggagaagatgcaggagctgggggtggaccccttcagctacagacccaaacaagaaaatcttgagggtgcggatttcctgcgcacctgcagctcccagtggccaagtgtttccgatcattccagggggctcgtgataacggctaaggaaggaggagcaagtattgattcgtcagcctcgagtagccttcgatgcctttcttccatcgtggacagtatttcctcggaggaacgcaaactcccctgcgtggaggaagtggtggagaagtaa

SEQ ID NO: 57 - protein, 1241 aa, Artificial Sequence
  Made in Lab - synthesized MYOCD-2A-ASCL1 construct
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQWATNFSLLKQAGDVEENPGPMESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 58 - protein, 1247 aa, Artificial Sequence
  Made in Lab - synthesized MYOCD-2A-MYF6 construct
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQWATNFSLLKQAGDVEENPGPMMMDLFETGSYFFYLDGENVTLQPLEVAEGSPLYPGSDGTLSPCQDQMPPEAGSDSSGEEHVLAPPGLQPPHCPGQCLIWACKTCKRKSAPTDRRKAATLRERRRLKKINEAFEALKRRTVANPNQRLPKVEILRSAISYIERLQDLLHRLDQQEKMQELGVDPFSYRPKQENLEGADFLRTCSSQWPSVSDHSRGLVITAKEGGASIDSSASSSLRCLSSIVDSISSEERKLPCVEEVVEK

SEQ ID NO: 59 - protein, 1037 aa, Artificial Sequence
  Made in Lab - synthesized MyDelta3-2A-ASCL1 construct
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQWATNFSLLKQAGDVEENPGPMESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 60 - protein, 1043 aa, Artificial Sequence
  Made in Lab - synthesized MyDeltal3-2A-MYF6 construct
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQWATNFSLLKQAGDVEENPGPMMMDLFETGSYFFYLDGENVTLQPLEVAEGSPLYPGSDGTLSPCQDQMPPEAGSDSSGEEHVLAPPGLQPPHCPGQCLIWACKTCKRKSAPTDRRKAATLRERRRLKKINEAFEALKRRTVANPNQRLPKVEILRSAISYIERLQDLLHRLDQQEKMQELGVDPFSYRPKQENLEGADFLRTCSSQWPSVSDHSRGLVITAKEGGASIDSSASSSLRCLSSIVDSISSEERKLPCVEEVVEK

SEQ ID NO: 61 - protein, 1241 aa, Artificial Sequence
  Made in Lab - synthesized ASCL1-2A-MYOCD construct
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWFATNFSLLKQAGDVEENPGPMTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 62 - protein, 1247 aa, Artificial Sequence
  Made in Lab - synthesized MYF6-2A-MYOCD construct
MMMDLFETGSYFFYLDGENVTLQPLEVAEGSPLYPGSDGTLSPCQDQMPPEAGSDSSGEEHVLAPPGLQPPHCPGQCLIWACKTCKRKSAPTDRRKAATLRERRRLKKINEAFEALKRRTVANPNQRLPKVEILRSAISYIERLQDLLHRLDQQEKMQELGVDPFSYRPKQENLEGADFLRTCSSQWPSVSDHSRGLVITAKEGGASIDSSASSSLRCLSSIVDSISSEERKLPCVEEVVEKATNFSLLKQAGDVEENPGPMTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 63 - protein, 1037 aa, Artificial Sequence
  Made in Lab - synthesized ASCL1-2A-MyDelta3 construct
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWFATNFSLLKQAGDVEENPGPMTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 64 - protein, 1043 aa, Artificial Sequence
  Made in Lab - synthesized MYF6-2A-MyDetal3
MMMDLFETGSYFFYLDGENVTLQPLEVAEGSPLYPGSDGTLSPCQDQMPPEAGSDSSGEEHVLAPPGLQPPHCPGQCLIWACKTCKRKSAPTDRRKAATLRERRRLKKINEAFEALKRRTVANPNQRLPKVEILRSAISYIERLQDLLHRLDQQEKMQELGVDPFSYRPKQENLEGADFLRTCSSQWPSVSDHSRGLVITAKEGGASIDSSASSSLRCLSSIVDSISSEERKLPCVEEVVEKATNFSLLKQAGDVEENPGPMTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 65 - DNA, 88 bp, Homo sapiens
acaatgctttgctagagctggtaaaatggaaccaaatcgcctcttcaatggatttggtccccttcaaccagctgtagctatgcattga

SEQ ID NO: 66 - DNA, 102 bp, Homo sapiens
gggagccaaatgctttgctagagctggtaaaatggaaccaaatcgactgtccaatggatttggtccccttcaaccagctgtagctgtgcattgatggcgccg

SEQ ID NO: 67 - DNA, 119 bp, Homo sapiens
cctcagaagaaagatgccccctgctctggctggtcaaacggaaccaagtccgtcttcctgagaggtttggtccccttcaaccagctacagcagggctggcaatgcccagtccttggaga

SEQ ID NO: 68 - DNA, 87 bp, Homo sapiens
cactgttctatggttagttttgcaggtttgcatccagctgtgtgatattctgctgtgcaaatccatgcaaaactgactgtggtagtg

SEQ ID NO: 69 - DNA, 96 bp, Homo sapiens
acattgctacttacaattagttttgcaggtttgcatttcagcgtatatatgtatatgtggctgtgcaaatccatgcaaaactgattgtgataatgt

SEQ ID NO: 70 - DNA, 95 bp, Homo sapiens
ctcatctgtctgttgggctggaggcagggcctttgtgaaggcgggtggtgctcagatcgcctctgggcccttcctccagccccgaggcggattca

SEQ ID NO: 71 - DNA, 71 bp, Homo sapiens
tgggaaacatacttctttatatgcccatatggacctgctaagctatggaatgtaaagaagtatgtatctca

SEQ ID NO: 72 - DNA, 85 bp, Homo sapiens
acctactcagagtacatacttctttatgtacccatatgaacatacaatgctatggaatgtaaagaagtatgtatttttggtaggc

SEQ ID NO: 73 - DNA, 112 bp, Homo sapiens
agacgaggagttaagagttcattcggctgtccagatgtatccaagtaccctgtgttatttggcaataaatacatctgggcaactgactgaacttttcacttttcatgactca

SEQ ID NO: 74 - DNA, 75 bp, Homo sapiens
tcatccctgggtggggatttgttgcattacttgtgttctatataaagtattgcacttgtcccggcctgtggaaga

SEQ ID NO: 75 - DNA, 71 bp, Homo sapiens
gtagcactaaagtgcttatagtgcaggtagtgtttagttatctactgcattatgagcacttaaagtactgc

SEQ ID NO: 76 - DNA, 69 bp, Homo sapiens
agtaccaaagtgctcatagtgcaggtagttttggcatgactctactgtagtatgggcacttccagtact

SEQ ID NO: 77 - DNA, 95 bp, Homo sapiens
cggccggccctgggtccatcttccagtacagtgttggatggtctaattgtgaagctcctaacactgtctggtaaagatggctcccgggtgggttc

SEQ ID NO: 78 - DNA, 65 bp, Homo sapiens
ctgttaatgctaatcgtgataggggtttttgcctccaactgactcctacatattagcattaacag

SEQ ID NO: 79 - DNA, 84 bp, Homo sapiens
gtcagaataatgtcaaagtgcttacagtgcaggtagtgatatgtgcatctactgcagtgaaggcacttgtagcattatggtgac

SEQ ID NO: 80 - DNA, 84 bp, Homo sapiens
gcatccgggttgaggtagtaggttgtatggtttagagttacaccctgggagttaactgtacaaccttctagctttccttggagc

SEQ ID NO: 81 - DNA, 110 bp, Homo sapiens
cgcctcagagccgcccgccgttcctttttcctatgcatatacttctttgaggatctggcctaaagaggtatagggcatgggaaaacggggcggtcgggtcctccccagcg

SEQ ID NO: 82 - DNA, 90 bp, Homo sapiens
ccgggcccctgtgagcatcttaccggacagtgctggatttcccagcttgactctaacactgtctggtaacgatgttcaaaggtgacccgc

SEQ ID NO: 83 - DNA, 86 bp, Homo sapiens
tgcttcccgaggccacatgcttctttatatccccatatggattactttgctatggaatgtaaggaagtgtgtggtttcggcaagtg

SEQ ID NO: 84 - DNA, 94 bp, Homo sapiens
catgctgtgtgtggtaccctactgcagacagtggcaatcatgtataattaaaaatgattggtacgtctgtgggtagagtactgcatgacacatg

SEQ ID NO: 85 - DNA, 91 bp, Homo sapiens
catgctgtgtgtggtaccctactgcagacagtggcaatcatgtataattaaaaatgattggtacgtctgtgggtagagtactgcatgacac

SEQ ID NO: 86 - DNA, 109 bp, Homo sapiens
atcaagattagaggctctgctctccgtgttcacagcggaccttgatttaatgtcatacaattaaggcacgcggtgaatgccaagagcggagcctacggctgcacttgaa

SEQ ID NO: 87 - DNA, 87 bp, Homo sapiens
tgagggcccctctgcgtgttcacagcggaccttgatttaatgtctatacaattaaggcacgcggtgaatgccaagagaggcgcctcc

SEQ ID NO: 88 - DNA, 66 bp, Homo sapiens
agggctcctgactccaggtcctgtgtgttacctagaaatagcactggacttggagtcagaaggcct

SEQ ID NO: 89 - DNA, 79 bp, Homo sapiens
ctgactccagtgtccaggccaggggcagacagtggacagagaacagtgcccaagaccactggacttggagtcaggacat

SEQ ID NO: 90 - DNA, 83 bp, Homo sapiens
acaggaacactggacttggtgtcagatgggatgagccctggctctgtttcctagcagcaatctgatcttgagctagtcactgg

SEQ ID NO: 91 - DNA, 76 bp, Homo sapiens
gggagcactggactaggagtcagaaggtggagttctgggtgctgttttcccactcttgggccctgggcatgttctg

SEQ ID NO: 92 - DNA, 102 bp, Homo sapiens
ggtcctctgactctcttcggtgacgggtattcttgggtggataatacggattacgttgttattgcttaagaatacgcgtagtcgaggagagtaccagcggca

SEQ ID NO: 93 - DNA, 118 bp, Homo sapiens
gcaggtgaactggcaggccaggaagaggaggaagccctggaggggctggaggtgatggatgttttcctccggttctcagggctccacctctttcgggccgtagagccagggctggtgc

SEQ ID NO: 94 - DNA, 68 bp, Homo sapiens
ctccggtgcctactgagctgatatcagttctcattttacacactggctcagttcagcaggaacaggag

SEQ ID NO: 95 - DNA, 110 bp, Homo sapiens
gagctgcttgcctccccccgtttttggcaatggtagaactcacactggtgaggtaacaggatccggtggttctagacttgccaactatggggcgaggactcagccggcac

SEQ ID NO: 96 - DNA, 68 bp, Homo sapiens
cctctactttaacatggaggcacttgctgtgacatgacaaaaataagtgcttccatgtttgagtgtgg

SEQ ID NO: 97 - DNA, 78 bp, Homo sapiens
tggccgattttggcactagcacatttttgcttgtgtctctccgctctgagcaatcatgtgcagtgccaatatgggaaa

SEQ ID NO: 98 - DNA, 72 bp, Homo sapiens
agatactgtaaacatcctacactctcagctgtggaaagtaagaaagctgggagaaggctgtttactctttct

SEQ ID NO: 99 - DNA, 73 bp, Homo sapiens
cctggcactgagaactgaattccataggctgtgagctctagcaatgccctgtggactcagttctggtgcccgg

SEQ ID NO: 100 - DNA, 22 bp, Homo sapiens
tttggtccccttcaaccagctg

SEQ ID NO: 101 - DNA, 22 bp, Homo sapiens
tttggtccccttcaaccagctg

SEQ ID NO: 102 - DNA, 22 bp, Homo sapiens
tttggtccccttcaaccagcta

SEQ ID NO: 103 - DNA, 23 bp, Homo sapiens
tgtgcaaatccatgcaaaactga

SEQ ID NO: 104 - DNA, 23 bp, Homo sapiens
tgtgcaaatccatgcaaaactga

SEQ ID NO: 105 - DNA, 20 bp, Homo sapiens
cctctgggcccttcctccag

SEQ ID NO: 106 - DNA, 22 bp, Homo sapiens
tggaatgtaaagaagtatgtat

SEQ ID NO: 107 - DNA, 22 bp, Homo sapiens
tggaatgtaaagaagtatgtat

SEQ ID NO: 108 - DNA, 22 bp, Homo sapiens
ttcattcggctgtccagatgta

SEQ ID NO: 109 - DNA, 22 bp, Homo sapiens
tattgcacttgtcccggcctgt

SEQ ID NO: 110 - DNA, 23 bp, Homo sapiens
taaagtgcttatagtgcaggtag

SEQ ID NO: 111 - DNA, 23 bp, Homo sapiens
caaagtgctcatagtgcaggtag

SEQ ID NO: 112 - DNA, 22 bp, Homo sapiens
taacactgtctggtaaagatgg

SEQ ID NO: 113 - DNA, 24 bp, Homo sapiens
ttaatgctaatcgtgataggggtt

SEQ ID NO: 114 - DNA, 23 bp, Homo sapiens
caaagtgcttacagtgcaggtag

SEQ ID NO: 115 - DNA, 22 bp, Homo sapiens
tgaggtagtaggttgtatggtt

SEQ ID NO: 116 - DNA, 22 bp, Homo sapiens
ttcctatgcatatacttctttg

SEQ ID NO: 117 - DNA, 22 bp, Homo sapiens
taacactgtctggtaacgatgt

SEQ ID NO: 118 - DNA, 22 bp, Homo sapiens
tggaatgtaaggaagtgtgtgg

SEQ ID NO: 119 - DNA, 22 bp, Homo sapiens
tgattggtacgtctgtgggtag

SEQ ID NO: 120 - DNA, 22 bp, Homo sapiens
tgattggtacgtctgtgggtag

SEQ ID NO: 121 - DNA, 22 bp, Homo sapiens
taaggcacgcggtgaatgccaa

SEQ ID NO: 122 - DNA, 22 bp, Homo sapiens
taaggcacgcggtgaatgccaa

SEQ ID NO: 123 - DNA, 22 bp, Homo sapiens
actggacttggagtcagaaggc

SEQ ID NO: 124 - DNA, 19 bp, Homo sapiens
actggacttggagtcagga

SEQ ID NO: 125 - DNA, 21 bp, Homo sapiens
actggacttggtgtcagatgg

SEQ ID NO: 126 - DNA, 21 bp, Homo sapiens
actggactaggagtcagaagg

SEQ ID NO: 127 - DNA, 23 bp, Homo sapiens
ttattgcttaagaatacgcgtag

SEQ ID NO: 128 - DNA, 23 bp, Homo sapiens
aggaagccctggaggggctggag

SEQ ID NO: 129 - DNA, 22 bp, Homo sapiens
tggctcagttcagcaggaacag

SEQ ID NO: 130 - DNA, 24 bp, Homo sapiens
tttggcaatggtagaactcacact

SEQ ID NO: 131 - DNA, 23 bp, Homo sapiens
taagtgcttccatgtttgagtgt

SEQ ID NO: 132 - DNA, 23 bp, Homo sapiens
tttggcactagcacatttttgct

SEQ ID NO: 133 - DNA, 23 bp, Homo sapiens
tgtaaacatcctacactctcagc

SEQ ID NO: 134 - DNA, 23 bp, Homo sapiens
tgagaactgaattccataggctg

SEQ ID NO: 135 - DNA, 22 bp, Artificial Sequence
  miR-208 binding site
acaaaccttttgttcgtcttat

SEQ ID NO: 136 - DNA, 22 bp, Artificial Sequence
  miR-208 binding site miR-208_4
aaaatatatgtaatcgtcttaa

SEQ ID NO: 137 - DNA, 22 bp, Artificial Sequence
  miR-208 binding site
tgaaaccttttgttcgtcttat

SEQ ID NO: 138 - DNA, 97 bp, Artificial Sequence
  3 prime UTR
acaaaccttttgttcgtcttataaaacaaaccttttgttcgtcttataaaacaaaccttttgttcgtcttataaaacaaaccttttgttcgtcttat

SEQ ID NO: 139 - DNA, 47 bp, Artificial Sequence
  3 prime UTR
acaaaccttttgttcgtcttataaaacaaaccttttgttcgtcttat

SEQ ID NO: 140 - DNA, 94 bp, Artificial Sequence
  3 prime UTR
aaaatatatgtaatcgtcttaaaaaaaatatatgtaatcgtcttaaaaaaaatatatgtaatcgtcttaaaaaaaatatatgtaatcgtcttaa

SEQ ID NO: 141 - RNA, 22 nt, Artificial Sequence
  miR-1 binding site miR1-1/1-2
uauguaugaagaaauguaaggu

SEQ ID NO: 142 - RNA, 22 nt, Artificial Sequence
  miR-1 binding site miR1_4
auacauacuucuuuacauucca

SEQ ID NO: 143 - RNA, 22 nt, Artificial Sequence
  miR-1 binding site miR1_4mut
auacauacuucuuuacagagca

SEQ ID NO: 144 - RNA, 21 nt, Artificial Sequence
  miR-1 binding site MymiR1_4
acgaugucauuuaacauuccg

SEQ ID NO: 145 - RNA, 21 nt, Artificial Sequence
  miR-1 binding site MymiR1_4mut
acgaugucauuuaacagagcg

SEQ ID NO: 146 - RNA, 22 nt, Artificial Sequence
  miR-1 binding site miR1_1mis2
auacauaguuguuuacauucca

SEQ ID NO: 147 - RNA, 22 nt, Artificial Sequence
  miR-1 binding site miR1_1mis3
auacauaguugucuacauucca

SEQ ID NO: 148 - RNA, 22 nt, Artificial Sequence
  miR-208 binding site miR-208b
uguuuggaaaacaagcagaaua

SEQ ID NO: 149 - RNA, 22 nt, Artificial Sequence
  miR-208 binding site miR208
acaaaccuuuuguucgucuuau

SEQ ID NO: 150 - RNA, 22 nt, Artificial Sequence
  miR-208 binding site miR208_melt
ugaaaccuuuuguucgucuuau

SEQ ID NO: 151 - RNA, 22 nt, Artificial Sequence
  miR-208 binding site miR208_meltmut
ugaaaccuuuuguucgugagau

SEQ ID NO: 152 - DNA, 22 bp, Artificial Sequence
  microRNA binding site microRNA binding site miR1_4
atacatacttctttacattcca

SEQ ID NO: 153 - DNA, 22 bp, Artificial Sequence
  microRNA binding site miR1_4mut
atacatacttctttacagagca

SEQ ID NO: 154 - DNA, 21 bp, Artificial Sequence
  microRNA binding site MymiR1_4
acgatgtcatttaacattccg

SEQ ID NO: 155 - DNA, 21 bp, Artificial Sequence
  microRNA binding site MymiR1_4mut
acgatgtcatttaacagagcg

SEQ ID NO: 156 - DNA, 22 bp, Artificial Sequence
  microRNA binding site miR1_1
atacatacttctttacattcca

SEQ ID NO: 157 - DNA, 22 bp, Artificial Sequence
  microRNA binding site miR1_2
atacatacttctttacattcca

SEQ ID NO: 158 - DNA, 22 bp, Artificial Sequence
  microRNA binding site miR1_3
atacatacttctttacattcca

SEQ ID NO: 159 - DNA, 22 bp, Artificial Sequence
  microRNA binding site miR1_1mis2
atacatagttgtttacattcca

SEQ ID NO: 160 - DNA, 22 bp, Artificial Sequence
  microRNA binding site miR1_1mis3
atacatagttgtctacattcca

SEQ ID NO: 161 - DNA, 20 bp, Artificial Sequence
  microRNA binding site miR1_1TB4
aatatgcactgtacattcca

SEQ ID NO: 162 - DNA, 22 bp, Artificial Sequence
  microRNA binding site 208_4mut
acaaaccttttgttcgtcgaat

SEQ ID NO: 163 - DNA, 22 bp, Artificial Sequence
  microRNA binding site 208_4mut3
tgaaaccttttgttcgtgagat

SEQ ID NO: 164 - DNA, 22 bp, Artificial Sequence
  microRNA binding site 208_moMEL1
tgaaagcttttgttcgtcttat

SEQ ID NO: 165 - DNA, 22 bp, Artificial Sequence
  microRNA binding site 208_moMEL2
tgatagcttttgttcgtcttat

SEQ ID NO: 166 - DNA, 22 bp, Artificial Sequence
  microRNA binding site 208_chim
atacatacttcgttcgtcttat